(19) United States
(12) Patent Application Publication
(10) Pub. No.: US 2026/0015402 A1
(43) Pub. Date: Jan. 15, 2026

(54) SMALL MOLECULE ADAPTER REGULATED, TARGET SPECIFIC CHIMERIC ANTIGEN RECEPTOR BEARING T CELLS (SMART CARS)

(71) Applicant: YALE UNIVERSITY, NEW HAVEN, CT (US)

(72) Inventors: David Spiegel, Hamden, CT (US); Viswanathan Muthusamy, New Haven, CT (US); Jesse Collinski, San Jose, CA (US)

(21) Appl. No.: 19/247,240

(22) Filed: Jun. 24, 2025

Related U.S. Application Data
(63) Continuation of application No. 16/633,101, now abandoned.
(60) Provisional application No. 62/528,314, filed on Jul. 3, 2017.

(51) Int. Cl. C07K 14/725; A61K 40/11; A61K 40/31; A61K 40/42; A61K 47/54; A61K 47/55; C07K 14/705; C07K 14/715; C07K 16/30; C12N 5/0783

(52) U.S. Cl. CPC C07K 14/7051; A61K 40/11; A61K 40/31; A61K 40/4276; A61K 47/545; A61K 47/55; C07K 14/70521; C07K 14/70578; C07K 14/7151; C07K 16/3069; C12N 5/0636; C12N 5/0638; A61K 2239/24; C07K 2317/31

(57) ABSTRACT

In one embodiment, the invention provides a chimeric antigen receptor (CAR) T cell which is conjugated to a bi-functional molecule which is specific for both an extracellular binding domain of the chimeric antigen receptor (CAR) T cell and prostate-specific membrane antigen (PSMA). The chimeric antigen receptor (CAR) T cell contains a T cell signaling domain and the extracellular binding domain of the chimeric antigen receptor (CAR) T cell is not specific for prostate-specific membrane antigen (PSMA). Compositions and methods of treatment using these CAR T cells are also disclosed.

### RELATED APPLICATIONS

This application claims the benefit of priority of United States provisional application serial number U.S. 62/528,314, filed Jul. 3, 2017, the entire contents of said application being incorporated by reference herein.

### FIELD OF THE INVENTION

The invention provides small molecule adapter regulated, target specific chimeric antigen receptor bearing T-Cells (SMART CARs) and related anticancer methods of treatment, pharmaceutical compositions, diagnostic assays and kits. Chimeric antigen receptor (CAR) T cell-bi-functional molecule conjugates as described and claimed herein are particularly useful in the treatment of prostate cancer, including metastatic and recurrent prostate cancer.

### BACKGROUND OF THE INVENTION

It has been predicted that one out of every six American men will develop prostate cancer in their lifetime. See American Cancer Society, Cancer Facts and Figures 2008. Atlanta: American Cancer Society; 2008. Despite recent advances in both prostate cancer detection and treatment, it remains one of the leading causes of cancer-related death among the American male population.

When prostate cancer is diagnosed prior to metastasis, the patient has a greater then 99% chance of survival. The most successful means for treating prostate cancer at this stage is a radical prostatectomy. Unfortunately, this surgery carries with it the risk of severing nerves and blood vessels associated with sexual organs and the bladder, and can potentially result in impotency or incontinency. Radiation therapy is yet another commonly used procedure that carries the risk of impotency. Half the patients who undergo radiation therapy for prostate cancer become impotent within 2 years of treatment. In addition to the adverse affects associated with these procedures, they are significantly less effective in patients whose cancer has already delocalized or metastasized on diagnosis. In these cases, patients generally undergo even more invasive procedures such as hormonal therapy or chemotherapy. Unfortunately, most patients eventually stop responding to hormonal therapy and the most successful chemotherapeutic, Taxotere, only prolongs the life of advanced prostate cancer patients by 2.5 months on average.

As explained in Sadelain, et al., “The Basic Principles of Chimeric Antigen Receptor Design”, Cancer Discovery, April 2013, 3; 388, “[c]himeric antigen receptors (CAR) are recombinant receptors for antigen, which, in a single molecule, redirect the specificity and function of T lymphocytes and other immune cells. The general premise for their use in cancer immunotherapy is to rapidly generate tumor-targeted T cells, bypassing the barriers and incremental kinetics of active immunization.” CAR T cell therapies have been used in the treatment of prostate cancer. Sanchez, et al., “Combining T-cell immunotherapy and anti-androgen therapy for prostate cancer”, Prostate Cancer Prostatic Dis. 2013 June; 16(2):123-31. CAR T cells have been successfully developed and approved for use in treatment of B-cell leukemia. Advanced phase clinical trials are currently underway to explore the possibility and efficacy of their usage in targeting other tumor types including solid tumors. CAR T cell therapies are advantageous because they are not MHC restricted and, as a single simple protein, effect antigen binding and signaling functions offered by the more complex T cell receptors.

There are significant safety concerns associated with current CAR T cell-based therapies; these concerns include the targeted destruction of normal tissues, cytokine storms associated with large-scale immune responses, and the toxicity of the different conditioning regimens used in conjunction with adoptive T-cell therapies. Sadelain, et al. Cancer Discovery, April 2013 3; 388. Given the clinical potential of CAR T cell therapies, the disadvantages of known treatments of non-metastatic prostate cancer and the poor long-term prognosis associated with a diagnosis of metastatic prostate cancer, there is a profound clinical need for chimeric antigen receptor (CAR) T cells which target prostate cancer cells effectively and which evidence an improved safety profile when compared to known CAR T cells.

### SUMMARY OF THE INVENTION

The present invention is directed to specific small molecule intermediates which are used to bridge a target diseased cell and an effector T cell and the inventors have engineered CAR T cell-small molecule conjugates which exhibit significant clinical potential as safe and effective anti-cancer agents.

In one embodiment, the present invention provides a chimeric antigen receptor (CAR) T cell which is conjugated to a bi-functional molecule. The chimeric antigen receptor (CAR) of the CAR T cell comprises an antigen binding domain, a hinge domain, a transmembrane domain (preferably, a human CD28 transmembrane domain), a co-stimulatory signaling region, an optional secondary co-stimulatory signaling region (eg, ICOS/inducible costimulatory region such as 4-1BB) and a signaling domain (often, a CD3 zeta (CD3-ζ) signaling domain) and the bi-functional molecule comprises a chimeric antigen receptor binding moiety (CARBM) which binds to said CAR at the antigen binding domain and a cancer binding moiety (CBM), wherein the cancer binding moiety is conjugated to the CARBM through a linker which optionally and preferably includes at least one connector group (CON) as otherwise described herein. It is noted that the CAR antigen binding domain is not a prostate-specific membrane antigen (PSMA) domain, but is a domain which can bind or conjugate to one end of the bifunctional molecule, often irreversibly (e.g. by forming a covalent bond).

In embodiments, the present invention is directed to engineered cells (T cells) which express a chimeric antigen receptor (CAR) as otherwise described herein. In certain embodiments the present invention is directed to engineered cells (T cells) which express a chimeric antigen receptor as described herein which is bound to a bifunctional molecule which comprises a moiety which binds to the antigen binding domain of said chimeric antigen receptor. In alternative embodiments, the bifunctional molecule comprises a moiety which is acted on by the antigen binding domain (when the antigen binding domain is a halotag, snaptap or cliptag protein) to produce a covalent bond which attaches the bifunctional molecule to the antigen binding domain of the chimeric antigen receptor.

In embodiments, the CAR antigen binding domain is preferably a member of the FKBP family as described herein, a haloalkane dehalogenase/halotag protein (available from Promega Corporation), a snap-tag protein (a human O6-alkylguanine-DNA alkyltransferase (hAGT) variant which accepts O6-benzyl guanine derivatives, see Juillerat, et al., Chemistry and Biology, 10 (4): 313-317, April, 2003) available from New England Biolabs, Inc. or a clip-tag protein (which has been further engineered from the snaptag protein to accept O2-benzyl cytosine derivatives), see Gautier, et al., Chemistry and Biology, 15 (2): 128-136, February 2008), available from New England Biololabs, Inc.

The bi-functional molecule is specific for the antigen binding domain of the chimeric antigen receptor (CAR) T cell at one end of the molecule and a prostate-specific membrane antigen (PSMA) at the other end of the molecule linked together by a linker group which optionally comprises a connector (CON) group. In certain preferred aspects, the bifunctional molecule comprises at one end a moiety which is a substrate of a halotag protein, a snap-tag protein or clip-tag protein which is acted on by the protein and is conjugated from the bifunctional molecule to the CAR antigen binding domain through a covalent bond between the bifunctional molecule and the antigen binding domain. In this manner, the bifunctional molecule can become covalently “anchored” to the CAR T cell, but disposed extracellularly to function as a targeting moiety for the CAR T cell and a cancer cell. Often, the CAR antigen binding domain is a halotag protein (haloalkane dehalogenase), a snaptag protein or a cliptag protein. Through the use of a bi-functional molecule, the CARBM may be modified to bind to any number of antigen binding domains and the cancer binding moiety (CBM or PBM) may be modified to accommodate a large number of moieties which can be used to target specific cancer cell types. In preferred embodiments, the CBM/PBM is a prostate specific membrane antigen (PSMA) and the target cell is any cancer cell which PSMA on its surface at high levels, often cells which overexpress or hyperexpress PSMA. Often the cancer cell is a prostate cancer cell or a metastatic prostate cancer cell.

In one embodiment, the antigen binding domain of the chimeric antigen receptor (CAR) T cell is HaloTag® protein (a 34 kDa, monomeric derivative of dehalogenase) (Promega Biosciences San Luis Obispo, CA) and the cognate bi-functional molecule comprises at one end a C₃-C₁₀ haloalkane which binds to the HaloTag protein/dehalogenase and is acted upon by the dehalogenase, forming a covalent bond with the (CAR) T cell through the antigen binding domain.

In one embodiment, the antigen binding domain of the chimeric antigen receptor (CAR) T cell is a SnapTag (a human O6-alkylguanine-DNA alkyltransferase (hAGT) variant which accepts O6-benzyl guanine derivatives), available from New England Biolabs, Inc. and the cognate bifunctional molecule comprises at one end a O6-benzyl guanine group which binds to the Snaptag protein/alkyltransferase and is acted upon by the SnapTag protein/alkyltransferase, thus forming a covalent bond with the benzyl group of the bifunctional molecule (through a sulfur linkage on the protein) and the (CAR) T cell.

In one embodiment, the antigen binding domain of the chimeric antigen receptor (CAR) T cell is a ClipTag protein (e.g., hAGT variant engineered to accept O2-benzyl cytosine derivatives), available from New England Biololabs, Inc. and the cognate bifunctional molecule comprises at one end a O2-benzyl cytosine group which binds to the ClipTag protein and is acted upon by the ClipTag protein, thus forming a covalent bond with the benzyl group of the bifunctional molecule (most often, through a sulfur linkage on the protein) and the (CAR) T cell.

Alternatively, the antigen binding domain of the chimeric antigen receptor (CAR) T cell is a member of the immunophilin (FKBP) family of proteins (FK506 binding proteins), preferably a human protein and is preferably selected from the group consisting of FKBP3 (UniProtKB/Swiss-Prot Accession Number Q00688.1, same as FKBP25), FKPB5 (Q13451.2), FKBP9 (O95302.2), FKBP12 (P62942.2), FKBP12.6 (P68106.2), FKBP13 (P26885.2), FKBP15 (Q5T1M5.2), FKBP22 (Q9NWM8), FKBP36 (O75344.1), FKBP38 (Q14318.2), FKBP51 (Q02790.3), FKBP65 (Q9FJL3.1) and FKBP133 (Q6P9Q6.2) or an isoform or fragment thereof which binds to a FKBP binding moiety and the bi-functional molecule contains a moiety which binds to the FKBP (FKBP binding moiety) and which is selected from the group consisting of FK506 (tacrolimus), a FK506 derivative or a rapalog.

The antigen binding domain of the chimeric antigen receptor (CAR) T cell can be an amino acid sequence that exhibits substantial homology with or substantial similarity to a FKBP as described above and at a minimum can comprise a FKBP binding site.

Useful FK506 derivatives which may be included in bi-functional molecules according to the present invention include but are not limited to moieties of tacrolimus (FK506), FK1706, meridamycin, normeridamycin, ILS920, Way-124466, Wye-592, L685-818, VX-10,367, VX-710 (Biricodar), VX-853 (Timcodar), JNJ460/GM284, GP11046, GP11485 and DM-CHX; useful rapologs include but are not limited to rapamycin (sirolimus), temsirolimus (CCI 779), everolimus (RAD001) and ridaforolimus/deforolimus (AP-23573). Specific FK506 derivative chemical moieties useful in the present invention include the moieties which are presented in FIG. 25 hereof.

Useful T cell signaling domains include human CD8-alpha protein, human CD28 protein, human CD3-zeta protein (CD3-ζ or TCR-ζ), human F_{c}Rγ protein, CD27 protein, OX40 protein, human 4-1BB protein, variants of any of the forgoing and fusion proteins comprising two or more of the foregoing. A preferred signaling domain comprises human CD3-zeta protein.

In embodiments, the co-stimulatory signaling domain includes CD28, CD2, 4-1BB (CD137) and OX-40 (CD124). In certain embodiments, the co-stimulatory signaling domain comprises two co-stimulatory domains, for example, human CD28 protein and human 4-1BB protein in order to promote T cell quantity and strength of activation, potency, phenotype of T-cell and cytokine upregulation See, for example, Zhong, et al., “Chimeric antigen receptors combining 4-1BB and CD28 signaling domains augment PI3kinase/AKT/Bcl-XL activation and CD8+ T cell-mediated tumor eradication”, Mol. Ther., 2010 February; 18(2):413-20.

T cells which are used in the present invention include but are not limited to a helper (CD4⁺) T cell, cytotoxic (CD8⁺) T cell, central memory T cell (T_{CM} cell), an effector memory T cells (T_{EM} cell or T_{EMRA} cell), a regulatory (suppressor or T_{reg}) T cell or a natural killer T cell (NKT cell). These T cells are modified to express a CAR polypeptide as otherwise described herein to which is conjugated a bi-functional molecule which also contains a cancer binding moiety which increases the ability of the T cell to target cancer cells and enhance anti-cancer therapy.

In a preferred embodiment, the antigen binding domain of the chimeric antigen receptor (CAR) T cell is FKBP12 and the bi-functional molecule contains a FK506 (tacrolimus) moiety as described herein which binds to FKBP12.

In preferred embodiments of the present invention, the portion of the bi-functional molecule which binds to PSMA is a glutamate urea derivative (the moiety “B” in the structures below, also referred to as a cancer binding moiety “CBM” or prostate binding moiety “PBM”). In preferred embodiments, the CBM or PBM is linked to the CARBM through a linker group which optionally and preferably contains a CON group, which is preferably a triazole group.

In another embodiment, the present invention provides an engineered polypeptide including a chimeric antigen receptor (CAR) to which is covalently attached a bifunctional molecule.

In certain preferred embodiments, the bi-functional molecule has the formula:

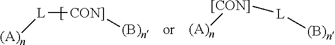

wherein:
n is 1-3, preferably 1 or 2, most often 1;
n′ is 1-6, preferably 1 or 2, most often 1
(a) A is a moiety (“a CAR binding moiety” or “CARBM”) which binds to the antigen binding domain of the chimeric antigen receptor (CAR) T cell and is (1) a C₃-C₁₀ haloalkane (preferably, a C₃-C₈ chloroalkane, more preferably chlorohexane) if the antigen binding domain comprises a halotag protein, (2) a O6 benzyl guanine moiety if the antigen binding moiety comprises a snaptag protein, (3) a O2 benzyl cytosine moiety if the antigen binding moiety comprises a cliptag protein or (4) a FK506 (tacrolimus), a FK506 derivative or a rapalog if the antigen binding domain is a FKBP or an amino acid sequence that exhibits substantial homology with or substantial similarity to a FKBP and that at a minimum comprises a FKBP binding site;
(b) B is a moiety which is a cancer binding moiety (“CBM” or “PBM”), often a prostate-specific membrane antigen (PSMA) and which has the formula:

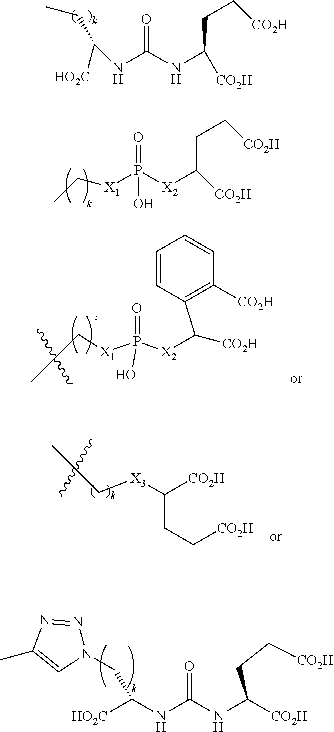

where X₁ and X₂ are each independently CH₂, O, NH or S;
X₃ is O, CH₂, NR¹, S(O), S(O)₂, —S(O)₂O, —OS(O)₂, or OS(O)₂O;
R¹ is H, a C₁-C₃ alkyl group, or a —C(O)(C₁-C₃) group;
k is an integer from Oto 20, 8 to 12, 1 to 15, 1 to 10, 1 to 8, 1 to 6, 1, 2, 3, 4, 5 or 6, or a pharmaceutically acceptable salt and/or stereoisomer; and
(c) L is a linker as otherwise described herein, preferably a linker according to the chemical formula:

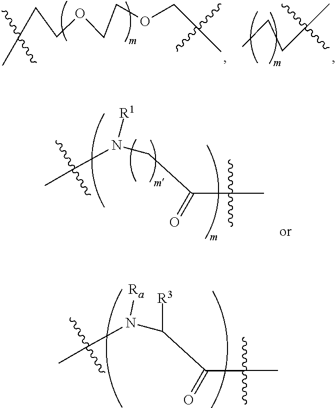

Where R¹ is H or a C1-C₃ alkyl group;
Rₐ is H, C₁-C₃ alkyl or alkanol or forms a cyclic ring with R³ to form a proline or hydroxyproline unit and R³ is a side chain derived from an amino acid preferably selected from the group consisting of alanine (methyl), arginine (propyleneguanidine), asparagine (methylenecarboxyamide), aspartic acid (ethanoic acid), cysteine (thiol, reduced or oxidized di-thiol), glutamine (ethylcarboxyamide), glutamic acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline or hydroxyproline (such that R³ forms a cyclic ring with Rₐ and the adjacent nitrogen group to form a pyrrolidine or hydroxypyrrolidine group), serine (methanol), threonine (ethanol, 1-hydroxyethane), tryptophan (methyleneindole), tyrosine (methylene phenol) or valine (isopropyl);
m′ is an integer from Oto 20, 1 to 15, 1 to 12, 1 to 9, 2 to 8, 2-4, or 5-8, often 6 or 7; each m (within this context) is independently an integer from 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5, or L is a polyethylene glycol, polypropylene glycol or polypropylene-co-polyethylene glycol linker having between 1 and 100 glycol units (1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 52 and 50, 3 and 45); and
(d) CON is a bond or is a connector moiety selected from the group consisting of:

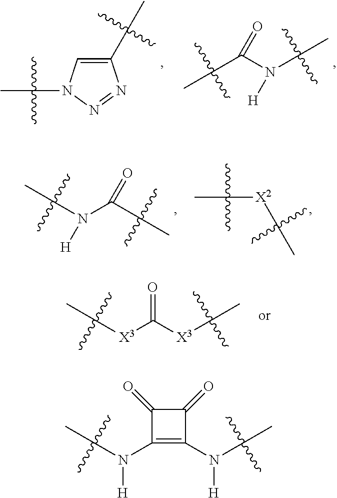

where X² is O, S, NR⁴, S(O), S(O)₂, —S(O)₂O, —OS(O)₂, or OS(O)₂O;
X³ is O, S, NR⁴; and
R⁴ is H, a C₁-C₃ alkyl or alkanol group, or a —C(O)(C₁-C₃) group, or a pharmaceutically acceptable salt, solvate, polymorph or stereoisomer thereof.

In one preferred embodiment, the invention provides chimeric antigen receptor (CAR) T cells wherein:
(a) the antigen binding domain of the chimeric antigen receptor (CAR) T cell comprises a dehalogenase (halotag) protein and the bi-functional molecule has the formula:

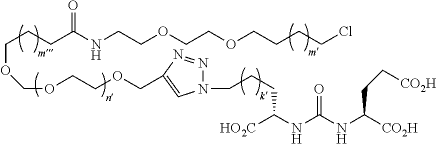

Where k′ is 0-6, preferably 1-6, often 2-4, more preferably 2;
n′ is 0-20, often 1-15, 1-12, more preferably 2-8, often 6, 7 or 8;
m′ is from 0-5, preferably 1-4, more preferably 2-4, more preferably 3; and
m′″ is from 0-5, preferably O, 1 or 2, or a pharmaceutically acceptable salt or stereoisomer thereof.

In alternative embodiments, the bi-functional molecule has the chemical structure:

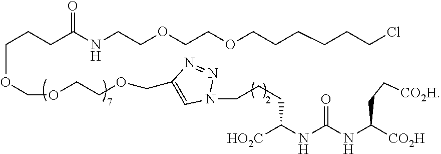

In another embodiment, the invention provides chimeric antigen receptor (CAR) T cells wherein the antigen binding domain of the chimeric antigen receptor (CAR) T cell is a snaptag protein and the bi-functional molecule has the formula:

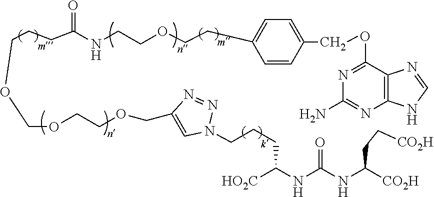

Where k′ is 0-6, preferably 1-6, preferably 2-4, more preferably 2;
n′ (in this context) is 0-20, often 1-15, 1-12, 8-12, 2-8, often 1, 2, 3, 4, 5, 6, 7, 10, 11 or 12;
n″ is 0-20, 1-16, preferably 0-8, more preferably 0-6, often 2, 3, 4 or 5;
m″ is from 0-5, preferably 1-4, more preferably 1, 2 or 3, more preferably 1 or 2; and
m′″ is from 0-5, preferably O, 1 or 2, or a pharmaceutically acceptable salt or stereoisomer thereof.

In preferred embodiments, k′ is 2, n′ is 5, 6, 7, 10, 11 or 12, n″ is 2, 3, 4 or 5 and m″ is 1 or 2. A preferred compound related to the above is:

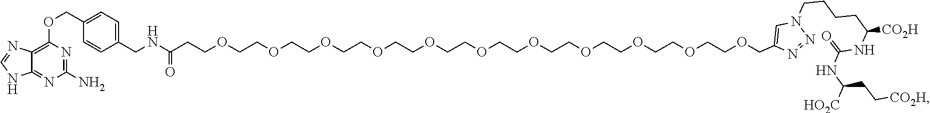 [SMILES: Nc1nc(OCc2ccc(CNC(=O)CCOCCOCCOCCOCCOCCOCCOCCOCCOCCOCCOCc3cn(CCCC[C@H](NC(=O)N[C@@H](CCC(=O)O)C(=O)O)C(=O)O)nn3)cc2)c2nc[nH]c2n1]

or
a pharmaceutically acceptable salt or stereoisomer thereof.

In another embodiment, the invention provides chimeric antigen receptor (CAR) T cells wherein the antigen binding domain of the chimeric antigen receptor (CAR) T cell is a cliptag protein specific for O2-benzyl cytosine groups and the bi-functional molecule has the formula:

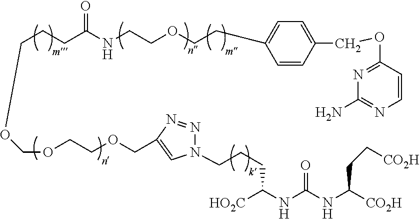

Where k′ is 0-6, preferably 1-6, preferably 2-4, more preferably 2;
n′ is 0-20, often 1-12, more preferably 2-8, often 1, 2, 3, 4, 5, 6, or 7;
n″ is 0-20, 1-16, preferably 1-8, more preferably 1-6, often 2, 3, 4 or 5;
m″ is from 0-5, preferably 1-4, more preferably 1, 2 or 3, more preferably 1 or 2; and
m′″ is 0-5, preferably O, 1 or 2,
or a pharmaceutically acceptable salt or stereoisomer thereof.

In preferred embodiments, k′ is 2, n′ is 5, 6, 7, 8, 9, 10 or 11, n″ is O, 1, 2, 3, 4 or 5 and m″ is 1 or 2.

In another embodiment, the invention provides chimeric antigen receptor (CAR) T cells wherein the antigen binding domain of the chimeric antigen receptor (CAR) T cell is FKBP12 and the bi-functional molecule has the formula:

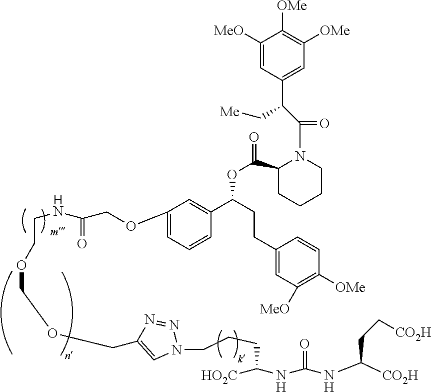

Where k′ is 0-6, preferably 1-6, preferably 2-4, more preferably 2;
n′ is 0-20, often 1-12, more preferably 2-8, often 6, 7 or 8;
m′″ is 0-5, preferably O, 1 or 2, or
a pharmaceutically acceptable salt or stereoisomer thereof.

In one embodiment, the bifunctional compound has the following chemical structure:

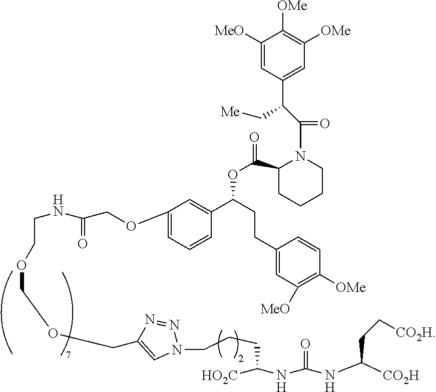

In another embodiment, the invention provides an isolated nucleic acid molecule encoding a polypeptide comprising:
(a) halotag protein, a snaptag protein, a cliptag protein or a FKBP or an amino acid sequence that exhibits substantial homology with or substantial similarity to a FKBP and that at a minimum comprises a FKBP binding site;
(b) a hinge domain;
(c) a transmembrane domain (preferably a human CD28 transmembrane domain);
(d) a co-stimulatory signaling region; and
(e) a signaling domain (preferably, a CD3 zeta (CD3-ζ) signaling domain).

Examples of each of the components of the CAR polypeptide are shown in FIG. 5 hereof, and related sequences of the vectors which express the CAR polypeptides, the sequences for the CAR polypeptides themselves as well as the components which comprise the CAR polypeptides are presented in FIGS. 16-24 hereof.

In another embodiment, the invention also provides a vector (including a retroviral vector, e.g. a gamma-retroviral or lentiviral vectors or a DNA transposon vector, among others, as described herein) comprising an isolated nucleic acid as described above, preferably operably linked to a constitutive or inducible promoter (preferably a CMV (constitutive) or other promoter).

In still another embodiment, the invention provides an isolated host cell (preferably a human T cell) that is transduced with a vector as described above. The transduced T cell comprises a CAR polypeptide as described herein in the absence of a conjugated bi-functional molecule or optionally, the CAR T cell includes a bi-functional molecule which is conjugated to the antigen binding region of the CAR polypeptide which is expressed by the T cell.

In still another embodiment, the invention provides a chimeric antigen receptor (CAR) T cell to which is conjugated a bifunctional molecule as is otherwise described herein.

In still other embodiments, the invention provides pharmaceutical compositions comprising chimeric antigen receptor (CAR) T cell-bi-functional molecule conjugates as described and claimed herein (target specific chimeric antigen receptor bearing T-cells (SMART CARs), anti-cancer methods of treatment that use theses conjugates and related diagnostic assays and kits.

In another embodiment, the bifunctional molecule conjugated chimeric antigen receptor bearing T-cells (SMART CARs), formulated for pharmaceutical delivery are administered to a patient in need for the treatment of cancer, often prostate cancer, including metastatic and/or recurrent prostate cancer. The method comprises administering an effective number of bifunctional molecule conjugated chimeric antigen receptor bearing T-cells (SMART CARs), optionally in combination with at least one additional anticancer agent, preferably an anticancer compound as described in detail herein in order to favorable treat cancer in a patient in need, often a patient suffering from prostate cancer, including metastatic or recurrent prostate cancer.

The small molecule adapter regulated, target specific chimeric antigen receptor bearing T-Cells (SMART CARs) afford many advantages over known CAR T cell designs and therapeutic regimens. For example, the SMART CARs according to the present invention are able to reduce toxicity by calibrating the immune response by varying the levels of the administered small molecule adapter intermediate. The present invention also prevents undesirable side effects caused by inappropriate sustained activation of the relevant T-Cells after completion of treatment by taking away the small molecule, thus providing temporal control over the immune response. Further, they are able to engage a single engineered T-Cell construct to multiple targets by varying only the targeting domain of the small molecule for effective combination therapy (this obviates the need for possible multiple transfusions with T-Cells against different targets, or transduction of multiple distinct CARs into a single cell product, as in current CAR technology). Finally, the SMART CARs according to the present invention facilitate customization of patient specific mixed and matched small molecules depending on the determined quantities of surface expression of targets in the patient's tumor.

These and other aspects of the invention are described further in the Detailed Description of the Invention.

### BRIEF DESCRIPTION OF THE FIGURES

FIG. 1A illustrates the cloning of the first generation SMART CAR construct which included a HaloTag® protein and its cognate, ligand-based small molecule intermediates 1B.

FIGS. 2A-B illustrate the design of the second generation SMART CAR constructs (promoter-nucleic acid sequences) Figure A) shows a comparison of the first and second generation SMART CARs and Figure B) shows the construct design and its PSMA targeting small molecule binding partner.

FIG. 3 illustrates the nucleofaction of CAR1 construct into CD4+ Jurkat T cell line and staining with antibodies directed against the surface expressed Halo protein.

FIG. 4 illustrates the stimulation of SMART CAR transfected or untransfected Jurkat T cells by Streptavidin in the presence of Biotin-HaloTag® intermediate adapter.

FIGS. 5A-B illustrate the major components of the Vectors; Figure A) CAR1, CAR 2, CAR4 and CAR 10 which represent the first three generation vectors utilizing compositions and methods according to the present invention. These vectors contain a halo protein, FKBP12 polypeptide or a snap tag as the antigen binding domain of the chimeric antigen receptor. The first and second generation vectors CAR1 and CAR2 include the hinge domain, the transmembrane domain and the co-stimulatory signaling region within the CD28 element. In each of these cases, CD3 Zeta was used as the signaling domain. In each of the third generation vectors, CAR3, CAR4 and CAR10, a second co-stimulatory region 4-1BB was included in the vector for purposes of increasing the quantity, strength of the activation, potency and memory and to influence the phenotype of the T cells, and the quantity and type of cytokines released. In the third generation, a Snap Tap antigen binding region was added to the CAR to form vector CAR 10; Figure B) illustrates certain vectors directed to CAR7 and CAR13 which contain in addition to the necessary components of the chimeric antigen receptor, additional components P2A, which is a 2A cleavage peptide and EGFRt, which is a truncated epidermal growth factor receptor (EGFR) gene (Q9H3C8)), which have been inserted into the vector to allow expression in the chimeric antigen receptor to delete cells and/or to assist in selecting cells as part of a cell purification method. The P2A peptide allows cleavage of the EGFRt from the remaining portion of the chimeric antigen receptor (CAR) after translation.

FIG. 6 shows that primary human SMART CAR T cells according to the present invention are activated in the presence of adaptor and target cells.

FIG. 7 shows that SMART CAR T cell co-incubation with adaptor and target cells induces activation and IL-2 production in a dose-dependent manner.

FIG. 8 shows that SMART CAR T cells according to the present invention lyse target cells in a dose-dependent manner.

FIG. 9 shows that SMART CAR T cells according to the present invention activate, produce cytokines and kill cells (cytotoxicity) in a dose-dependent manner.

FIG. 10 shows a direct comparison of SnapTag and HaloTag CAR activation through bifunctional-molecule engagement, demonstrating similar levels of activation for each.

FIG. 11 shows that fusing a truncated epidermal growth factor receptor (EGFRt) on the CAR can provide useful information as a binding site for incorporation into an assay. In FIG. 11, up to about 40% of the CAR+ cells were strongly activated (left panel) and approximately 75% of CAR+ cells are CD69+ cells.

FIG. 12 shows that the addition of EGFRt to the chimeric antigen receptor (CAR) appears to have reduced expression and activation of the CAR comprising the EGFRt compared to CAR which does not comprise EGFRt.

FIG. 13 shows that there is little apparent variation between the SMART CARs based on the PSMA expression levels which were identified.

FIG. 14 evidences that EGFTt bead positive selection is an effective selective method.

FIG. 15 is directed to the amino acid sequences for the halotag polypeptide (halotag 2 and halotag 7, SEQ ID NO: 1 and SEQ ID NO:2), snaptag polypeptide (psnap-tag(m), psnap-tag(m)2, psnap-tag(T7) and psnap-tag(T7)2, SEQ ID NO: 3, SEQ ID NO:4, SEQ ID NO:5 and SEQ ID NO:6) and cliptag polypeptide (pclip-tag(m), SEQ ID NO: 7).

FIG. 16 is directed to the DNA sequence for the CAR 1 Vector PLVX CAR1 SEQUENCE (SEQ ID NO: 31) which encodes the CAR 1 polypeptide and other components of the vector as indicted in FIG. 5.

FIG. 17 is directed to the DNA sequence for the CAR 2 Vector PLVX CAR2 SEQUENCE (SEQ ID NO: 32) which encodes the CAR 2 polypeptide and other components of the vector as indicted in FIG. 5.

FIG. 18 is directed to the DNA sequence for the CAR 3 Vector PLVX CAR3 SEQUENCE (SEQ ID NO: 33) which encodes the CAR 3 polypeptide and other components of the vector as indicted in FIG. 5.

FIG. 19 is directed to the DNA sequence for the CAR 4 Vector PLVX CAR4 SEQUENCE (SEQ ID NO: 34) which encodes the CAR 4 polypeptide and other components of the vector as indicted in FIG. 5.

FIG. 20 is directed to the DNA sequence for the CAR 7 Vector PLVX CAR7 SEQUENCE (SEQ ID NO: 35) which encodes the CAR 7 polypeptide and other components of the vector as indicted in FIG. 5.

FIG. 21 is directed to the DNA sequence for the CAR 10 Vector PLVX CAR10 SEQUENCE (SEQ ID NO: 36) which encodes the CAR 10 polypeptide and other components of the vector as indicted in FIG. 5.

FIG. 22 is directed to the DNA sequence for the CAR 13 Vector PLVX CAR13 SEQUENCE (SEQ ID NO: 37) which encodes the CAR 13 polypeptide and other components of the vector as indicted in FIG. 5.

FIG. 23 is directed to the DNA Sequences which encode for each of the CAR polypeptides which are presented for each of the vectors presented in FIG. 5. The DNA sequence which encodes for the CAR1 polypeptide is SEQ ID NO: 38; for CAR2 the sequence is SEQ ID NO: 39; for CAR3 the sequence is SEQ ID NO: 40; for CAR4 the sequence is SEQ ID NO:41; for CAR7 the sequence is SEQ ID No: 42; for CAR10 the sequence is SEQ ID NO: 43; and for CAR13 the sequence is SEQ ID NO: 44.

FIG. 24 is directed to DNA sequences which encode for the individual components as indicated which comprise the various CAR polypeptides which are presented in FIG. 5 hereof.

FIG. 25 is directed to a group of moieties which can be used to bind to bi-functional molecules to CAR polypeptides which comprise antigen binding regions of FKBP family of proteins. The moieties represented are FK506 (tacrolimus), a FK506 derivative or a rapalog, more specifically moieties of tacrolimus (FK506), FK1706, meridamycin, normeridamycin, ILS920, Way-124466, Wye-592, L685-818, VX-10,367, VX-710 (Biricodar), VX-853 (Timcodar), JNJ460/GM284, GPI1046, GPI1485 and DM-CHX; useful rapologs include but are not limited to rapamycin (sirolimus), temsirolimus (CCI 779), everolimus (RAD001) and ridaforolimus/deforolimus (AP-23573). It is noted that in certain instances, the depicted moiety has more than one attachment point X, as noted. In the structures of this figure X is O, C═O, CH₂, NR¹, C(O)NR¹, NR¹C(O), S(O), S(O)₂, —S(O)₂O, —OS(O)₂, or OS(O)₂O (preferably, O, C═O, CH₂, NR¹, C(O)NR¹, NR¹C(O)), where R¹ is H or a C₁-C₃ alkyl, preferably H, such that the attachment point and the moiety produce a chemically stable bond. It is noted that in embodiments, only one attachment point is used to covalently bind the moiety to the bi-functional molecule as described herein and where more than on attachment group is depicted that moiety can bind at any of those attachment points to produce a bi-functional molecule as described herein.

### DETAILED DESCRIPTION OF THE INVENTION

The following terms are used to describe the present invention. In instances where a term is not specifically defined herein, that term is given an art-recognized meaning by those of ordinary skill applying that term in context to its use in describing the present invention.

The term “compound” or “molecule”, as used herein, unless otherwise indicated, refers to any specific chemical compound disclosed herein and includes tautomers, regioisomers, geometric isomers, and where applicable, optical isomers (enantiomers) thereof, as well as pharmaceutically acceptable salts and derivatives (including prodrug forms) thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents, linkers and connector molecules and variables associated with same, among others, are described. It is understood by those of ordinary skill that molecules which are described herein are stable compounds as generally described hereunder.

The term “patient” or “subject” is used throughout the specification within context to describe an animal, generally a mammal and preferably a human, to whom treatment, including prophylactic treatment (prophylaxis), with the compositions according to the present invention is provided. For treatment of those infections, conditions or disease states which are specific for a specific animal such as a human patient or a patient of a particular gender, such as a human male patient, the term patient refers to that specific animal. Chimeric antigen receptor (CAR) T cell-bi-functional molecule conjugates according to the present invention are useful for the treatment of cancer, especially including prostate cancer and in particular, metastatic prostate cancer.

The term “effective” is used herein, unless otherwise indicated, to describe an amount of a compound or composition which, in context, is used to produce or effect an intended result, whether that result relates to the inhibition of the effects of a toxicant on a subject or the treatment of a subject for secondary conditions, disease states or manifestations of exposure to toxicants as otherwise described herein. This term subsumes all other effective amount or effective concentration terms (including the term “therapeutically effective”) which are otherwise described in the present application.

The terms “treat”, “treating”, and “treatment”, etc., as used herein, refer to any action providing a benefit to a patient at risk for prostate cancer or metastasis of prostate cancer, including improvement in the condition through lessening or suppression of at least one symptom, inhibition of cancer growth, reduction in cancer cells or tissue, prevention or delay in progression of metastasis of the cancer, prevention or delay in the onset of disease states or conditions which occur secondary to cancer or remission or cure of the cancer, among others. Treatment, as used herein, encompasses both therapeutic treatment and prophylactic treatment where appropriate within the context of its use. The term “prophylactic” when used, means to reduce the likelihood of an occurrence or the severity of an occurrence within the context of the treatment of cancer, including cancer metastasis as otherwise described hereinabove.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Representative cancers include, for example, prostate cancer, metastatic prostate cancer, recurrent prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more chimeric antigen receptor (CAR) T cell-bi-functional molecule conjugates according to the present invention. Because of the activity of the present chimeric antigen receptor (CAR) T cell-bi-functional molecule conjugates as anti-angiogenic compounds, the present invention has general applicability treating virtually any cancer in any tissue, thus the compounds, compositions and methods of the present invention are generally applicable to the treatment of cancer. In preferred embodiments, the cancer to be treated is a cancer which overexpresses or hyperexpresses PSMA, often prostate cancer, metastatic and/or recurrent prostate cancer. Given that the protein target is found on the neovasculature of most non-prostatic cancer cells, the compounds in the present invention may also serve as an antiangiogenic therapy or as ancillary antiangiogenic therapy for other cancer types.

In certain particular aspects of the present invention, the cancer which is treated is prostate cancer or metastatic prostate cancer. Separately, metastatic prostate cancer may be found in virtually all tissues of a cancer patient in late stages of the disease, typically metastatic prostate cancer is found in seminal vesicles, lymph system/nodes (lymphoma), in bones, in bladder tissue, in kidney tissue, liver tissue and in virtually any tissue, including brain (brain cancer/tumor). Thus, the present invention is generally applicable and may be used to treat any cancer in any tissue, regardless of etiology.

The term “prostate cancer” is used to describe a disease in which cancer develops in the prostate, a gland in the male reproductive system. It occurs when cells of the prostate mutate and begin to multiply uncontrollably. These cells may metastasize (metastatic prostate cancer) from the prostate to virtually any other part of the body, particularly the bones and lymph nodes, but the kidney, bladder and even the brain, among other tissues. Prostate cancer may cause pain, difficulty in urinating, problems during sexual intercourse, erectile dysfunction. Other symptoms can potentially develop during later stages of the disease.

Rates of detection of prostate cancers vary widely across the world, with South and East Asia detecting less frequently than in Europe, and especially the United States. Prostate cancer develops most frequently in men over the age of fifty and is one of the most prevalent types of cancer in men. However, many men who develop prostate cancer never have symptoms, undergo no therapy, and eventually die of other causes. This is because cancer of the prostate is, in most cases, slow-growing, and because most of those affected are over the age of 60. Hence, they often die of causes unrelated to the prostate cancer. Many factors, including genetics and diet, have been implicated in the development of prostate cancer. The presence of prostate cancer may be indicated by symptoms, physical examination, prostate specific antigen (PSA), or biopsy. There is concern about the accuracy of the PSA test and its usefulness in screening. Suspected prostate cancer is typically confirmed by taking a biopsy of the prostate and examining it under a microscope. Further tests, such as CT scans and bone scans, may be performed to determine whether prostate cancer has spread.

Treatment options for prostate cancer with intent to cure are primarily surgery and radiation therapy. Other treatments such as hormonal therapy, chemotherapy, proton therapy, cryosurgery, high intensity focused ultrasound (HIFU) also exist depending on the clinical scenario and desired outcome.

The age and underlying health of the man, the extent of metastasis, appearance under the microscope, and response of the cancer to initial treatment are important in determining the outcome of the disease. The decision whether or not to treat localized prostate cancer (a tumor that is contained within the prostate) with curative intent is a patient trade-off between the expected beneficial and harmful effects in terms of patient survival and quality of life.

An important part of evaluating prostate cancer is determining the stage, or how far the cancer has spread. Knowing the stage helps define prognosis and is useful when selecting therapies. The most common system is the four-stage TNM system (abbreviated from Tumor/Nodes/Metastases). Its components include the size of the tumor, the number of involved lymph nodes, and the presence of any other metastases.

The most important distinction made by any staging system is whether or not the cancer is still confined to the prostate or is metastatic. In the TNM system, clinical T1 and T2 cancers are found only in the prostate, while T3 and T4 cancers have spread elsewhere and metastasized into other tissue. Several tests can be used to look for evidence of spread. These include computed tomography to evaluate spread within the pelvis, bone scans to look for spread to the bones, and endorectal coil magnetic resonance imaging to closely evaluate the prostatic capsule and the seminal vesicles. Bone scans often reveal osteoblastic appearance due to increased bone density in the areas of bone metastasis—opposite to what is found in many other cancers that metastasize. Computed tomography (CT) and magnetic resonance imaging (MRI) currently do not add any significant information in the assessment of possible lymph node metastases in patients with prostate cancer according to a meta-analysis.

Prostate cancer is relatively easy to treat if found early. After a prostate biopsy, a pathologist looks at the samples under a microscope. If cancer is present, the pathologist reports the grade of the tumor. The grade tells how much the tumor tissue differs from normal prostate tissue and suggests how fast the tumor is likely to grow. The Gleason system is used to grade prostate tumors from 2 to 10, where a Gleason score of 10 indicates the most abnormalities. The pathologist assigns a number from 1 to 5 for the most common pattern observed under the microscope, then does the same for the second most common pattern. The sum of these two numbers is the Gleason score. The Whitmore-Jewett stage is another method sometimes used. Proper grading of the tumor is critical, since the grade of the tumor is one of the major factors used to determine the treatment recommendation.

Early prostate cancer usually causes no symptoms. Often it is diagnosed during the workup for an elevated PSA noticed during a routine checkup. Sometimes, however, prostate cancer does cause symptoms, often similar to those of diseases such as benign prostatic hypertrophy. These include frequent urination, increased urination at night, difficulty starting and maintaining a steady stream of urine, blood in the urine, and painful urination. Prostate cancer is associated with urinary dysfunction as the prostate gland surrounds the prostatic urethra. Changes within the gland therefore directly affect urinary function. Because the vas deferens deposits seminal fluid into the prostatic urethra, and secretions from the prostate gland itself are included in semen content, prostate cancer may also cause problems with sexual function and performance, such as difficulty achieving erection or painful ejaculation.

Advanced prostate cancer can spread to other parts of the body and this may cause additional symptoms. The most common symptom is bone pain, often in the vertebrae (bones of the spine), pelvis or ribs. Spread of cancer into other bones such as the femur is usually to the proximal part of the bone. Prostate cancer in the spine can also compress the spinal cord, causing leg weakness and urinary and fecal incontinence.

The specific causes of prostate cancer remain unknown. A man's risk of developing prostate cancer is related to his age, genetics, race, diet, lifestyle, medications, and other factors. The primary risk factor is age. Prostate cancer is uncommon in men less than 45, but becomes more common with advancing age. The average age at the time of diagnosis is 70. However, many men never know they have prostate cancer.

A man's genetic background contributes to his risk of developing prostate cancer. This is suggested by an increased incidence of prostate cancer found in certain racial groups, in identical twins of men with prostate cancer, and in men with certain genes. Men who have a brother or father with prostate cancer have twice the usual risk of developing prostate cancer. Studies of twins in Scandinavia suggest that forty percent of prostate cancer risk can be explained by inherited factors. However, no single gene is responsible for prostate cancer; many different genes have been implicated. Two genes (BRCA1 and BRCA2) that are important risk factors for ovarian cancer and breast cancer in women have also been implicated in prostate cancer.

Dietary amounts of certain foods, vitamins, and minerals can contribute to prostate cancer risk. Dietary factors that may increase prostate cancer risk include low intake of vitamin E, the mineral selenium, green tea and vitamin D. A large study has implicated dairy, specifically low-fat milk and other dairy products to which vitamin A palmitate has been added. This form of synthetic vitamin A has been linked to prostate cancer because it reacts with zinc and protein to form an unabsorbable complex. Prostate cancer has also been linked to the inclusion of bovine somatotropin hormone in certain dairy products.

There are also some links between prostate cancer and medications, medical procedures, and medical conditions. Daily use of anti-inflammatory medicines such as aspirin, ibuprofen, or naproxen may decrease prostate cancer risk. Use of the cholesterol-lowering drugs known as the statins may also decrease prostate cancer risk. Infection or inflammation of the prostate (prostatitis) may increase the chance for prostate cancer, and infection with the sexually transmitted infections chlamydia, gonorrhea, or syphilis seems to increase risk. Obesity and elevated blood levels of testosterone may increase the risk for prostate cancer.

In prostate cancer, the regular glands of the normal prostate are replaced by irregular glands and clumps of cells. When a man has symptoms of prostate cancer, or a screening test indicates an increased risk for cancer, more invasive evaluation is offered. The only test which can fully confirm the diagnosis of prostate cancer is a biopsy, the removal of small pieces of the prostate for microscopic examination. However, prior to a biopsy, several other tools may be used to gather more information about the prostate and the urinary tract. Cystoscopy shows the urinary tract from inside the bladder, using a thin, flexible camera tube inserted down the urethra. Transrectal ultrasonography creates a picture of the prostate using sound waves from a probe in the rectum.

After biopsy, the tissue samples are then examined under a microscope to determine whether cancer cells are present, and to evaluate the microscopic features (or Gleason score) of any cancer found. In addition, tissue samples may be stained for the presence of PSA and other tumor markers in order to determine the origin of maligant cells that have metastasized. A number of other potential approaches for diagnosis of prostate cancer are ongoing such as early prostate cancer antigen-2 (EPCA-2), and prostasome analysis.

In addition to therapy using the chimeric antigen receptor (CAR) T cell-bi-functional molecule conjugates according to the present invention, therapy (including prophylactic therapy) for prostate cancer supports roles in reducing prostate cancer for dietary selenium, vitamin E, lycopene, soy foods, vitamin D, green tea, omega-3 fatty acids and phytoestrogens. The selective estrogen receptor modulator drug toremifene has shown promise in early trials. Two medications which block the conversion of testosterone to dihydrotestosterone (and reduce the tendency toward cell growth), finasteride and dutasteride, are shown to be useful. The phytochemicals indole-3-carbinol and diindolylmethane, found in cruciferous vegetables (califlower and broccholi), have favorable antiandrogenic and immune modulating properties. Prostate cancer risk is decreased in a vegetarian diet.

Treatment for prostate cancer may involve active surveillance, surgery (prostatecomy or orchiectomy), radiation therapy including brachytherapy (prostate brachytherapy) and external beam radiation as well as hormonal therapy. There are several forms of hormonal therapy which include the following, each of which may be combined with chimeric antigen receptor (CAR) T cell-bi-functional molecule conjugates according to the present invention.
Antiandrogens such as flutamide, bicalutamide, nilutamide, and cyproterone acetate which directly block the actions of testosterone and DHT within prostate cancer cells.
Medications such as ketoconazole and aminoglutethimide which block the production of adrenal androgens such as DHEA. These medications are generally used only in combination with other methods that can block the 95% of androgens made by the testicles. These combined methods are called total androgen blockade (TAB), which can also be achieved using antiandrogens.
GnRH modulators, including agonists and antagonists. GnRH antagonists suppress the production of LH directly, while GnRH agonists suppress LH through the process of downregulation after an initial stimulation effect. Abarelix is an example of a GnRH antagonist, while the GnRH agonists include leuprolide, goserelin, triptorelin, and buserelin.
The use of abiraterone acetate can be used to reduce PSA levels and tumor sizes in aggressive end-stage prostate cancer for as high as 70% of patients. Sorafenib may also be used to treat metastatic prostate cancer.

Each treatment described above has disadvantages which limit its use in certain circumstances. GnRH agonists eventually cause the same side effects as orchiectomy but may cause worse symptoms at the beginning of treatment. When GnRH agonists are first used, testosterone surges can lead to increased bone pain from metastatic cancer, so antiandrogens or abarelix are often added to blunt these side effects. Estrogens are not commonly used because they increase the risk for cardiovascular disease and blood clots. The antiandrogens do not generally cause impotence and usually cause less loss of bone and muscle mass. Ketoconazole can cause liver damage with prolonged use, and aminoglutethimide can cause skin rashes.

Palliative care for advanced stage prostate cancer focuses on extending life and relieving the symptoms of metastatic disease. As noted above, abiraterone acetate shows some promise in treating advance stage prostate cancer as does sorafenib. Chemotherapy may be offered to slow disease progression and postpone symptoms. The most commonly used regimen combines the chemotherapeutic drug docetaxel with a corticosteroid such as prednisone. Bisphosphonates such as zoledronic acid have been shown to delay skeletal complications such as fractures or the need for radiation therapy in patients with hormone-refractory metastatic prostate cancer. Alpharadin may be used to target bone metastasis. The phase II testing shows prolonged patient survival times, reduced pain and improved quality of life.

Bone pain due to metastatic disease is treated with opioid pain relievers such as morphine and oxycodone. External beam radiation therapy directed at bone metastases may provide pain relief Injections of certain radioisotopes, such as strontium-89, phosphorus-32, or samarium-153, also target bone metastases and may help relieve pain.

As an alternative to active surveillance or definitive treatments, alternative therapies may also be used for the management of prostate cancer. PSA has been shown to be lowered in men with apparent localized prostate cancer using a vegan diet (fish allowed), regular exercise, and stress reduction. Many other single agents have been shown to reduce PSA, slow PSA doubling times, or have similar effects on secondary markers in men with localized cancer in short term trials, such as pomegranate juice or genistein, an isoflavone found in various legumes.

Manifestations or secondary conditions or effects of metastatic and advanced prostate cancer may include anemia, bone marrow suppression, weight loss, pathologic fractures, spinal cord compression, pain, hematuria, ureteral and/or bladder outlet obstruction, urinary retention, chronic renal failure, urinary incontinence, and symptoms related to bony or soft-tissue metastases, among others.
Additional prostate drugs which can be used in combination with the chimeric antibody recruiting compounds according to the present invention include, for example, the enlarged prostate drugs/agents, as well as eulexin, flutamide, goserelin, leuprolide, lupron, nilandron, nilutamide, zoladex and mixtures thereof. Enlarged prostate drugs/agents as above, include for example, ambenyl, ambophen, amgenal, atrosept, bromanyl, bromodiphenhydramine-codeine, bromotuss-codeine, cardura, chlorpheniramine-hydrocodone, ciclopirox, clotrimazole-betamethasone, dolsed, dutasteride, finasteride, flomax, gecil, hexalol, lamisil, lanased, loprox, lotrisone, methenamine, methen-bella-meth Bl-phen sal, meth-hyos-atrp-M blue-BA-phsal, MHP-A, mybanil, prosed/DS, Ro-Sed, S-T Forte, tamsulosin, terbinafine, trac, tussionex, ty-methate, uramine, uratin, uretron, uridon, uro-ves, urstat, usept and mixtures thereof.

The term “tumor” is used to describe a malignant or benign growth or tumefacent.

“Hydrocarbon” or “hydrocarbyl” refers to any monovalent (or divalent in the case of alkylene groups) radical containing carbon and hydrogen, which may be straight, branch-chained or cyclic in nature. Hydrocarbons include linear, branched and cyclic hydrocarbons, including alkyl groups, alkylene groups, saturated and unsaturated hydrocarbon groups including aromatic groups both substituted and unsubstituted, alkene groups (containing double bonds between two carbon atoms) and alkyne groups (containing triple bonds between two carbon atoms). In certain instances, the terms substituted alkyl and alkylene are sometimes used synonymously.

“Alkyl” refers to a fully saturated monovalent radical containing carbon and hydrogen, and which may be cyclic, branched or a straight chain. Examples of alkyl groups are methyl, ethyl, n-butyl, n-hexyl, n-heptyl, n-octyl, n-nonyl, n-decyl, isopropyl, 2-methyl-propyl, cyclopropyl, cyclopropylmethyl, cyclobutyl, cyclopentyl, cyclopentylethyl, cyclohexylethyl and cyclohexyl. Preferred alkyl groups are C₁-C₆ alkyl groups. “Alkylene” refers to a fully saturated hydrocarbon which is divalent (may be linear, branched or cyclic) and which is optionally substituted. Preferred alkylene groups are C₁-C₆ alkylene groups. Other terms used to indicate substitutent groups in compounds according to the present invention are as conventionally used in the art.

The term “aryl” or “aromatic”, in context, refers to a substituted or unsubstituted monovalent aromatic radical having a single ring (e.g., benzene, benzyl or phenyl). Other examples of aryl groups, in context, may include heterocyclic aromatic ring systems “heteroaryl” groups having one or more nitrogen, oxygen, or sulfur atoms in the ring (5- or 6-membered heterocyclic rings) such as imidazole, furyl, pyrrole, pyridyl, furanyl, thiene, thiazole, pyridine, pyrimidine, pyrazine, triazole, oxazole, among others, which may be substituted or unsubstituted as otherwise described herein.

The term “heterocyclic group” “heterocycle” as used throughout the present specification refers to an aromatic (“heteroaryl”) or non-aromatic cyclic group forming the cyclic ring and including at least one and up to three hetero atoms such as nitrogen, sulfur or oxygen among the atoms forming the cyclic ring. The heterocyclic ring may be saturated (heterocyclic) or unsaturated (heteroaryl). Exemplary heterocyclic groups include, for example pyrrolidinyl, piperidinyl, morpholinyl, pyrrole, pyridine, pyridone, pyrimidine, imidazole, thiophene, furan, pyran, thiazole, more preferably pyrimidinyl, pyrrolidinyl, piperidinyl, morpholinyl, oxazole, isoxazole, pyrrole, pyridine, thiophene, thiazole and even more preferably pyrimidinyl, especially uracil or cytosine which are optionally substituted, furyl, 3-methylfuryl, thiazole, piperazinyl, N-methylpiperazinyl, tetrahydropyranyl and 1,4-dioxane, among others. Additional heterocyclic groups include oxazole, benzoxazole, pyrrole, dihydropyrrole, benzopyrrole, benzodihydropyrrole, indole, indolizine, among others.

Exemplary heteroaryl moieties which may be used in the present invention include for example, pyrrole, pyridine, pyridone, pyridazine, pyrimidine, pyrazine, pyrazole, imidazole, triazole, tetrazole, oxadiazole, sulfur-containing aromatic heterocycles such as thiophene; oxygen-containing aromatic heterocycles such as furan and pyran, and including aromatic heterocycles comprising 2 or more hetero atoms selected from among nitrogen, sulfur and oxygen, such as thiazole, thiadiazole, isothiazole, isoxazole, furazan and oxazole. Further heteroaryl groups may include pyridine, triazine, pyridone, pyrimidine, imidazole, furan, pyran, thiazole. Pyrimidine groups, especially uracil and cytosine, optionally substituted, are preferred.

The term “substituted” shall mean substituted at a carbon (or nitrogen) position within context, hydroxyl, carboxyl, cyano (C═N), nitro (NO₂), halogen (preferably, 1, 2 or 3 halogens, especially on an alkyl, especially a methyl group such as a trifluoromethyl), alkyl group (preferably, C₁-C₁₀, more preferably, C₁-C₆), alkoxy group (preferably, C₁-C₆ alkyl or aryl, including phenyl and substituted phenyl), ester (preferably, C₁-C₆alkyl or aryl) including alkylene ester (such that attachment is on the alkylene group, rather than at the ester function which is preferably substituted with a C₁-C₆ alkyl or aryl group), preferably, C₁-C₆ alkyl or aryl, halogen (preferably, F or Cl), nitro or amine (including a five- or six-membered cyclic alkylene amine, further including a C₁-C₆ alkyl amine or C₁-C₆ dialkyl amine which alkyl groups may be substituted with one or two hydroxyl groups), amido, which is preferably substituted with one or two C₁-C₆ alkyl groups (including a carboxamide which is substituted with one or two C₁-C₆ alkyl groups), alkanol (preferably, C₁-C₆ alkyl or aryl), or alkanoic acid (preferably, C₁-C₆ alkyl or aryl). Preferably, the term “substituted” shall mean within its context of use alkyl, alkoxy, halogen, ester, keto, nitro, cyano and amine (especially including mono- or di-C₁-C₆ alkyl substituted amines which may be optionally substituted with one or two hydroxyl groups). Any substitutable position in a compound according to the present invention may be substituted in the present invention, but no more than 3, more preferably no more than 2 substituents (in some instances only 1 or no substituents) is present on a ring. Preferably, the term “unsubstituted” shall mean substituted with one or more H atoms.

“Halogen” or “halo” may be fluoro, chloro, bromo or iodo. In preferred embodiments, especially alkyl halides, the halogen is a chloro group.

The term “linker” is used to describe a chemical entity connecting a moiety which binds to the antigen binding domain of the chimeric antigen receptor (CAR) T cell (A) (“CAR T cell binding moiety” or CARBM) and a moiety which binds to a prostate-specific membrane antigen (PSMA) (B) (“cancer binding moiety” or CBM or PBM), optionally through a connector moiety (CON) through covalent bonds. The linker between the two active portions of the molecule, that is the CAR T cell binding moiety (CARBM) and the cancer binding moiety (PBM) ranges from about 5 Å to about 50 Å or more in length, about 6 Å to about 45 Å in length, about 7 Å to about 40 Å in length, about 8 Å to about 35 Å in length, about 9 Å to about 30 Å in length, about 10 Å to about 25 Å in length, about 7 Å to about 20 Å in length, about 5 Å to about 16 Å in length, about 5 Å to about 15 Å in length, about 6 Å to about 14 Å in length, about 10 to about 20 Å in length, about 11 to about 25 Å in length, etc. Linkers which are based upon ethylene glycol units and are between 4 and 14 glycol units in length may be preferred. By having a linker with a length as otherwise disclosed herein, the CARBM moiety and the PBM moiety may be situated to advantageously take advantage of the biological activity of compounds according to the present invention which bind to cancer cells through the PBM moiety and attract CAR T to the cancer cells to which the compounds are bound, resulting in the selective and targeted death of those cells. The selection of a linker component is based on its documented properties of biocompatibility, solubility in aqueous and organic media, and low immunogenicity/antigenicity. Although numerous linkers may be used as otherwise described herein, a linker based upon polyethyleneglycol (PEG) linkages, polypropylene glycol linkages, or polyethyleneglycol-co-polypropylene oligomers (up to about 100 units, about 1 to 100, about 1 to 75, about 1 to 60, about 1 to 50, about 1 to 35, about 1 to 25, about 1 to 20, about 1 to 15, 2 to 10, about 4 to 12, about 1 to 8, 1 to 3, 1 to 4, 2 to 6, 1 to 5, etc.) may be favored as a linker because of the chemical and biological characteristics of these molecules. The use of polyethylene (PEG) linkages or PEG containing linkages is preferred. Alternative preferred linkers may include, for example, polyproline linkers and/or collagen linkers as depicted below (n is about 1 to 100, about 1 to 75, about 1 to 60, about 1 to 50, about 1 to 45, about 1 to 35, about 1 to 25, about 1 to 20, about 1 to 15, 2 to 10, about 4 to 12, about 5 to 10, about 4 to 6, about 1 to 8, about 1 to 6, about 1 to 5, about 1 to 4, about 1 to 3, etc.). As disclosed a linker group may optionally comprise a connector (CON) group or another group which technically bridges a linker to another portion of the molecule. These groups include amide groups, amine groups, alkylene groups (e.g., a C₁-C₁₀ alkylene group), a urethane group and CON groups as otherwise disclosed herein.

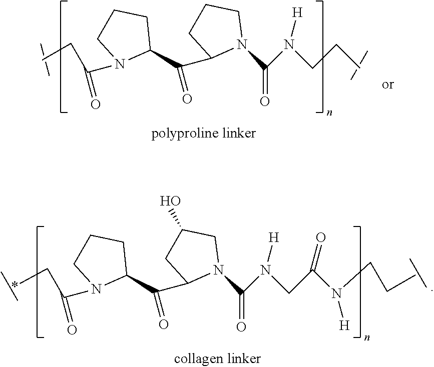

Preferred linkers include those according to the chemical structures:

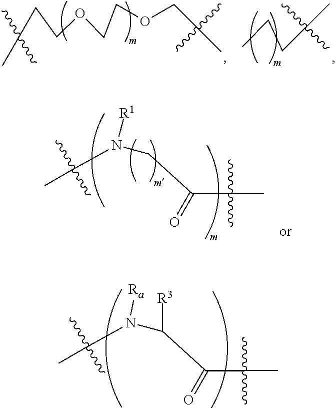

Or a polyethylene glycol, polypropylene glycol or polypropylene-co-polyethylene glycol linker having between 1 and 100 glycol units;
Where R¹ is H or a C₁-C₃ alkyl group;
Rₐ is H, C₁-C₃ alkyl or alkanol or forms a cyclic ring with R³ to form proline or hydroxyproline and R³ is a side chain derived from an amino acid preferably selected from the group consisting of alanine (methyl), arginine (propyleneguanidine), asparagine (methylenecarboxyamide), aspartic acid (ethanoic acid), cysteine (thiol, reduced or oxidized di-thiol), glutamine (ethylcarboxyamide), glutamic acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline or hydroxyproline (R³ forms a cyclic ring with Rₐ and the adjacent nitrogen group to form a pyrrolidine or hydroxyproline group), serine (methanol), threonine (ethanol, 1-hydroxyethane), tryptophan (methyleneindole), tyrosine (methylene phenol) or valine (isopropyl);
m′ is 0 to 15, 1 to 12, 1 to 9, 2 to 8, 2-4, or 5-8;
each m (within this context) is independently an integer from 1 to 100, 1 to 75, 1 to 60, 1 to 55, 1 to 50, 1 to 45, 1 to 40, 2 to 35, 3 to 30, 1 to 15, ito 10, 1 to 8, 1 to 6, 1, 2, 3, 4 or 5; n (within this context) is an integer from about 1 to 100, about 1 to 75, about 1 to 60, about 1 to 50, about 1 to 45, about 1 to 35, about 1 to 25, about 1 to 20, about 1 to 15, 2 to 10, about 4 to 12, about 5 to 10, about 4 to 6, about 1 to 8, about 1 to 6, about 1 to 5, about 1 to 4, about 1 to 3, etc.) or

Another linker according to the present invention comprises a polyethylene glycol linker containing linker containing from 1 to 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5 ethylene glycol units, to which is bonded a lysine group (preferably at its carboxylic acid moiety) which binds one or two CARBM groups to the lysine at the amino group(s) of lysine. Still other linkers comprise amino acid residues (D or L) to which are bonded to CARBM moieties at various places on amino acid residues as otherwise described herein. In another embodiment, as otherwise described herein, the amino acid has anywhere from 1-15 methylene groups separating the amino group from the acid group in providing a linker to the CARBM moiety.
Or another linker is according to the chemical formula:
Where Z and Z′ are each independently a bond, —(CH₂)ᵢ—O, —(CH₂)ᵢ—S, —(CH₂)ᵢ—NR,
wherein said —(CH₂)ᵢ group, if present in Z or Z′, is bonded to a connector, CARBM moiety or cancer binding PBM group;
Each R is H, or a C₁-C₃ alkyl or alkanol group;
Each R² is independently H or a C₁-C₃ alkyl group;
Each Y is independently a bond, O, S or NR;
Each i is independently 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
D is

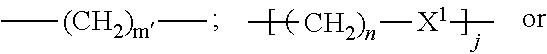

a bond, with the proviso that Z, Z′ and D are not each simultaneously bonds;
j is 1 to 100, 1 to 75, 1 to 60, 1 to 55, 1 to 50, 1 to 45, 1 to 40, 2 to 35, 3 to 30, 1 to 5, 1 to 10, 1 to 8, 1 to 6, 1, 2, 3, 4 or 5;
m′ is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
n is 1 to 100, 1 to 75, 1 to 60, 1 to 55, 1 to 50, 1 to 45, 1 to 40, 2 to 35, 3 to 30, 1 to 5, 1 to 10, 1 to 8, 1 to 6, 1, 2, 3, 4 or 5;
X¹ is O, S or N—R; and
R is as described above, or a pharmaceutical salt thereof.

The term “connector”, symbolized by [CON], is used to describe a chemical moiety which is optionally included in chimeric compositions according to the present invention which forms from the reaction product of an activated CARBM-linker with a PBM moiety (which also is preferably activated) or a CARBM moiety with an activated linker-PBM moiety as otherwise described herein. The connector group is the resulting moiety which forms from the facile condensation of two separate chemical fragments which contain reactive groups which can provide connector groups as otherwise described to produce chimeric compositions according to the present invention. It is noted that a connector may be distinguishable from a linker in that the connector is the result of a specific chemistry which is used to provide chimeric compounds according to the present invention wherein the reaction product of these groups results in an identifiable connector group which is distinguishable from the linker group as otherwise described herein. It is noted that there may be some overlap between the description of the connector group and the linker group, especially with respect to more common connector groups such as amide groups, oxygen (ether), sulfur (thioether) or amine linkages, urea or carbonate —OC(O)O— groups as otherwise described herein. It is further noted that a connector (or linker) may be connected to CARBM, a linker or PBM at positions which are represented as being linked to another group using the using the symbol

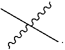

Where two or more such groups are present in a linker or connector, any of a CARBM, a linker or a PBM may be bonded to such a group.

Common connector groups which are used in the present invention include the following chemical groups:

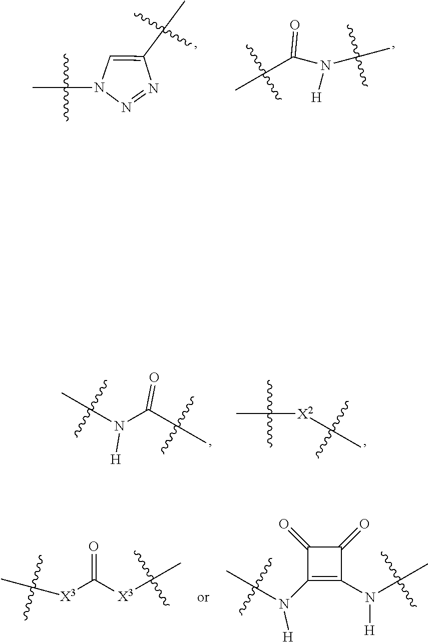

Where X² is O, S, NR⁴, S(O), S(O)₂, —S(O)₂O, —OS(O)₂, or OS(O)₂O;
X³ is O, S, NR⁴; and
R⁴ is H, a C₁-C₃ alkyl or alkanol group, or a —C(O)(C₁-C₃) group. A triazole group is often preferred.

The term “pharmaceutically acceptable salt” or “salt” is used throughout the specification to describe a salt form of one or more of the compositions herein which are presented to increase the solubility of the composition in saline for parenteral delivery or in the gastric juices of the patient's gastrointestinal tract in order to promote dissolution and the bioavailability of the composition. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts may be preferred as neutralization salts of carboxylic acids and free acid phosphate containing compositions according to the present invention. The term “salt” shall mean any salt consistent with the use of the compounds according to the present invention. In the case where the compounds are used in pharmaceutical indications, including the treatment of prostate cancer, including metastatic prostate cancer, the term “salt” shall mean a pharmaceutically acceptable salt, consistent with the use of the compounds as pharmaceutical agents.

The term “self-labeling polypeptide tag”, “self-labeling tag” “tag moiety” or halotag, snaptag and/or cliptag moiety is used to describe a tag moiety on bi-functional compounds according to the present invention which are used in preferred embodiments according to the present invention as a means to covalently link the tag moiety on the bi-functional molecule to the antigen binding region of the CAR. In this way, the bi-functional molecule becomes covalently linked to the CAR, reacted at one end through the tag moiety and bound to a cancer binding moiety (CBM or PBM) at the other end of the bi-functional moiety through a linker which can function to target CAR T cells to cancer cells, especially prostate cancer cells, including metastatic and recurrent prostate cancer cells. In certain embodiments of the present invention, the antigen binding region comprises a tag labeling enzyme (often mutated) which is expressed in the CAR polypeptide in the antigen binding region and is generally disposed on or near the cell surface of the cell which expresses the CAR polypeptide. The enzyme is reactive with a specific tag moiety on the bi-functional molecule which binds to the enzyme in order to covalently bind the bi-functional molecule to the CAR polypeptide. The tag moiety binds to the enzyme in the antigen binding region of the CAR polypeptide and is acted thereon by the enzyme to provide a covalent bond which binds the CAR polypeptide to the bi-functional molecule. The bi-functional molecule once bound, is capable of targeting cancer cells through the cancer binding moiety (CBM or PBM) as otherwise disclosed herein. Preferred tag moieties include, for example, halotag, snaptag or cliptag self-labeling tags. All of the tag enzymes for incorporation into vectors which express CAR polypeptides are readily available in commercially available expression vectors from Promega Corporation of Madison, Wisconsin (halotag) and New England BioLabs, Inc. of Ipswich, Massachussets, which vectors can accommodate the splicing of a gene for a protein of interest into the expression vector in order to produce the polypeptide comprising the protein of interest CAR which includes a self-labeling polypeptide tag enzyme as the antigen binding region of the CAR polypeptide.

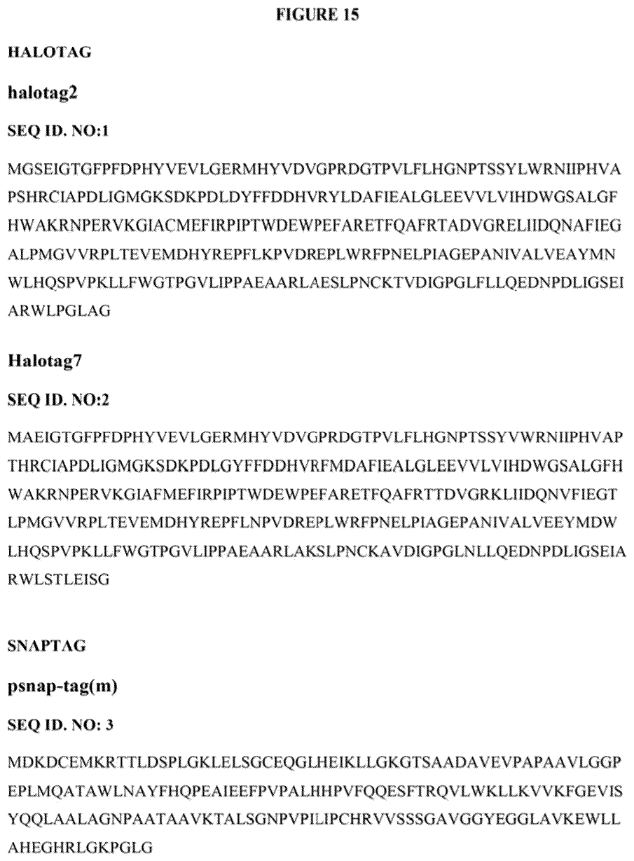

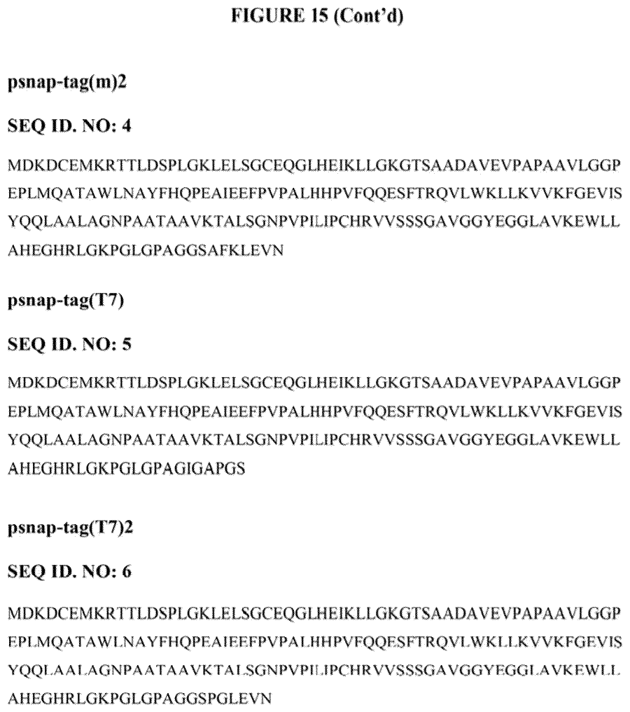

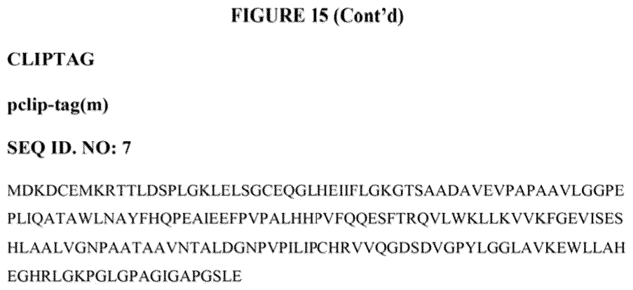

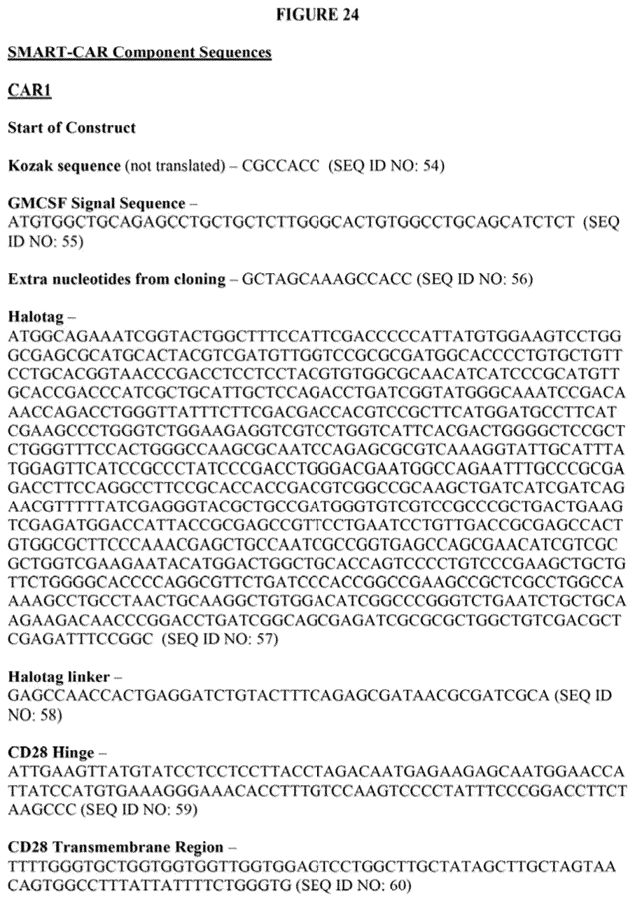

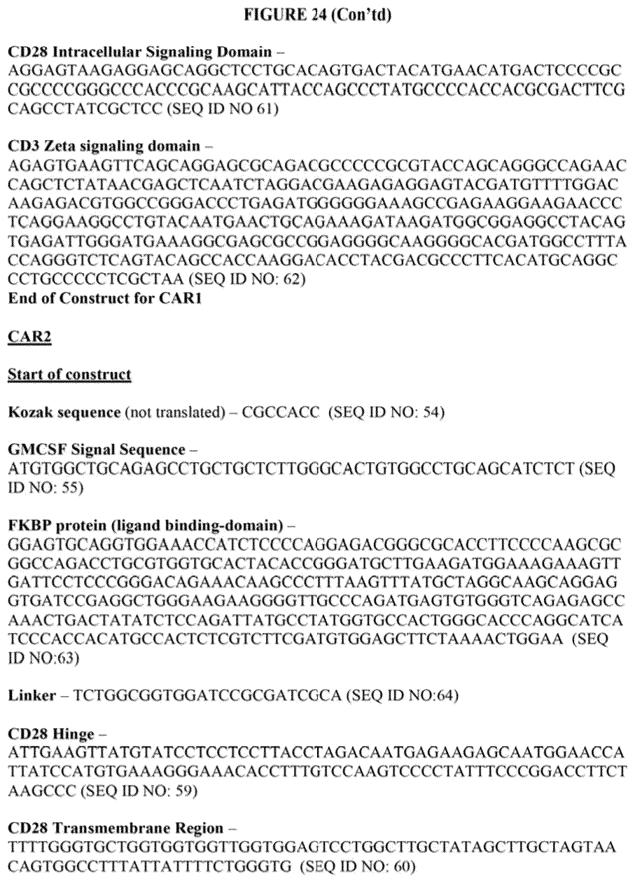

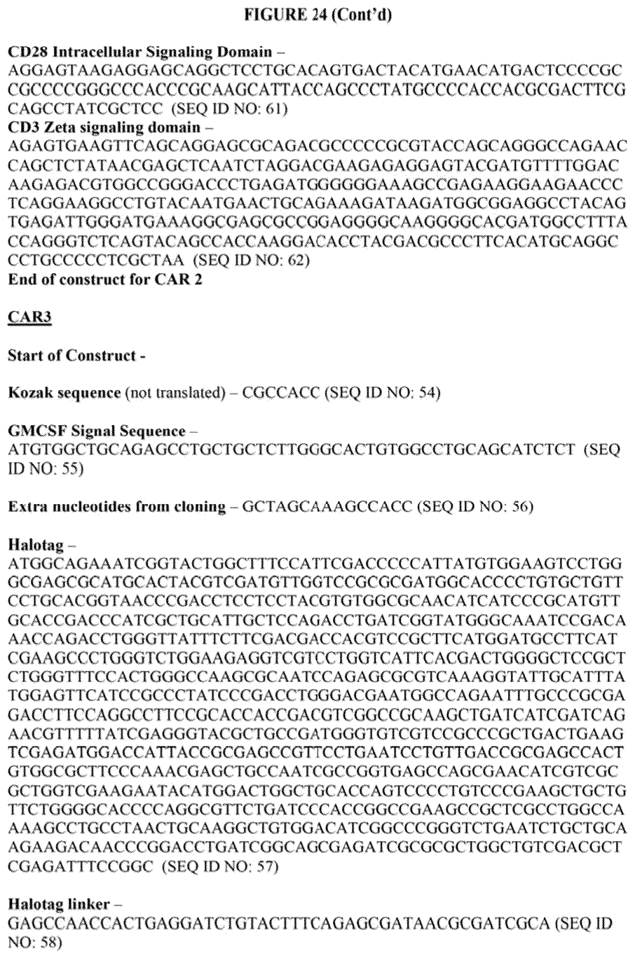

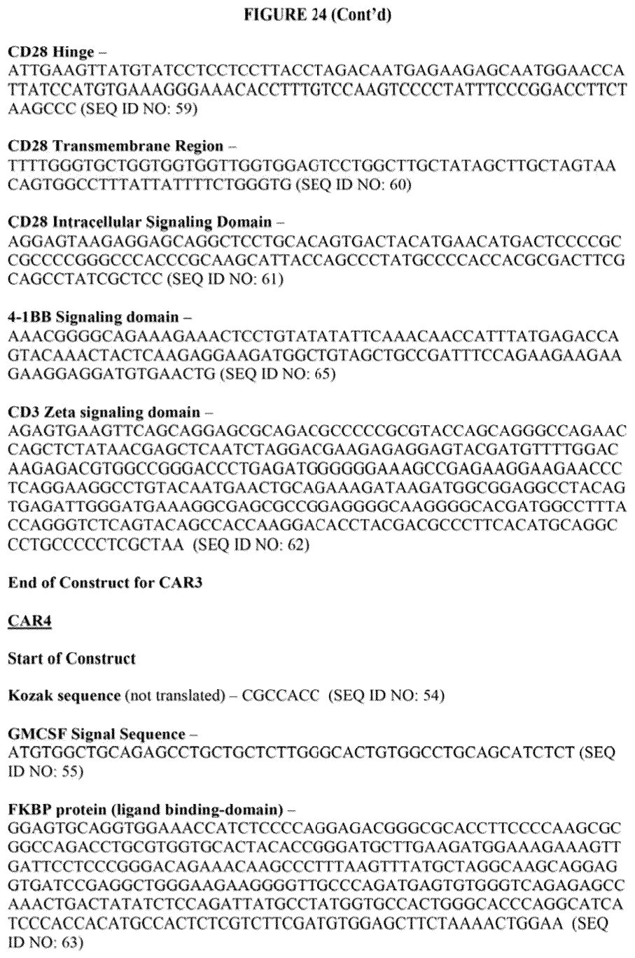

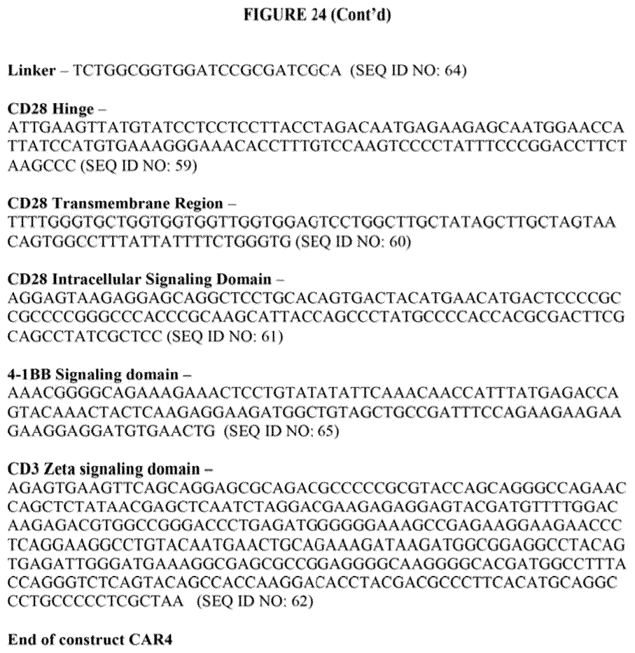

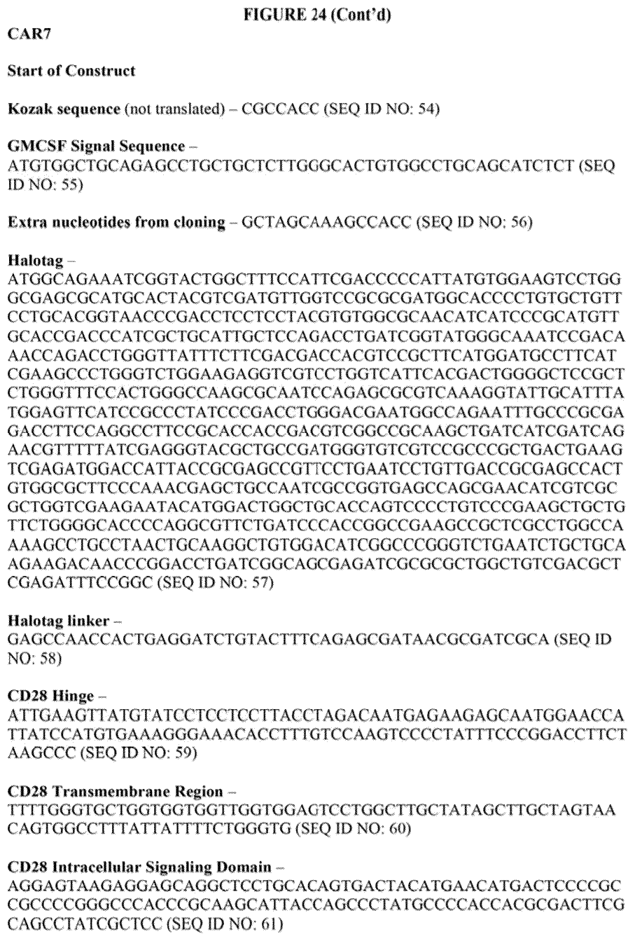

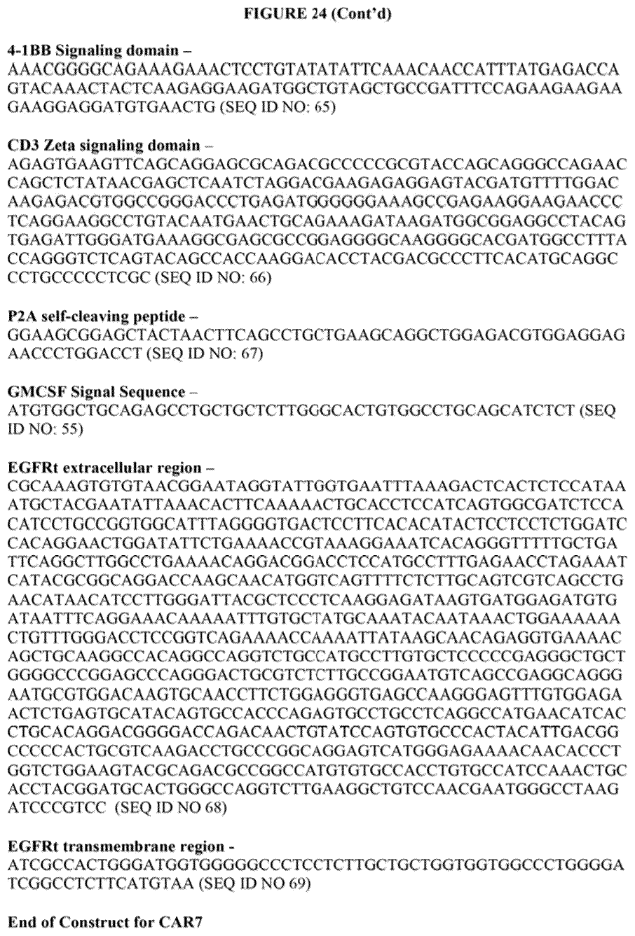

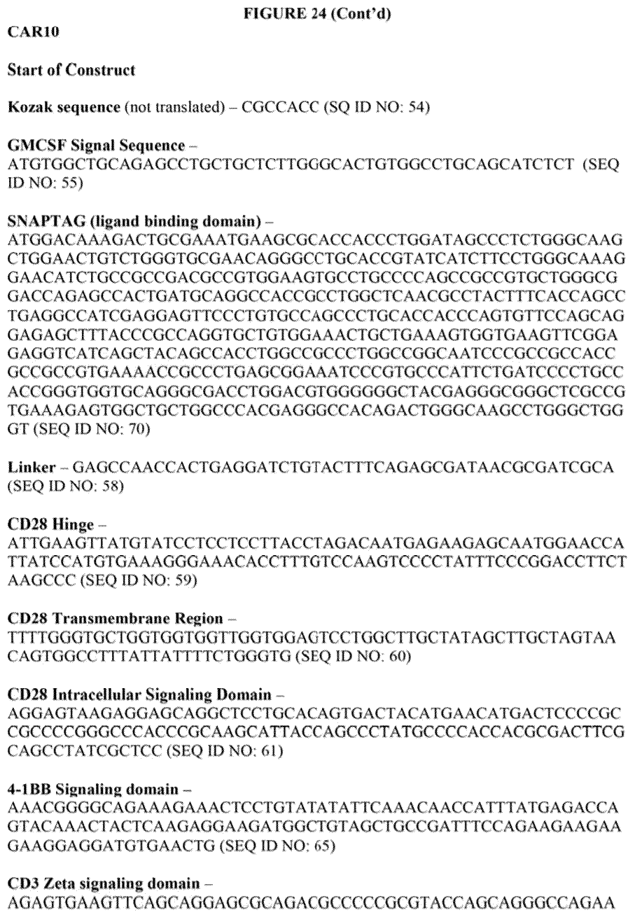

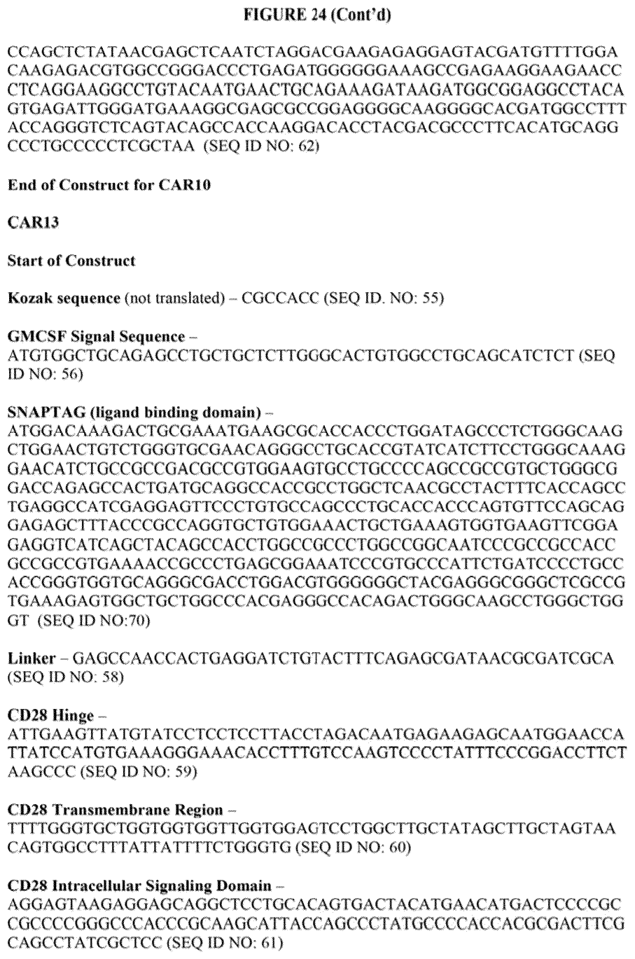

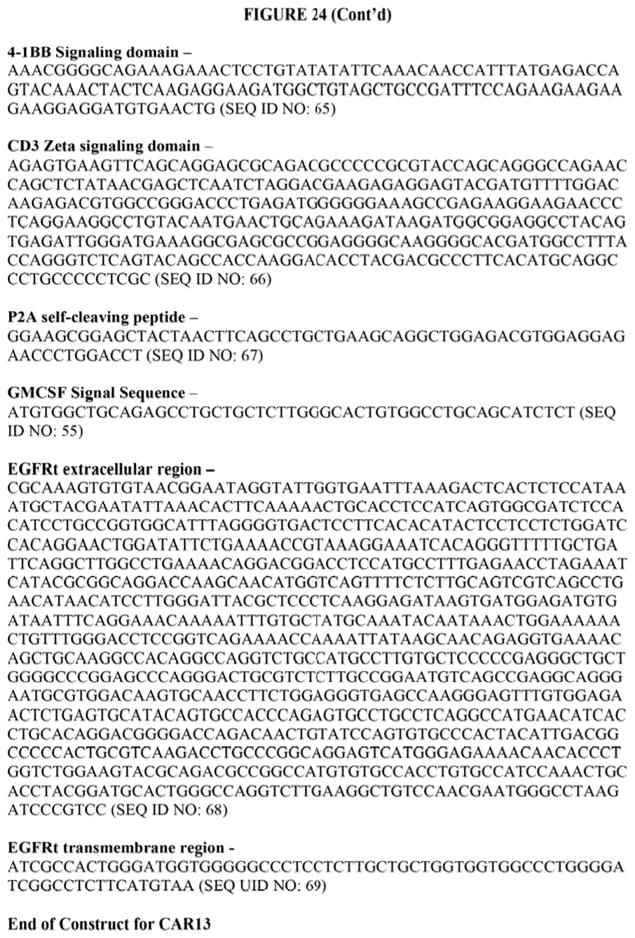

The halotag self-labeling polypeptide tag is based upon the halotag protein, a 34 kDa mutated bacterial hydrolase (haloalkane dehalogenase) which has been incorporated into expression vectors by Promega corporation, which are available commercially. For example, the halotag2 self-labeling tag (haloalkane dehalogenase) sequence SEQ ID NO: 1 (see FIG. 15) may be found at GenBank® Acc. #. AAV70825 and the expression vector at AY773970, among others. The halotag7 polypeptide is SEQ ID NO:2 (FIG. 15). A DNA sequence for incorporating the halotag into a CAR polypeptide is presented in FIG. 24 (SEQ ID NO: 57). The halotag polypeptide is reactive with haloalkanes and when expressed in CAR polypeptides according to the present invention, creates a covalent bond between the CAR polypeptide and a reactive haloalkane moiety onto which has been further linked a cancer binding moiety (CBM or PBM). Although a number of haloalkane groups may be used as the reactive linker in the halotag system as disclosed herein in order to create a covalent bond between the CAR polypeptide and the bi-functional molecule, the preferred reactive linker is or contains a chloroalkane, especially a chlorohexane group according to the structure

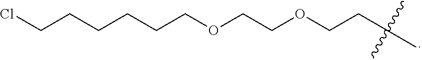 [SMILES: *CCOCCOCCCCCCCl]

The halogtag can readily accommodate C₃-C₈ haloalkane (preferably chloro) groups within this moiety. The halogtag is readily available in commercially available expression vectors from Promega Corporation of Madison, Wisconsin (halotag). These vectors can accommodate the splicing of a gene for the protein of interest (e.g., the CAR polypeptide according to the present invention into the expression vector in order to produce the CAR polypeptide which comprises the self-labeling polypeptide tag, expressed in numerous expression vectors well known in the art.

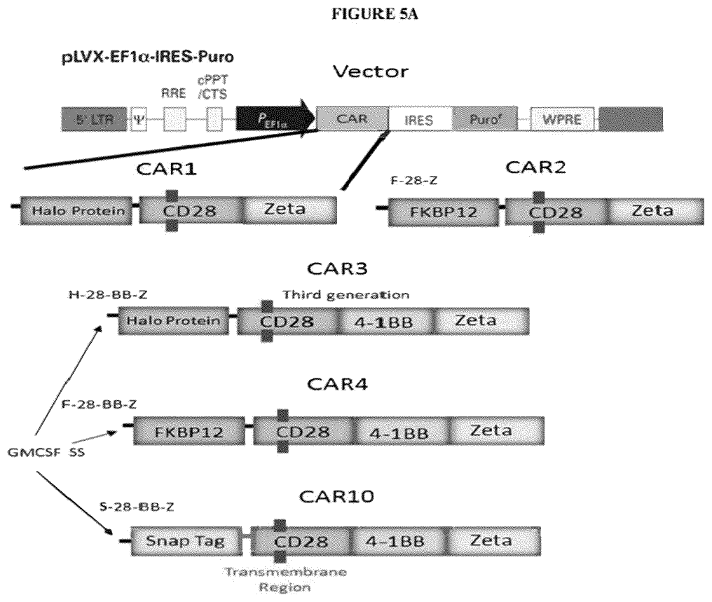

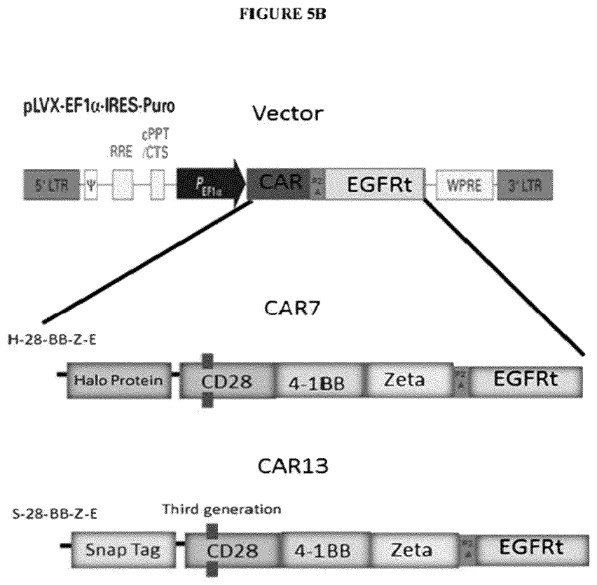

The snaptag self-labeling polypeptide tag is based upon a 20 kDa mutant of the DNA repair protein O⁶-alkylguanine-DNA alkyltransferase that reacts specifically and rapidly with O6-benzylguanine (BG) derivatives as otherwise described herein, leading to irreversible covalent labeling of the snaptag with the bi-functional molecule which contains the cancer binding moiety (CBM or PBM) through a sulfur group residing on the snaptag and the benzyl group of the benzylguanine synthetic probe (displacing guanine and binding to the benzyl group). The rate of the reaction of snaptag with BG derivatives is to a large extent independent of the nature of the synthetic probe attached to BG in the present bi-functional molecules and permits the labeling of snap fusion proteins with a wide variety of synthetic probes. Expression vectors for incorporating snaptag into numerous fusion proteins (e.g. psnap-tag(m), psnap-tag(m)2, psnap-tag(T7) and psnap-tag (T7)-2 Vector, among others) are available from New England Biolabs, Inc., USA. The polypeptide sequences for each of the snaptag polypeptides (snaptagm, snaptagm2, snaptagT7 and snaptagT7-2) are found in FIG. 15 as psnap-tag(m) (SEQ ID NO:3), psnap-tag(m)2 (SEQ ID NO:4), psnap-tag(T7) (SEQ ID NO:5) and psnap-tag (T7)-2 (SEQ ID NO:6). A DNA sequence for incorporating a snaptag into a CAR polypeptide is SEQ ID NO: 70) which was incorporated into CAR10 polypeptide of FIG. 5.

The cliptag self-labeling polypeptide tag is based upon a mutation of the snaptag DNA alkyltransferase enzyme, resulting in differential substrate specificity. In the case of cliptag protein, this protein reacts specifically with O2-benzylcytosine (BC) derivatives forming a covalent bond between a synthetic probe which is attached to O2-benzylcystosine and the cliptag through a sulfur group on the cliptag and the benzyl group on the benzylcytosine derivative. The SNAP- and CLIP-tag proteins can be covalently labeled with different synthetic tags in CAR expressing T cells as described herein to provide CAR T cells to which are conjugated bi-functional molecules which can specifically target cancer cells through the cancer binding moiety (CBM or PBM). Expression vectors for incorporating cliptag into numerous fusion proteins (e.g. clip-tag(m) vector are available from New England Biolabs, Inc., USA). The polypeptide sequence for the cliptag polypeptide (cliptagm) is found in FIG. 15 as pclip-tag(m) (SEQ ID NO:7).

The present invention provides chimeric antigen receptor (CAR) compositions, methods of making and using thereof.

A chimeric antigen receptor (CAR) polypeptide useful in the present invention includes an antigen recognition domain, a hinge region, a transmembrane domain, at least one co-stimulatory domain, and a signaling domain.

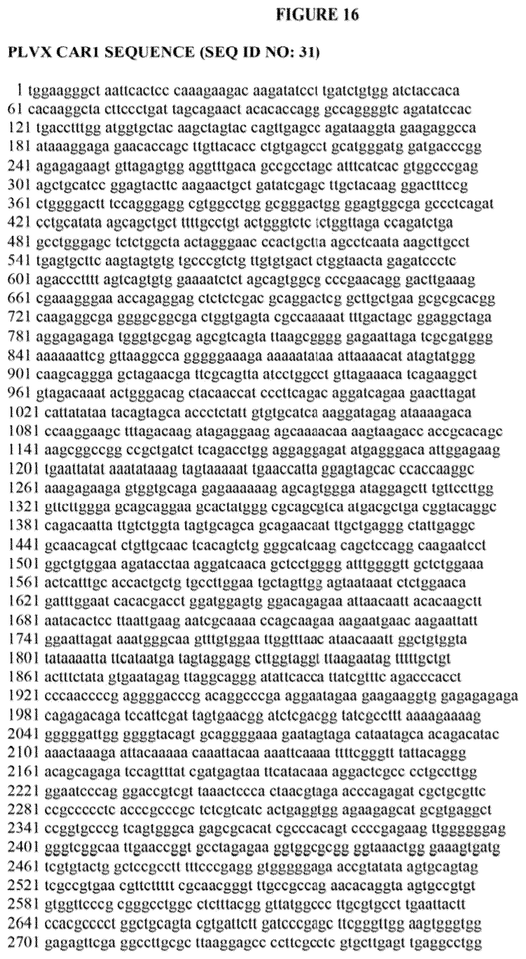

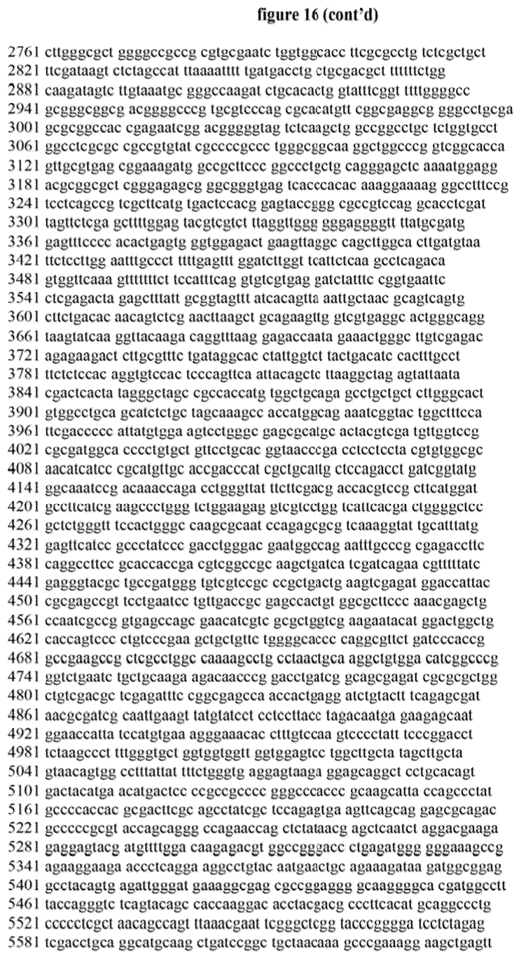

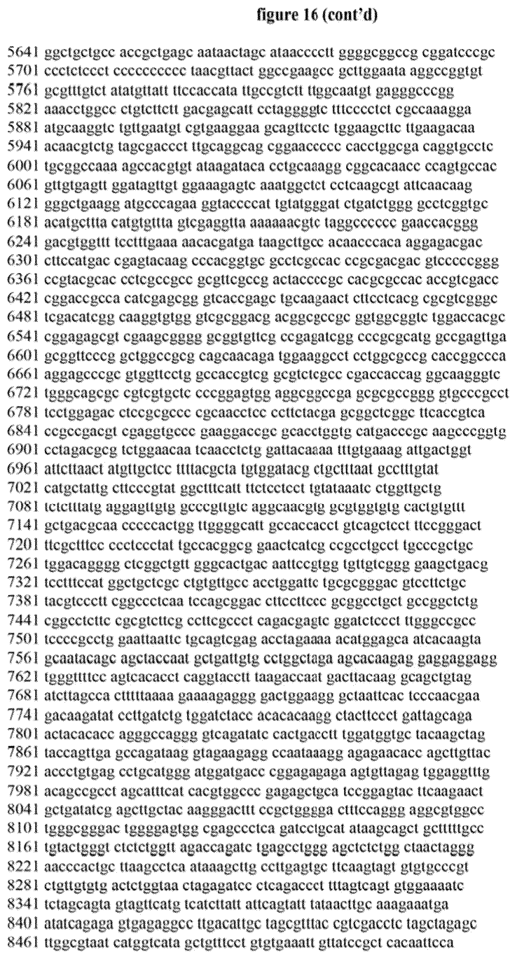

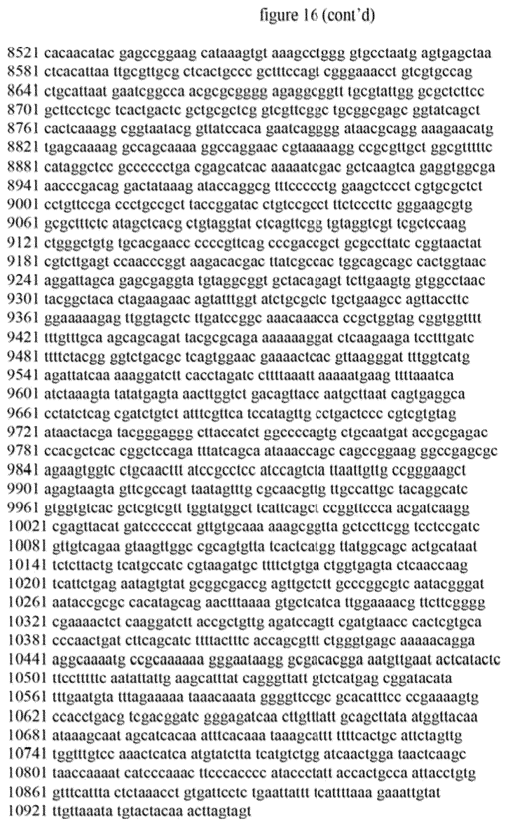

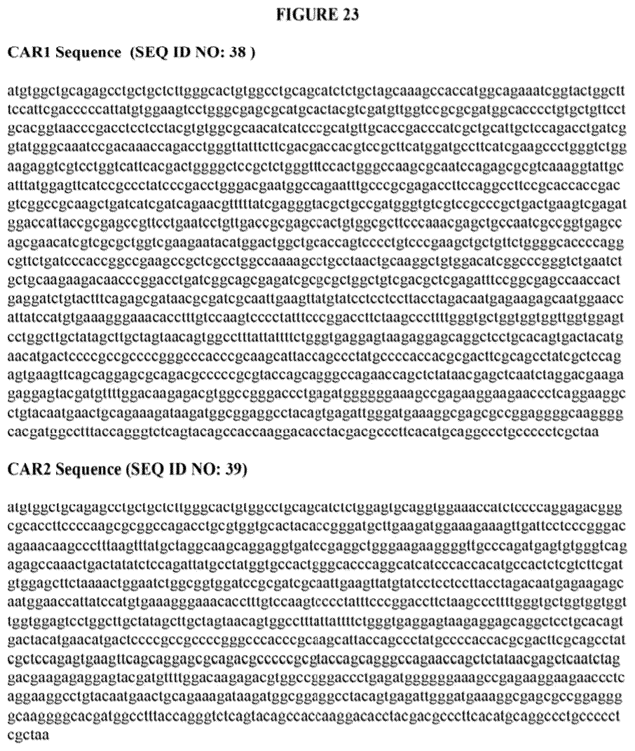

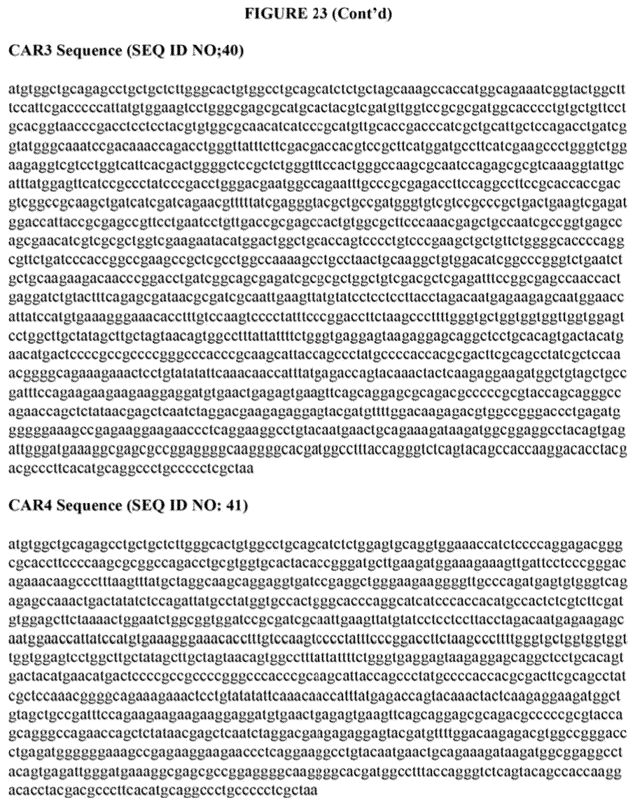

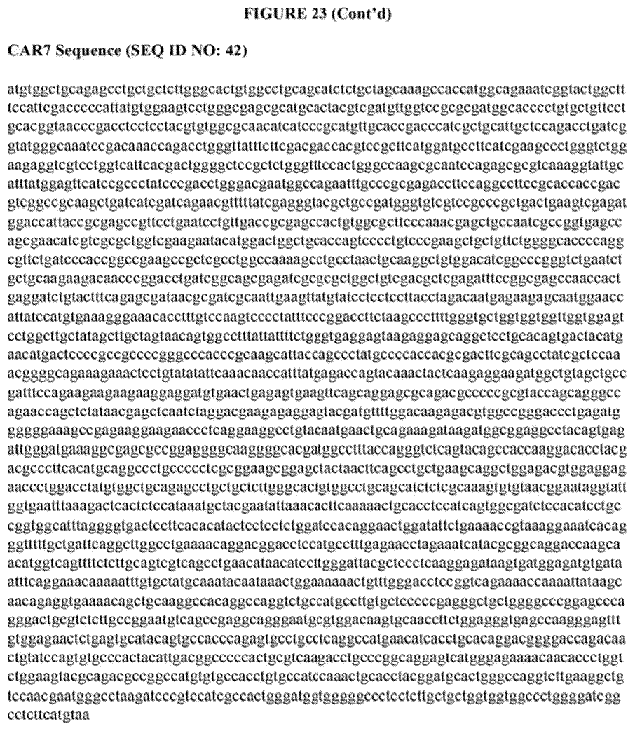

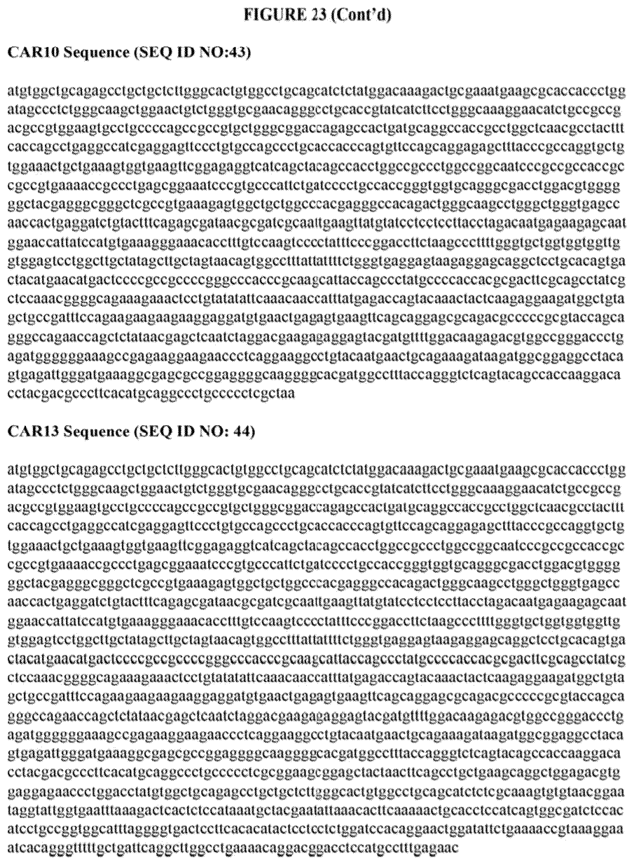

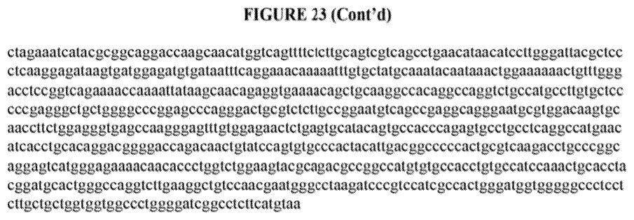

First-generation CARs include halotag protein or FRKP12 as an antigen binding domain, CD28 as a single transmembrane domain which includes a co-stimulatory domain and CD3z as an intracellular signaling domain, whereas third-generation CARs include at least one single additional co-stimulatory domain derived from various proteins. Examples of co-stimulatory domains include, but are not limited to, CD28, CD2, 4-1BB (CD137, also referred to as “4-1BB”), and OX-40 (CD124). Third generation CARs include two co-stimulatory domains, such as, but not limited to, CD28, 4-1BB, CD134 (OX-40), CD2, and/or CD137 (4-1BB). Preferably, CD28 and 4-1BB are the two co-stimulatory domains utilized in chimeric antigen receptors according to the present invention. A number of preferred CAR polypeptides are presented in FIG. 5 hereto. Their sequences are presented in FIG. 23 hereof. Vectors which have been prepared and cloned and are used to express the CAR polypeptides in transduced T cells are presented in FIGS. 16-22.

The terms “peptide,” “polypeptide,” and “protein” are used interchangeably, and refer to a compound having amino acid residues covalently linked by peptide bonds. A protein or peptide must contain at least two amino acids, and no limitation is placed on the maximum number of amino acids that can be included in a protein's or peptide's sequence. Polypeptides include any peptide or protein having two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, and fusion proteins, among others. The polypeptides include natural peptides, recombinant peptides, synthetic peptides, or a combination thereof.

A “signal peptide” includes a peptide sequence that directs the transport and localization of the peptide and any attached polypeptide within a cell, e.g. to a certain cell organelle (such as the endoplasmic reticulum) and/or the cell surface. As used herein, “signal peptide” and “leader sequence” are used interchangeably. The signal peptide is a peptide of any secreted or transmembrane protein that directs the transport of the polypeptide of the disclosure to the cell membrane and cell surface, and provides correct localization of the polypeptide of the present disclosure. In particular, the signal peptide used in the present invention directs the CAR polypeptide to the cellular membrane, wherein the extracellular portion of the polypeptide is displayed on the cell surface, the transmembrane portion spans the plasma membrane, and the active domain is in the cytoplasmic portion, or interior of the cell.

In one embodiment, the signal peptide is cleaved after passage through the endoplasmic reticulum (ER), i.e. is a cleavable signal peptide. In an embodiment, the signal peptide is human protein of type I, II, Ill, or IV. In an embodiment, the signal peptide includes an immunoglobulin heavy chain signal peptide. Preferred signal peptides are presented in FIG. 24 hereof. A preferred signal peptide is a GMCSF signal peptide encoded by the polynucleotide of SEQ ID NO: 55.

The “antigen recognition domain” includes a polypeptide that is selective for or targets an antigen, receptor, peptide ligand, or protein ligand of the target; or a polypeptide of the target.

In the present invention, the antigen recognition domain comprises a halotag protein, a snaptag protein, a cliptag protein or a member of the immunophilin (FKBP) family of proteins (FK506 binding proteins), preferably a human protein and is preferably selected from the group consisting of FKBP3 (UniProtKB/Swiss-Prot Accession Number Q00688.1, same as FKBP25), FKPB5 (Q13451.2), FKBP9 (O95302.2), FKBP12 (P62942.2), FKBP12.6 (P68106.2), FKBP13 (P26885.2), FKBP15 (Q5T1M5.2), FKBP22 (Q9NWM8), FKBP36 (O75344.1), FKBP38 (Q14318.2), FKBP51 (Q02790.3), FKBP65 (Q9FJL3.1) and FKBP133 (Q6P9Q6.2) or an isoform or fragment thereof which binds to a FKBP binding moiety as otherwise described herein.

It is understood that the antigen recognition domain may include some variability within its sequence and still be selective for the targets disclosed herein. Therefore, it is contemplated that the polypeptide of the antigen recognition domain may be at least 95%, at least 90%, at least 80%, or at least 70% identical to the antigen recognition domain polypeptides disclosed herein and still be selective for the targets described herein and be within the scope of the disclosure of the present invention.

The target includes moieties which bind and are acted on by halotag protein (C₃-C₈ haloalkanes, especially chloroalkanes), snaptag protein (O6-benzylguanine) and cliptag protein O2-benzylcytosine) as described herein.

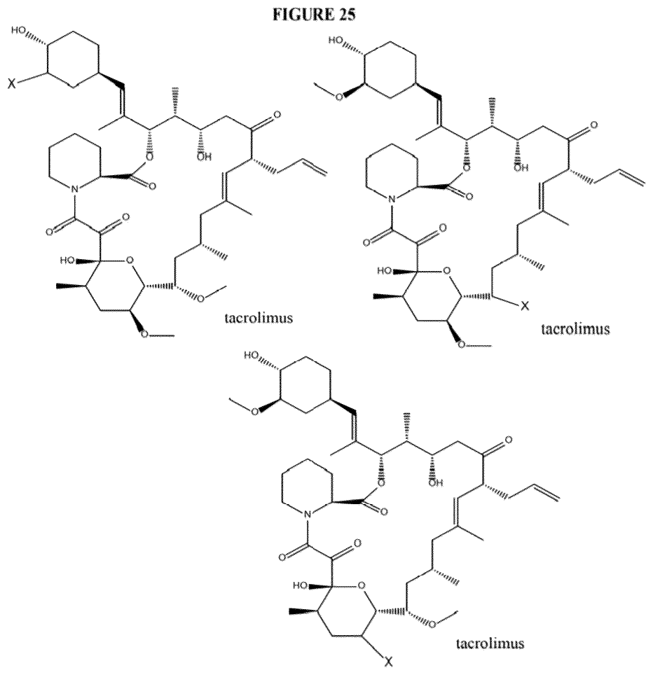

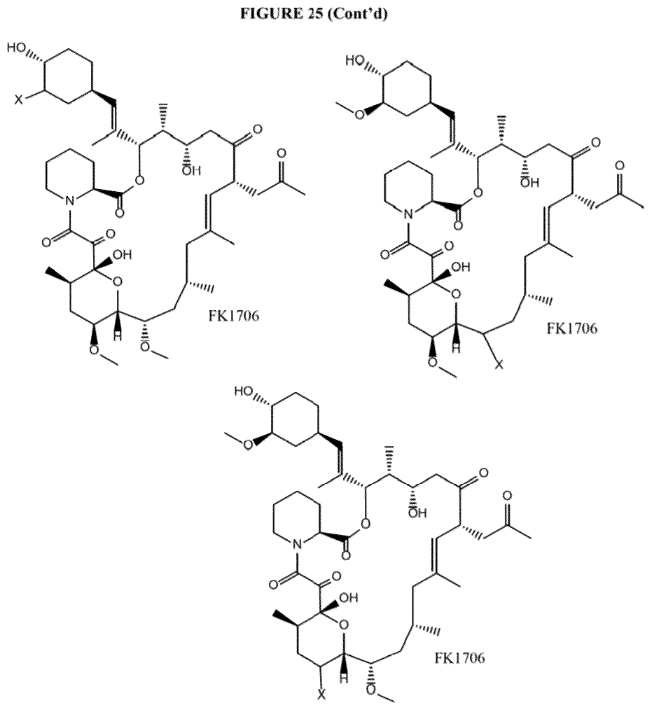

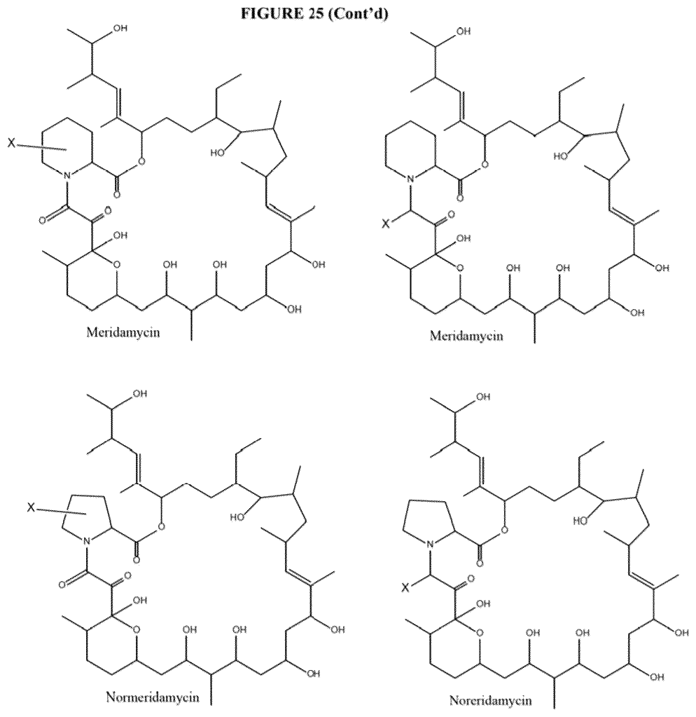

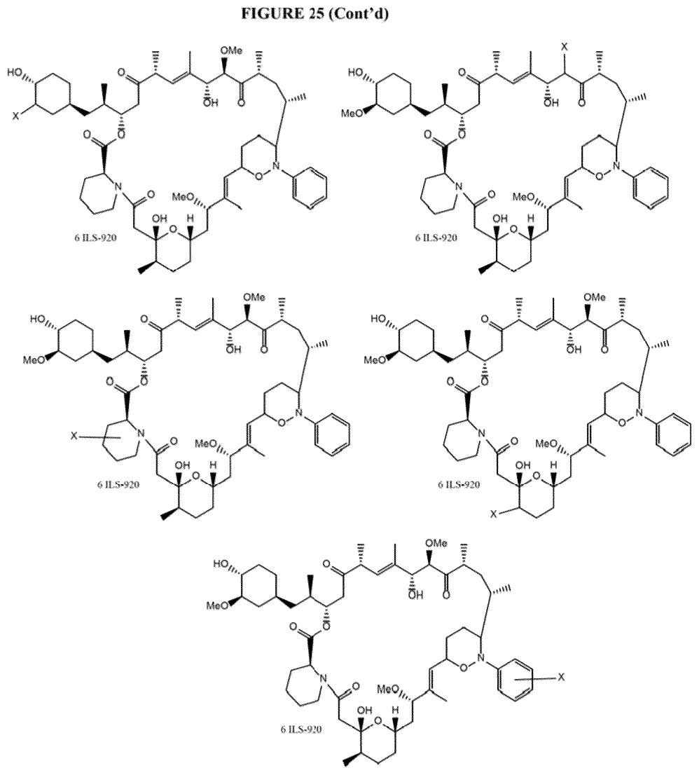

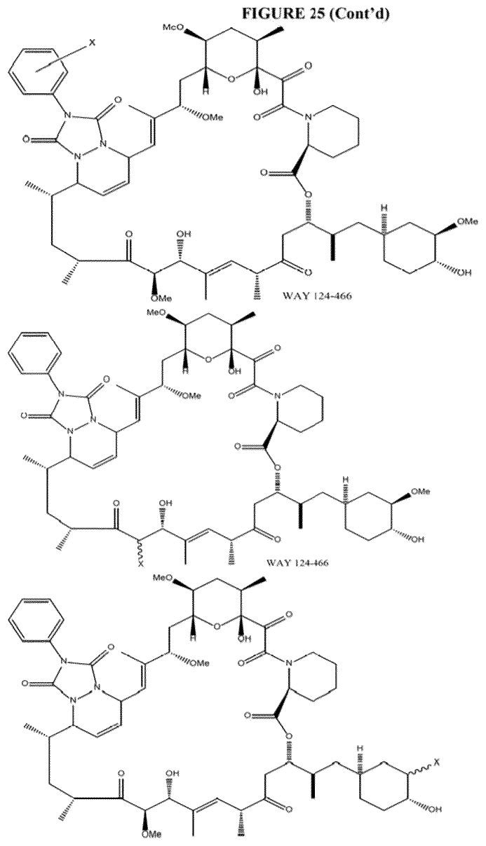

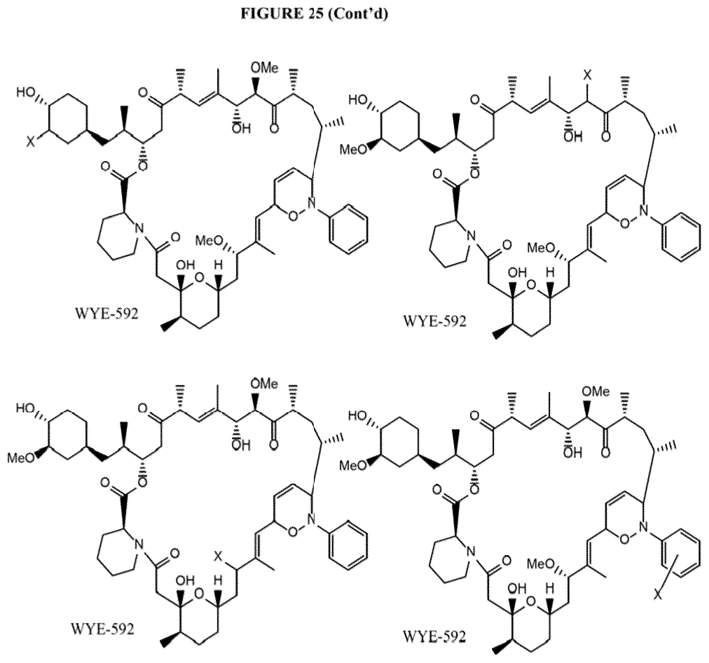

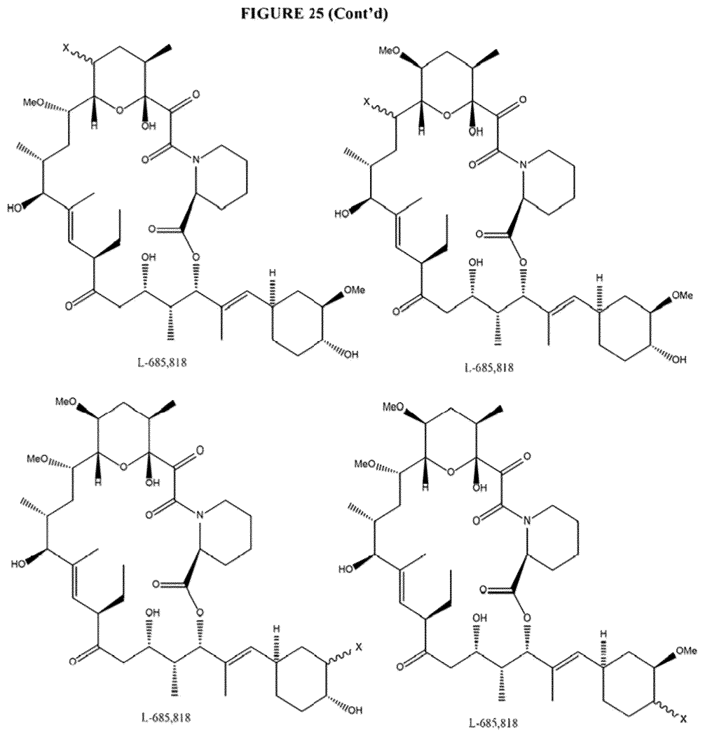

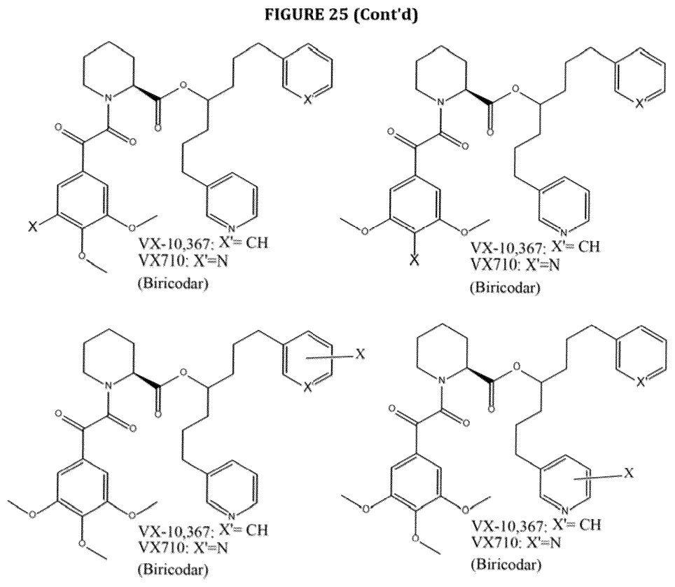

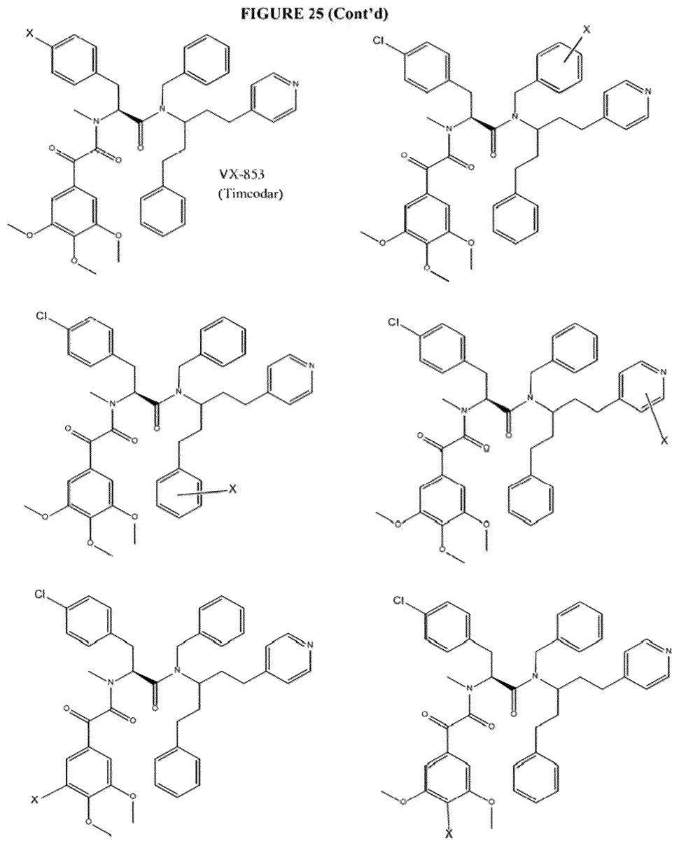

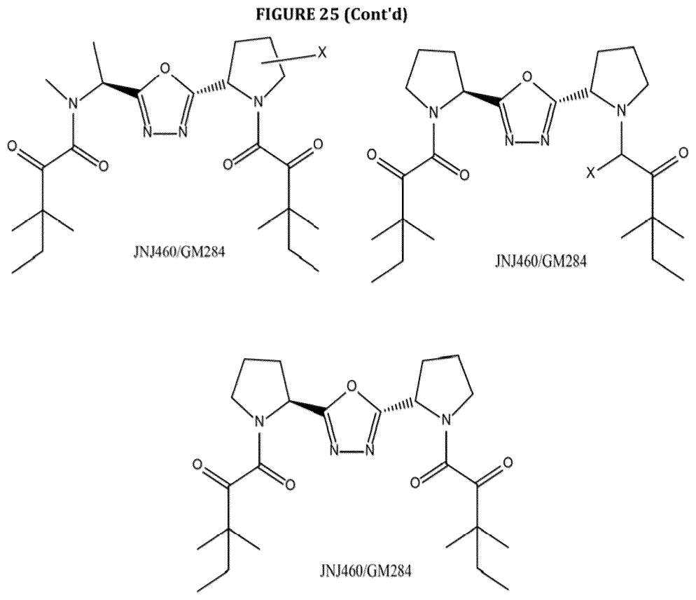

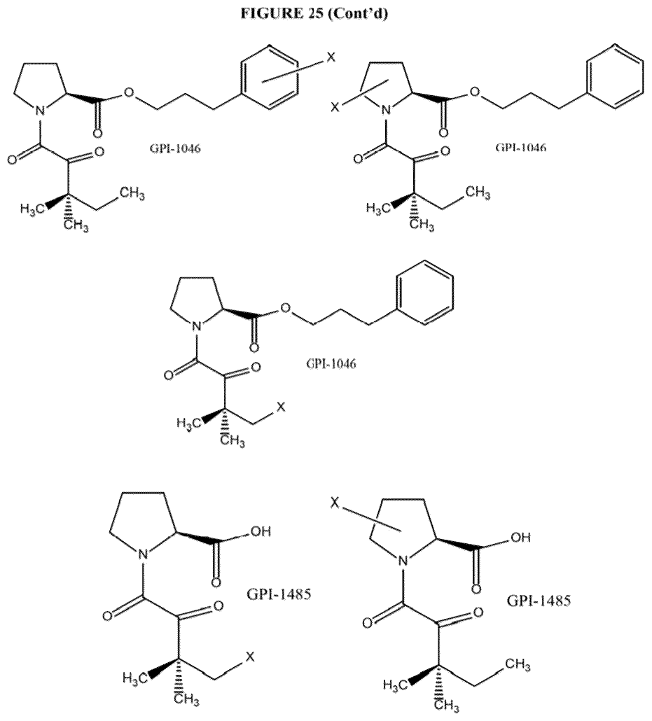

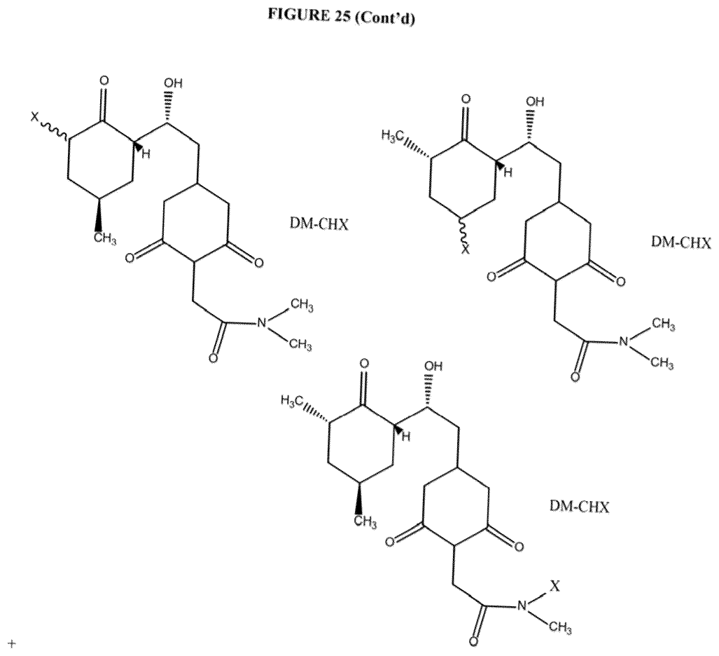

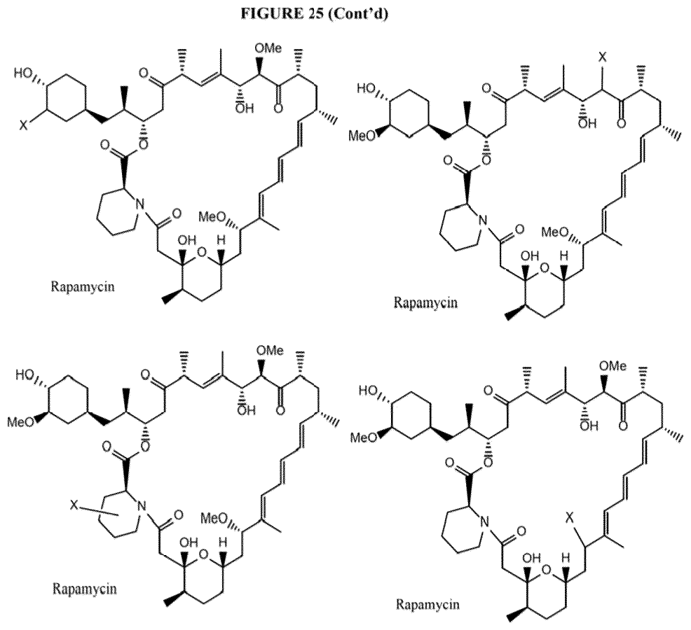

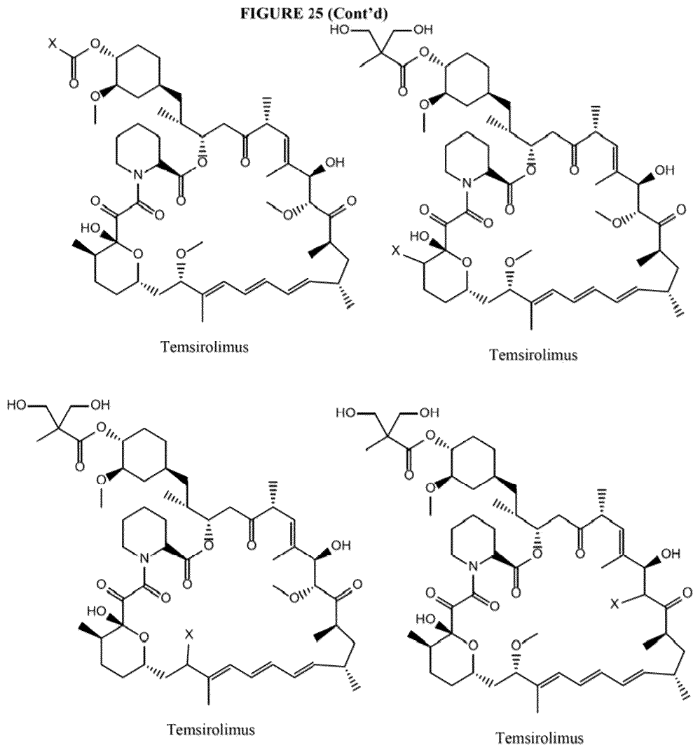

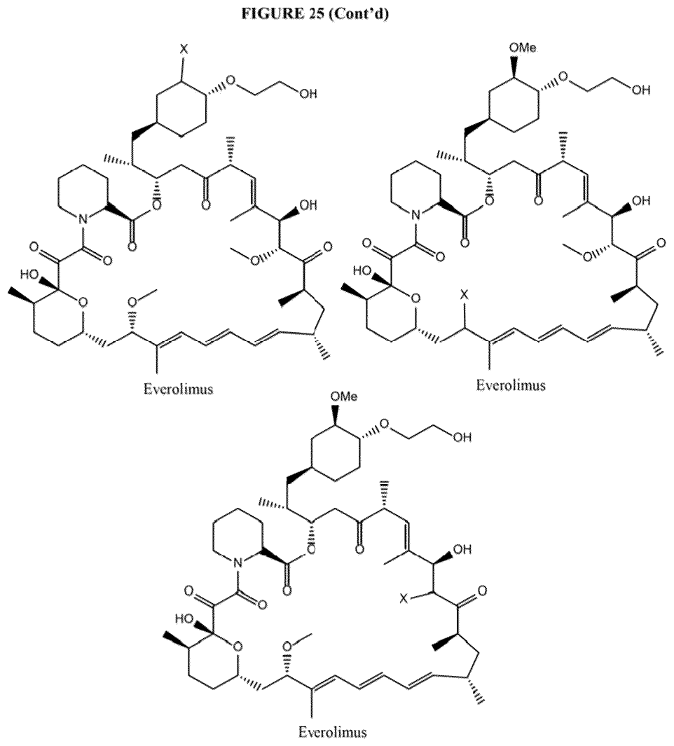

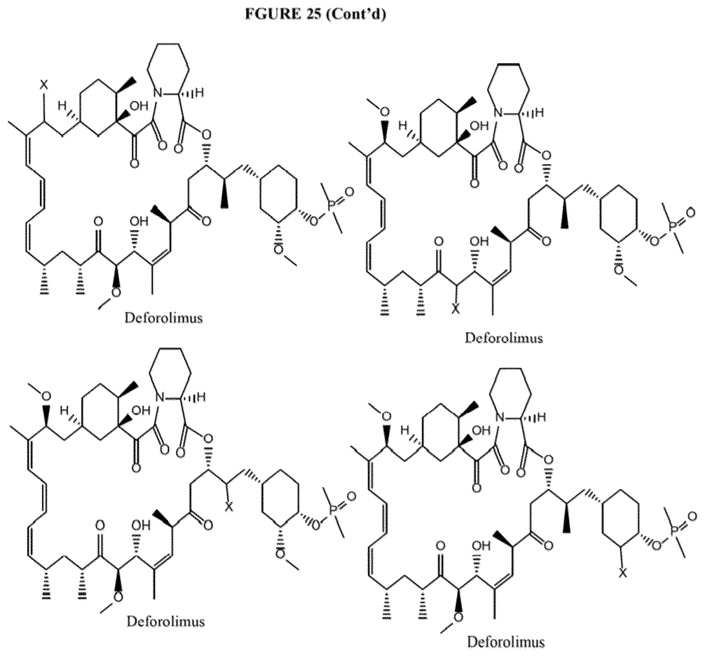

In another embodiment, the target includes any moiety which binds to a member of the immunophilin (FKBP) family of proteins (FK506 binding proteins) and includes moieties which bind to the FKBP (FKBP binding moiety) and which is selected from the group consisting of FK506 (tacrolimus), a FK506 derivative or a rapalog. FK506 derivatives include but are not limited to FK1706, meridamycin, normeridamycin, ILS920, Way-124466, Wye-592, L685-818, VX-10,367, VX-710 (Biricodar), VX-853 (Timcodar), JNJ460/GM284, GP11046, GP11485 and DM-CHX; useful rapalogs include but are not limited to rapamycin (sirolimus), temsirolimus (CCI 779), everolimus (RAD001) and ridaforolimus/deforolimus (AP-23573). These moieties and their attachment points for inclusion in bi-functional molecules according to the present invention are presented in FIG. 25 hereof.

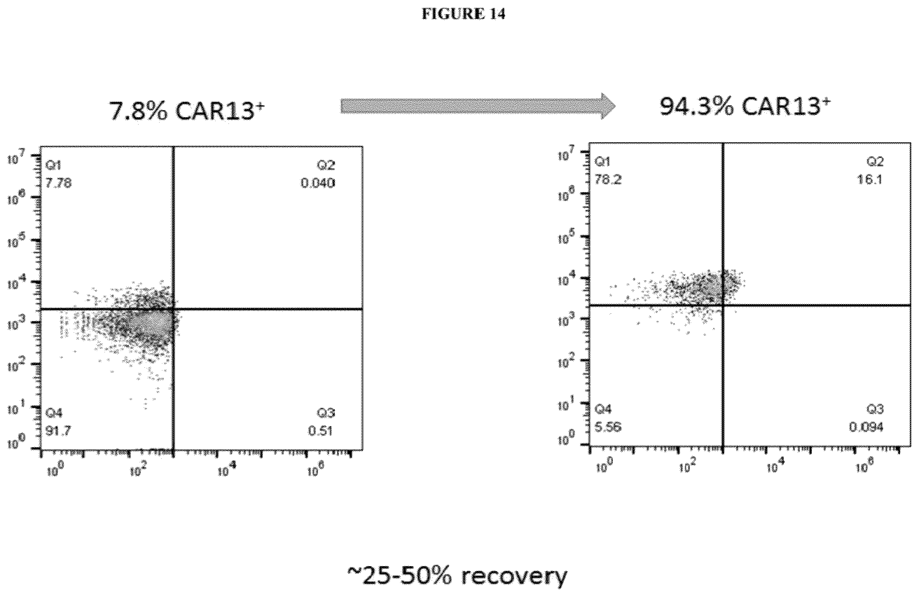

In one embodiment, the halotag antigen recognition domain includes SEQ ID NO: 1 of halotag 2 or SEQ ID NO: 2 halotag 7 of FIG. 14, or the DNA sequence SEQ ID NO: 57 of FIG. 24.

In one embodiment, the snaptap antigen recognition domain includes p-snaptag(m) SEQ ID NO: 3, p-snaptag(m)2 SEQ ID NO: 4, p-snaptag(T7) SEQ ID NO: 5 and p-snaptag(T7)2 SEQ ID NO: 6 of FIG. 14 or the DNA sequence SEQ ID NO: 70 of FIG. 24.

In one embodiment, the cliptag antigen recognition domain includes p-cliptag(m) SEQ ID NO: 7 of FIG. 14.

The “hinge region” is a sequence positioned between for example, including, but not limited to, the antigen binding domain and at least one co-stimulatory domain and a signaling domain. The hinge sequence may be obtained including, for example, from any suitable sequence from any genus, including human or a part thereof. Such hinge regions are known in the art. In an embodiment, the hinge region includes the hinge region of a human protein including CD28, 4-1BB, OX40, CD3-zeta, CD-8 alpha, T cell receptor α or β chain, a CD3 zeta chain, CD28, CD3epsilon, CD45, CD4, CD5, CD8, CD8a, CD9, CD16, CD22, CD33, CD37, CD64, CD80, CD86, CD134, CD137, ICOS, CD154, functional derivatives thereof, and combinations thereof. Preferred hinge regions for use in CAR polypeptides are presented in FIG. 24 hereof.

In one embodiment the hinge region includes the human CD28 hinge region. In some embodiments, the hinge region includes the human CD28 hinge region, the human 4-1BB hinge region or the human CD3-zeta human hinge region.

The transmembrane domain includes a hydrophobic polypeptide that spans the cellular membrane. In particular, the transmembrane domain spans from one side of a cell membrane (extracellular) through to the other side of the cell membrane (intracellular or cytoplasmic). The transmembrane domain may be in the form of an alpha helix or a beta barrel, or combinations thereof. The transmembrane domain may include a polytopic protein, which has many transmembrane segments, each alpha-helical, beta sheets, or combinations thereof.

In an embodiment, the transmembrane domain that is naturally associated with one of the domains in the CAR is used. In another embodiment, the transmembrane domain is selected or modified by amino acid substitution to avoid binding of such domains to the transmembrane domains of the same or different surface membrane proteins to minimize interactions with other members of the receptor complex.

For example, a transmembrane domain includes a transmembrane domain of CD28 (which is preferred), a T-cell receptor α or β chain, a CD3 zeta chain, a CD3-Epsilon, CD45, CD4, CD5, CD7, CD8, CD9, CD16, CD22, CD33, CD37, CD64, CD80, CD86, CD68, CD134, CD137, ICOS, CD41, CD154, functional derivatives thereof, and combinations thereof. In preferred embodiments a transmembrane domain of human CD28 or CD8 is used, more preferably human CD28. A DNA sequence for the cD28 transmembrane domain is presented as SEQ ID NO: 60 of FIG. 24. These transmembrane domains are well known in the art.

In one embodiment, the transmembrane domain may be artificially designed so that more than 25%, more than 50% or more than 75% of the amino acid residues of the domain are hydrophobic residues such as leucine and valine. In an embodiment, a triplet of phenylalanine, tryptophan and valine is found at each end of the synthetic transmembrane domain.

The signaling domain and co-stimulatory domain include polypeptides that provide activation of an immune cell to stimulate or activate at least some aspect of the immune cell signaling pathway. In an embodiment, the signaling domain includes the polypeptide of a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DNAX-activating protein 10 (DAP10), DNAX-activating protein 12 (DAP12), active fragments thereof, functional derivatives thereof, and combinations thereof. Such signaling domains are known in the art. Sequences for preferred components for incorporation into CAR polypeptides according to the present invention are presented in FIG. 24 hereof.

In an embodiment, the CAR polypeptide further includes one or more co-stimulatory domains. In an embodiment, the co-stimulatory domain is a functional signaling domain from a protein including one or more of 4-1BB/TNFRSF9/CD137, CD28, IL-15 receptor alpha; IL-15 receptor alpha cytoplasmic domain; B7-1/CD80; B7-2/CD86; CTLA-4; B7-H1/PD-L1; ICOS; B7-H2; PD-1; B7-H3; PD-L2; B7-H4; PDCD6; BTLA; CD40 Ligand/TNFSF5; 4-1BB Ligand/TNFSF9; GITR/TNFRSF18; BAFF/BLyS/TNFSF13B; GITR Ligand/TNFSF18; BAFF R/TNFRSF13C; HVEM/TNFRSF14; CD27/TNFRSF7; LIGHT/TNFSF14; CD27 Ligand/TNFSF7; OX40/TNFRSF4; CD30/TNFRSF8; OX40 Ligand/TNFSF4; CD30 Ligand/TNFSF8; TACI/TNFRSF13B; CD40/TNFRSF5; 2B4/CD244/SLAMF4; CD84/SLAMF5; BLAME/SLAMF8; CD229/SLAMF3; CD2, CD27, CRACC/SLAMF7; CD2F-10/SLAMF9; NTB-A/SLAMF6; CD48/SLAMF2; SLAM/CD150; CD58/LFA-3; Ikaros; CD53; Integrin alpha 4/CD49d; CD82/Kai-1; Integrin alpha 4 beta 1; CD90/Thy1; Integrin alpha 4 beta 7/LPAM-1; CD96; LAG-3; CD160; LMIR1/CD300A; CRTAM; TCL1A; DAP12; TIM-1/KIM-1/HAVCR; Dectin-1/CLEC7A; TIM-4; DPPIV/CD26; TSLP; EphB6; TSLP R; and HLA-DR, OX40; CD30; CD40; PD-1; CD7; CD258; Natural killer Group 2 member C (NKG2C); Natural killer Group 2 member D (NKG2D), B7-H3; a ligand that binds to at least one of CD83, ICAM-1, LFA-1 (CD1 la/CD18), ICOS, and 4-1BB (CD137); CDS; ICAM-1; LFA-1 (CD1a/CD18); CD40; CD27; CD7; B7-H3; NKG2C; PD-1; ICOS; active fragments thereof; functional derivatives thereof; and combinations thereof.

The at least one co-stimulatory domain and signaling domain may be collectively referred to as the intracellular domain. As used herein, the hinge region and the antigen recognition domain may be collectively referred to as the extracellular domain.

The present invention is also directed to a polynucleotide which encodes the chimeric antigen receptor polypeptide described herein. DNA sequences which encode for CAR polypeptides depicted in FIG. 5 hereof are presented in FIG. 23. The term “polynucleotide” as used herein is defined as a chain of nucleotides. Polynucleotide includes DNA and RNA. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction (PCR), and the like, and by synthetic means. The polynucleotide encoding the CAR is easily prepared from an amino acid sequence of the specified CAR by any conventional method. A base sequence encoding an amino acid sequence can be obtained from the aforementioned NCBI RefSeq IDs or accession numbers of GenBenk for an amino acid sequence of each domain, and the nucleic acid of the present disclosure can be prepared using a standard molecular biological and/or chemical procedure. For example, based on the base sequence, a polynucleotide can be synthesized, and the polynucleotide of the present disclosure can be prepared by combining DNA fragments which are obtained from a cDNA library using a polymerase chain reaction (PCR).

The polynucleotide described above is preferably cloned into a vector. A “vector” is a composition of matter which includes an isolated polynucleotide and which can be used to deliver the isolated polynucleotide to the interior of a cell. Numerous vectors are known in the art including, but not limited to, linear polynucleotides, polynucleotides associated with ionic or amphiphilic compounds, plasmids, phagemid, cosmid, and viruses. Viruses include phages, phage derivatives. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like. In one embodiment, vectors include cloning vectors, expression vectors, replication vectors, probe generation vectors, integration vectors, and sequencing vectors.

In preferred embodiments, the vector for the polynucleotide encoding the CAR is a viral vector. In an embodiment, the viral vector is a lentiviral vector, adenoviral vector r a retroviral vector, often a lentiviral vector. Preferred representative DNA sequences for the entire vector for each of the CAR polypeptides which are presented in FIG. 5 hereof are set forth in FIGS. 16-22. In an embodiment, an engineered cell is virally transduced for expression of the polynucleotide sequence.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the patient either in vivo or ex vivo. A number of retroviral systems are known in the art. In some embodiments, adenovirus vectors are used. A number of adenovirus vectors are known in the art. In one embodiment, lentivirus vectors are used.

Viral vector technology is well known in the art and is described, for example, in Sambrook et al, (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector for use in the present invention contains an origin of replication functional in at least one organism, a promoter sequence, convenient and unique restriction endonuclease sites in order to introduce peptides components, and one or more selectable markers, (e.g., WO 01/96584; WO 01/29058; and U.S. Pat. No. 6,326,193).

Lentiviral vectors, preferred vectors for use in the present invention have been well known for their capability of transferring genes into human T cells with high efficiency but expression of the vector-encoded genes is dependent on the internal promoter that drives their expression. A strong promoter is particularly important for the third or fourth generation of CARs that bear additional co-stimulatory domains or genes encoding proliferative cytokines as increased CAR body size does not guarantee equal levels of expression. There are a wide range of promoters with different strength and cell-type specificity. Gene therapies using CAR T cells rely on the ability of T cells to express adequate CAR body and maintain expression over a long period of time. In the present invention, the CMV promoter and most often the EF-1α promoter are preferably used. The CMV promoter and the EF-1α promoter have been commonly selected for the CAR expression.

The present invention provides an expression vector containing a strong promoter for high level gene expression in T cells or NK cells. In further embodiment, the present disclosure provides a strong promoter useful for high level expression of CARs in T cells or NK cells. In certain embodiments, the SFFV promoter is used, which is selectively introduced in an expression vector to obtain high levels of expression and maintain expression over a long period of time in T cells or NK cells. Certain expressed genes prefer CARs, T cell co-stimulatory factors and cytokines used for immunotherapy.

In the present invention, a preferred promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1 a (EF-1 a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the present invention is not limited to the use of constitutive promoters, and inducible promoters are also contemplated as part of the vector constructs of the present invention. In the present invention, the use of an inducible promoter provides a molecular switch capable of turning on expression of the polynucleotide sequence, which is operatively linked when such expression is desired, or turning off the expression when expression is not desired. Examples of inducible promoters include, but are not limited to a metalothionine promoter, a glucocorticoid promoter, a progesterone promoter, and a tetracycline promoter.

Expression of chimeric antigen receptor polynucleotide may be achieved using, for example, expression vectors including, but not limited to, at least one of a SFFV (spleen-focus forming virus) or human elongation factor 11α (EF) promoter, CAG (chicken beta-actin promoter with CMV enhancer) promoter human elongation factor 1α (EF) promoter. Examples of less-strong/lower-expressing promoters utilized may include, but are not limited to, the simian virus 40 (SV40) early promoter, cytomegalovirus (CMV) immediate-early promoter, Ubiquitin C(UBC) promoter, and the phosphoglycerate kinase 1 (PGK) promoter, or a part thereof. Inducible expression of chimeric antigen receptor may be achieved using, for example, a tetracycline responsive promoter, including, but not limited to, TRE3GV (Tet-response element, including all generations and preferably, the 3rd generation), inducible promoter (Clontech Laboratories, Mountain View, Calif.) or a part or a combination thereof.

In certain embodiments, the promoter is an SFFV promoter or a derivative thereof. The use of such a promoter often provides stronger expression and greater persistence in the transduced cells in accordance with the present disclosure.

The term “expression vector” refers to a vector including a recombinant polynucleotide comprising expression control sequences operatively linked to a nucleotide sequence to be expressed. An expression vector useful in the present invention includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. The expression vector may be a bicistronic or multicistronic expression vector. Bicistronic or multicistronic expression vectors may include (1) multiple promoters fused to each of the open reading frames; (2) insertion of splicing signals between genes; fusion of genes whose expressions are driven by a single promoter; (3) insertion of proteolytic cleavage sites between genes (self-cleavage peptide); and (iv) insertion of internal ribosomal entry sites (IRESs) between genes. In preferred aspects of the present invention, the expression vector is a lentiviral vector.

In one embodiment, the present invention is directed to an engineered cell having at least one chimeric antigen receptor polypeptide or polynucleotide.

The term “engineered cell” means any cell of any organism that is modified, transformed, or manipulated by addition or modification of a gene, a DNA or RNA sequence, or protein or polypeptide. Isolated cells, host cells, and genetically engineered cells of the present disclosure include isolated immune cells, especially including NK cells and T cells that contain the DNA or RNA sequences encoding a chimeric antigen receptor or chimeric antigen receptor complex and express the chimeric receptor on the cell surface. Isolated host cells and engineered cells may be used, for example, for the treat of cancer, especially prostate cancer or metastatic prostate cancer.

In the present invention, the engineered cell includes immunoregulatory cells. Immunoregulatory cells include T-cells, such as CD4 T-cells (Helper T-cells), CD8 T-cells (Cytotoxic T-cells, CTLs), regulatory T cells (T_{reg} cells) and memory T cells or memory stem cell T cells. In another embodiment, T-cells include Natural Killer T-cells (NK T-cells).

According to an embodiment of the present invention, T cells and NK cells useful in the present invention can be expanded and transfected with CAR polynucleotides in accordance to the present disclosure. T cells and NK cells can be derived from cord blood, peripheral blood, iPS cells and embryonic stem cells. According to one aspect of the present disclosure, T-cells cells may be expanded and transfected with CAR. CAR expressing T-cells can be expanded in serum free-medium with or without co-culturing with feeder cells. A pure population of T cells expressing the CAR of interest may be obtained by sorting, for example by utilizing a truncated epidermal growth factor receptor (EGFRt) which is linked to a cleavable peptide such as P2A and sorting the appropriate T cells using an anti-EGFRt antibody. Pursuant to this approach, P2A should get cleaved during/after protein translation, and EGFRt should be expressed in cis with the SMART-CAR on the cell surface (not attached as part of the same polypeptide chain). Being expressed in cis allows it to be used as an expression and selection marker without interfering with the SMART-CAR.

In some embodiments, the engineered cell may be modified to prevent expression of cell surface antigens.

In some embodiments, the engineered cell includes an inducible suicide gene (“safety switch”) or a combination of safety switches, which may be assembled on a vector, such as, without limiting, a retroviral vector, lentiviral vector, adenoviral vector or plasmid. It is noted that the CAR polypeptides which are conjugated to bi-functional molecules according to the present invention increase the safety profile of the therapy because of the specific targeting of cancer cells. Notwithstanding the clear advance of the present invention to use a small molecule cancer targeting moiety conjugated to the CAR T cell, introduction of a “safety switch” in the CAR polypeptide may further increase the safety profile and limit on-target or off-tumor toxicities of the compound CARs. The “safety switch” may be an inducible suicide gene, such as, without limiting, caspase 9 gene, thymidine kinase, cytosine deaminase (CD) or cytochrome P450. Other safety switches for elimination of unwanted modified T cells involve expression of CD20 or CD52 or CD19 or truncated epidermal growth factor receptor in T cells. All possible safety switches have been contemplated and are embodied in the present disclosure. In some embodiments, the suicide gene is integrated into the engineered cell genome.

In particular embodiments, the engineered cell includes a CAR linked to EGFRt via the P2 Å cleavage sequence as indicated in FIG. 5. A polypeptide providing this embodiment is included with the DNA constructs for CAR7 and CAR13 of FIG. 5 hereof.

In particular embodiments, the engineered cell includes CAR linked to 4-1BBL (CD137L) via a hinge sequence. A polypeptide providing a CAR with a C28 linked to 4-1BBL this embodiment includes CAR7 and CAR13 of FIG. 5 and FIG. 24.

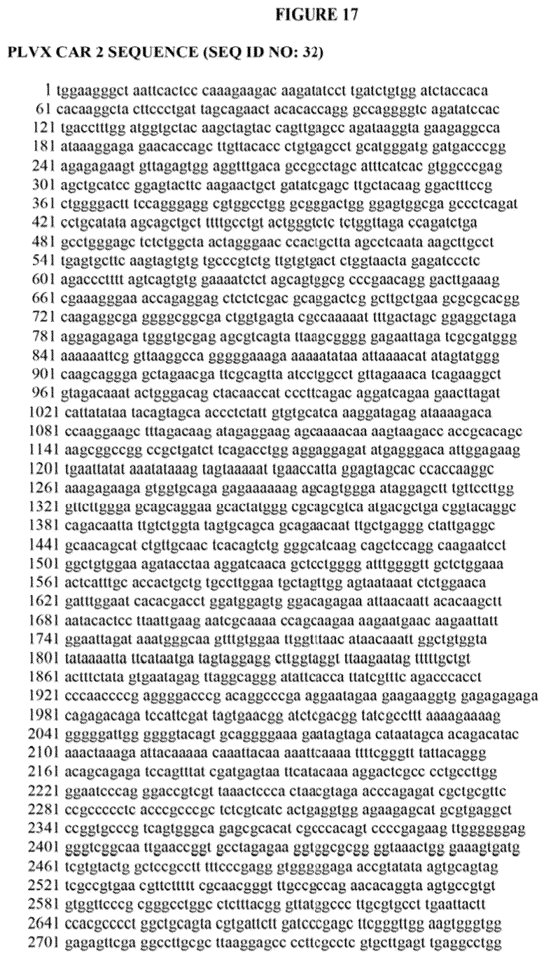

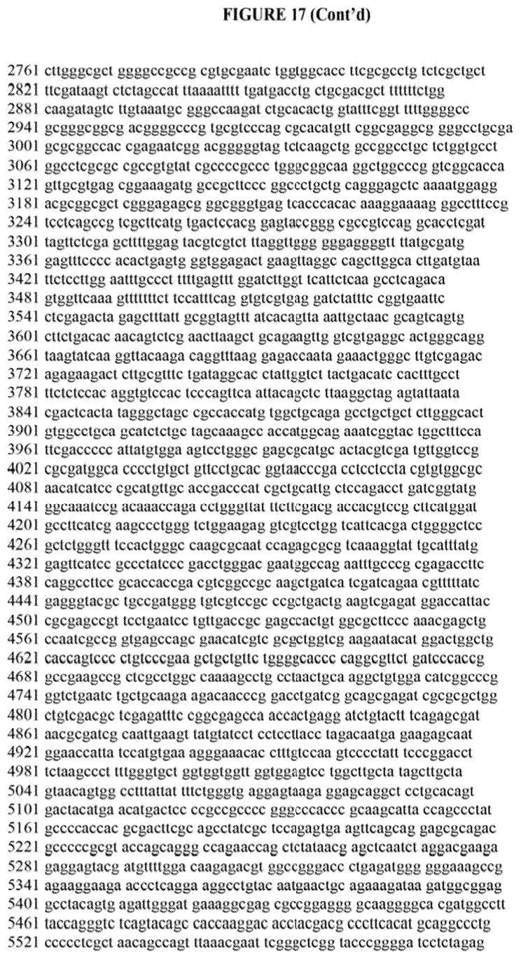

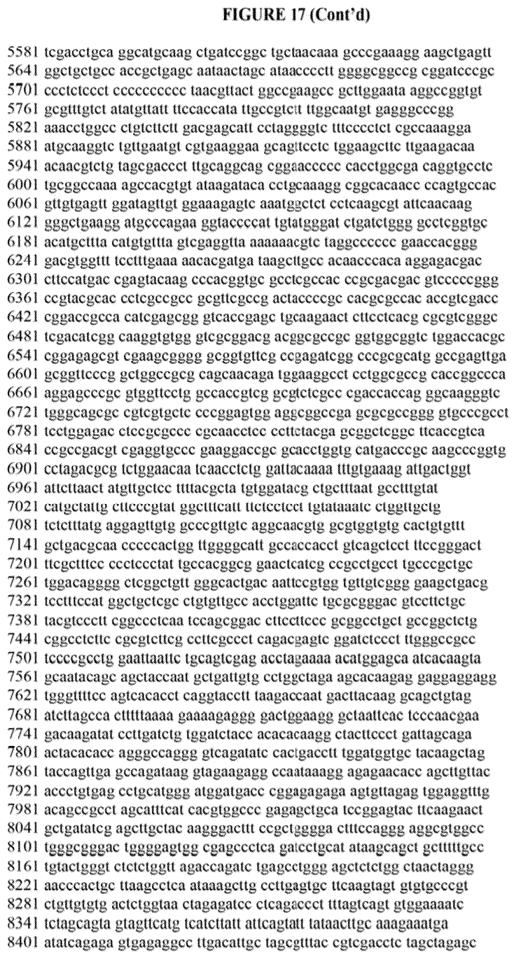

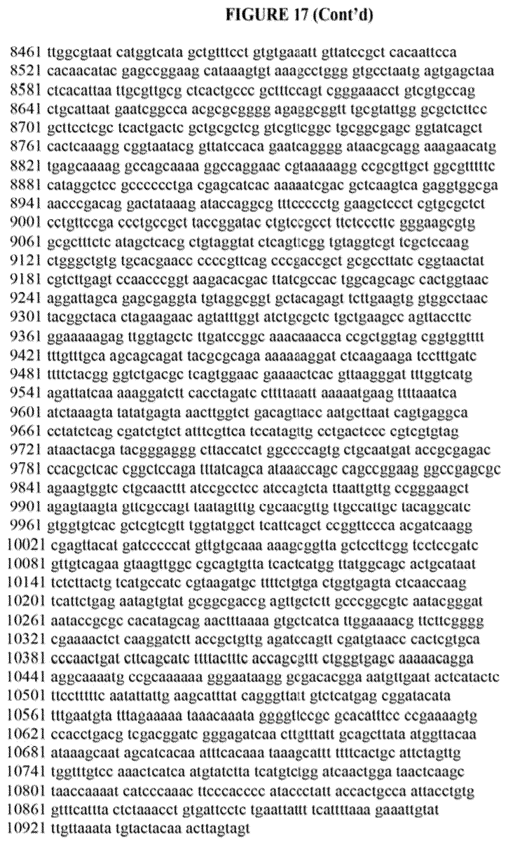

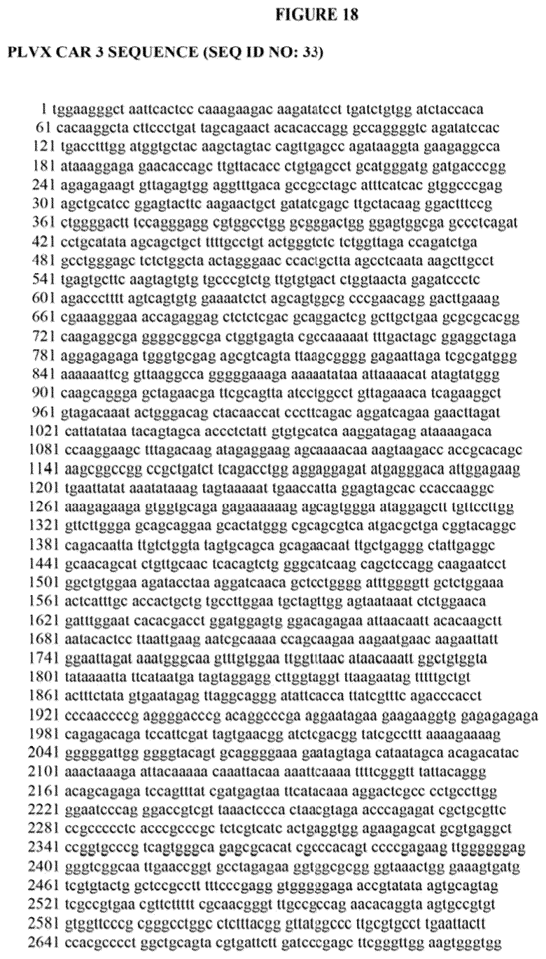

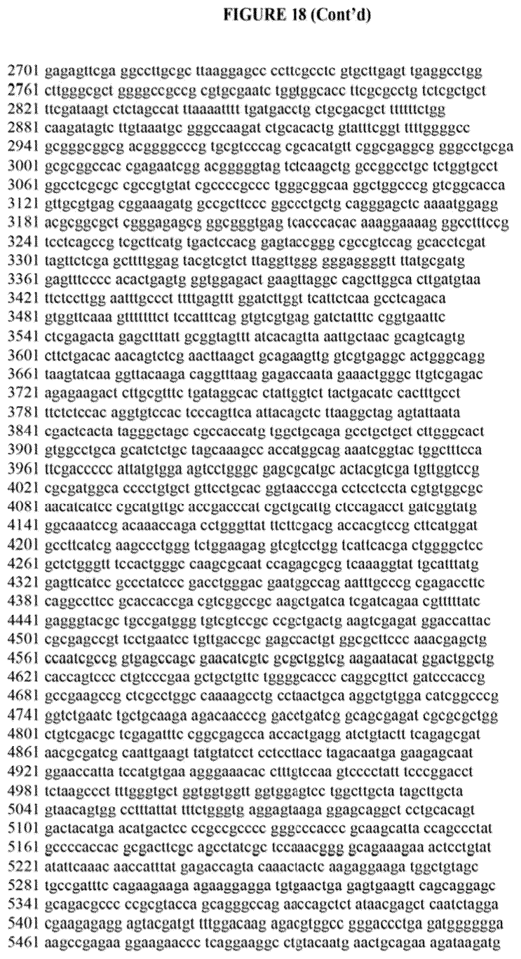

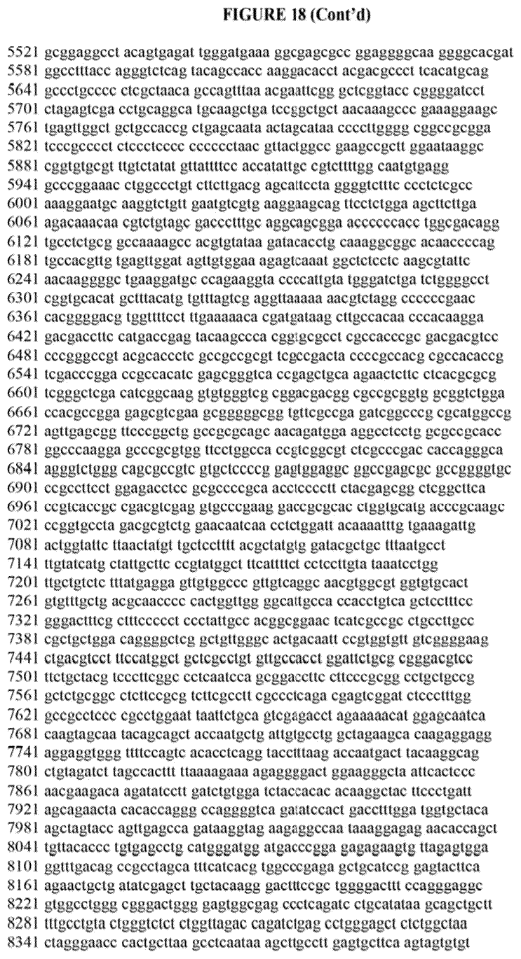

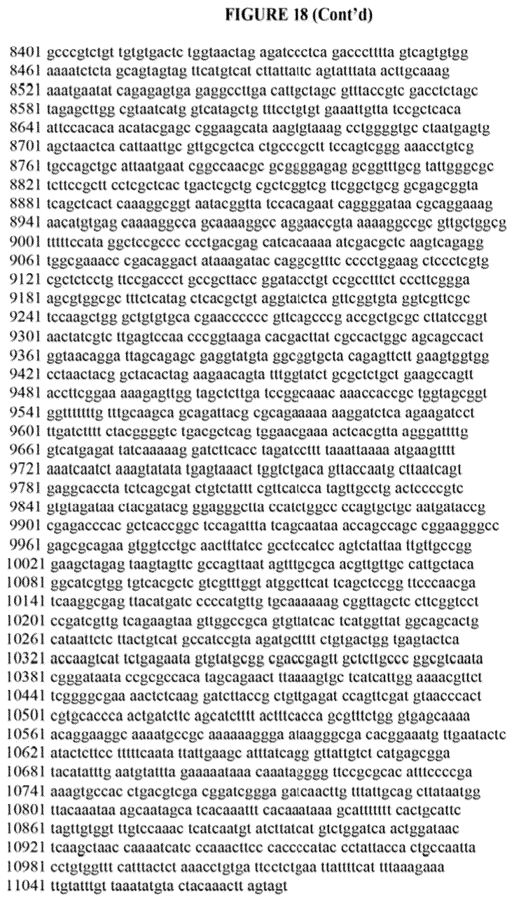

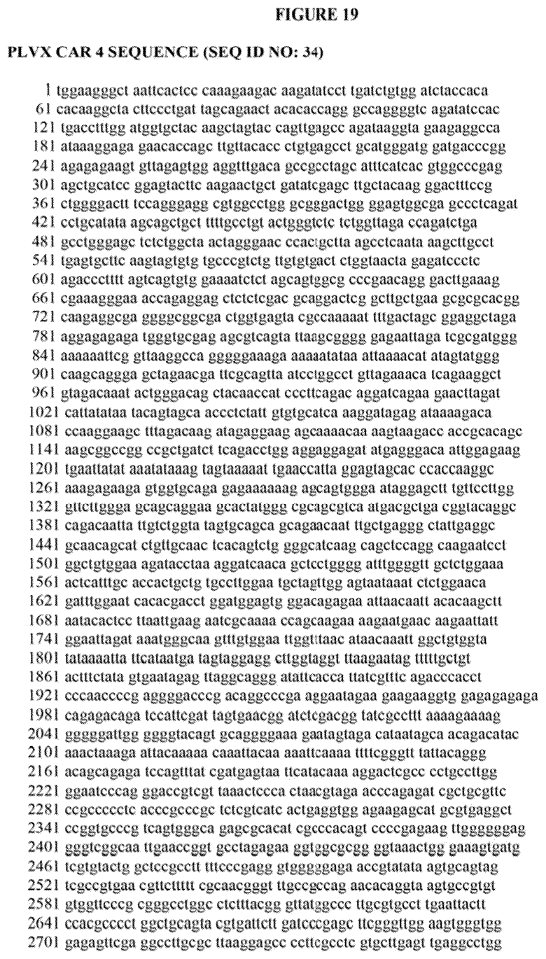

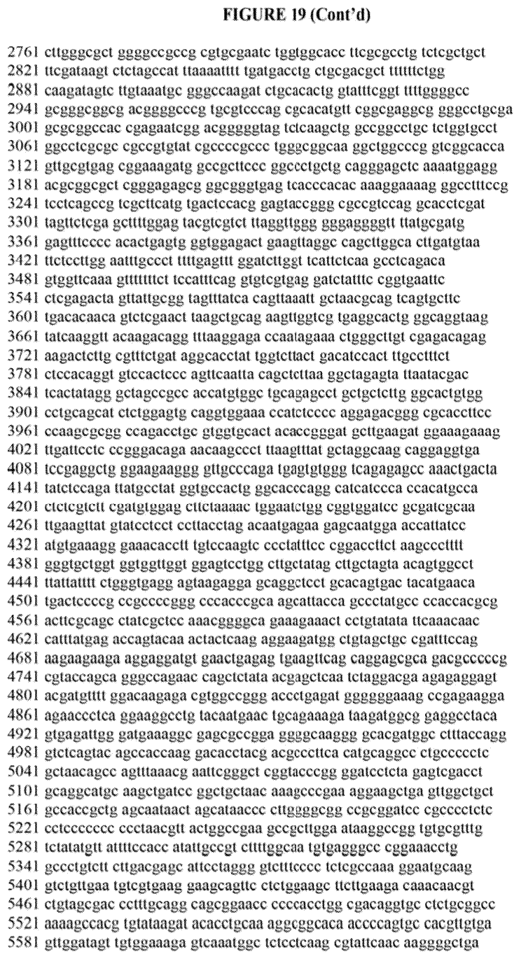

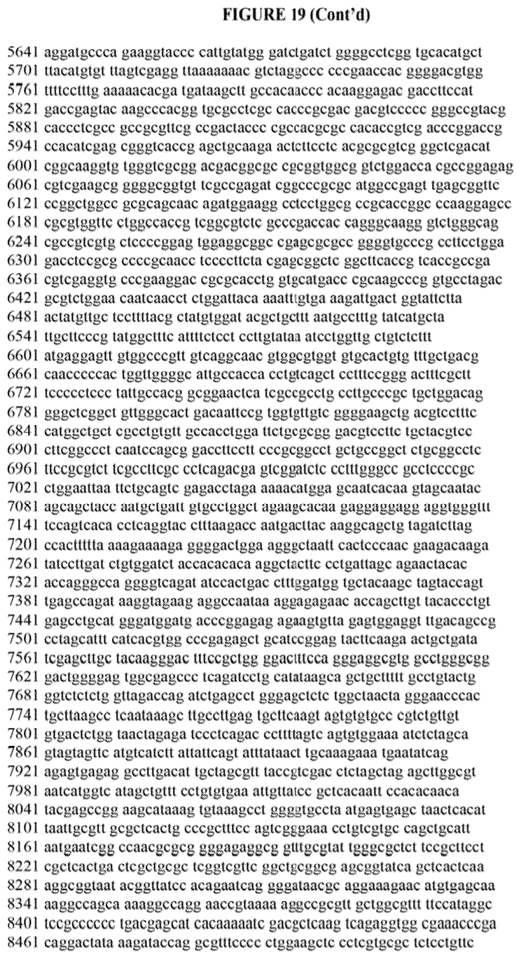

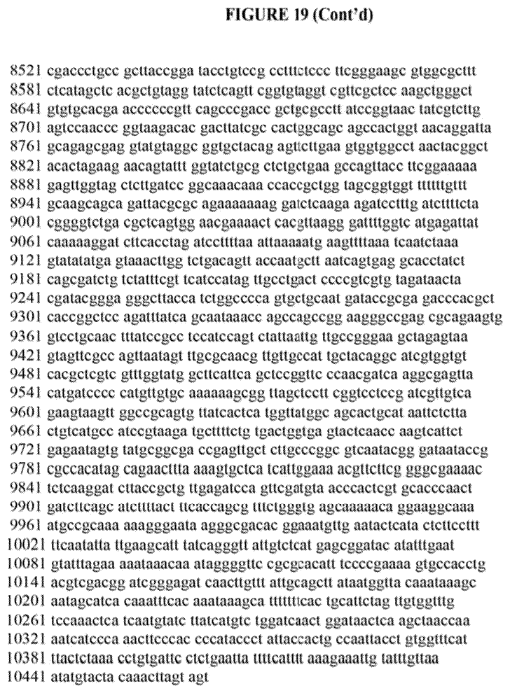

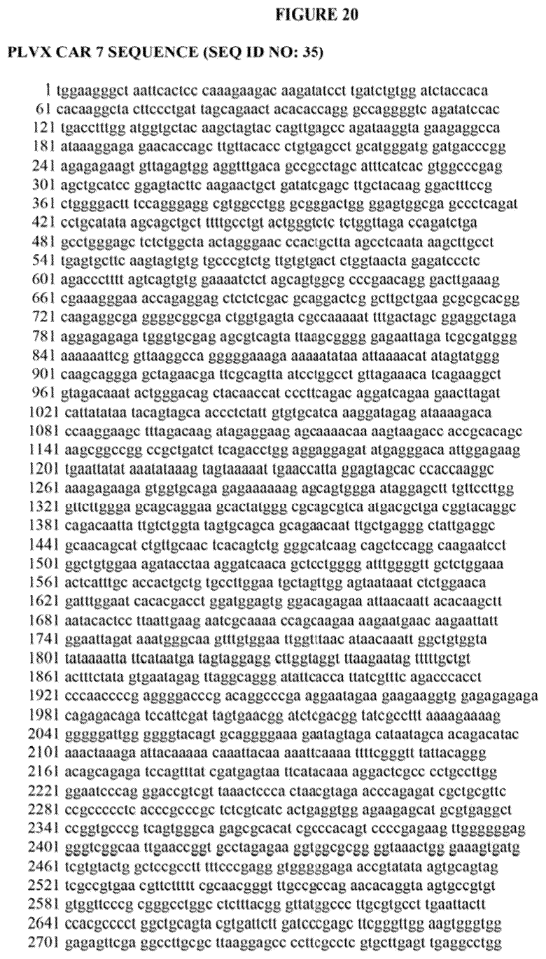

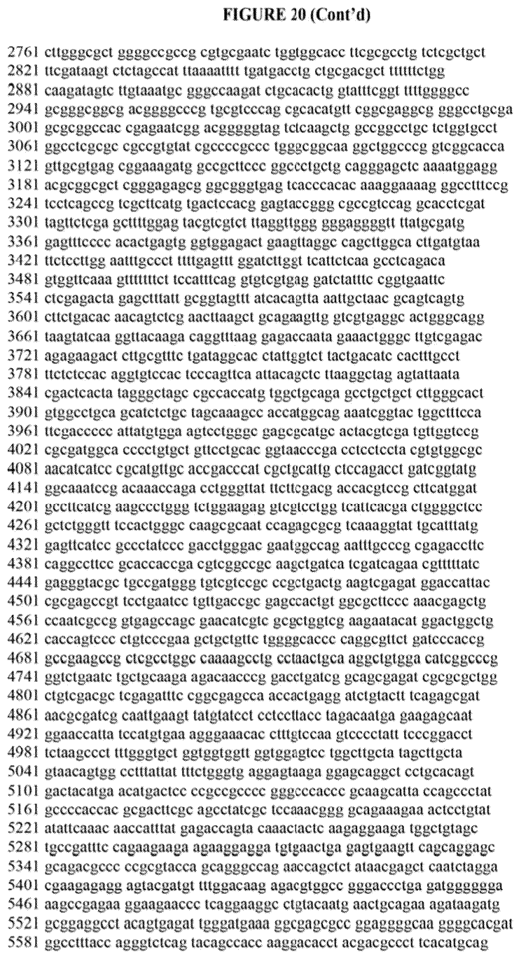

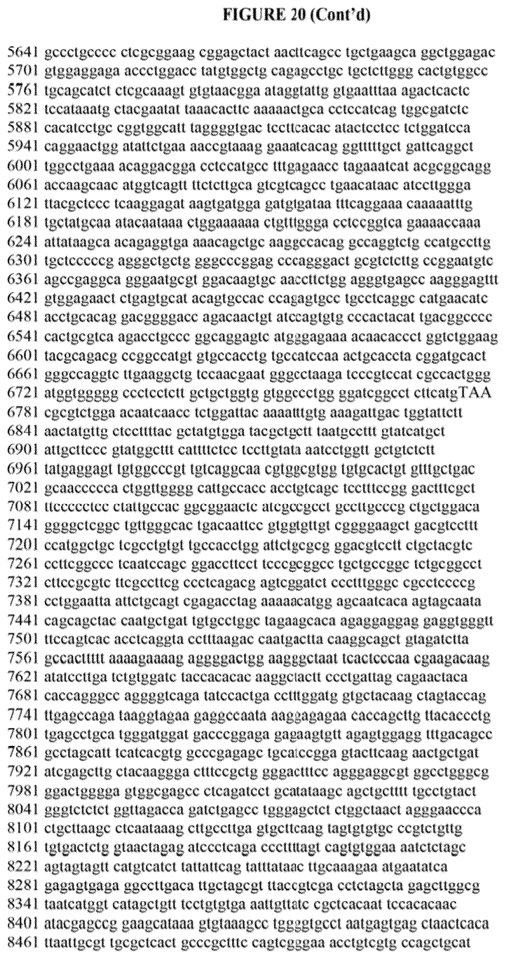

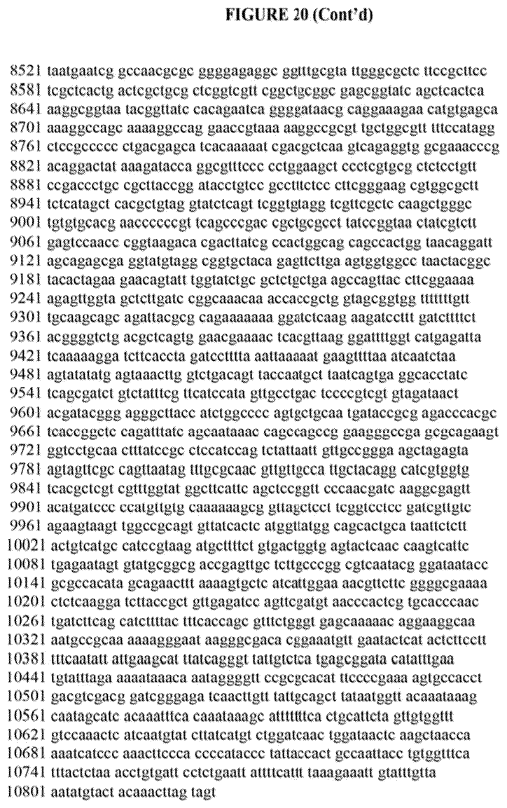

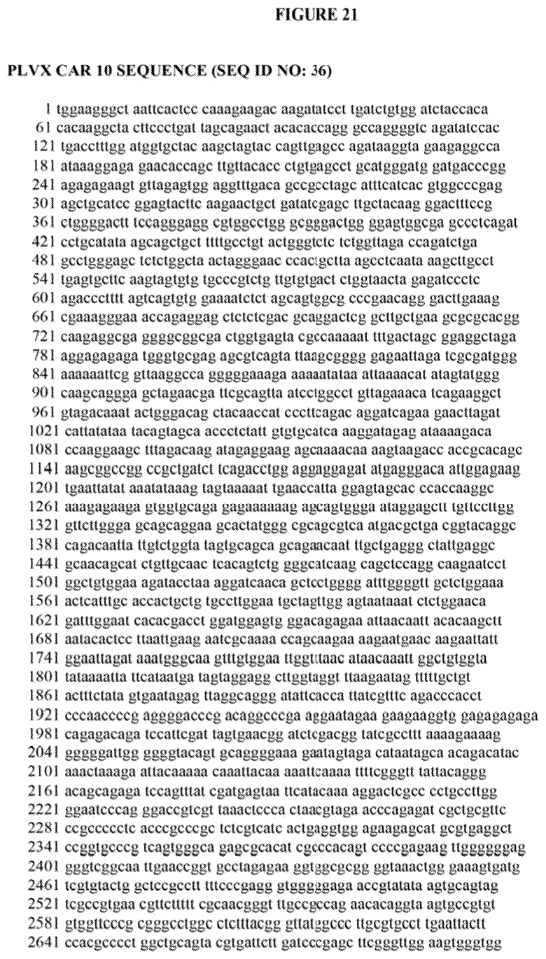

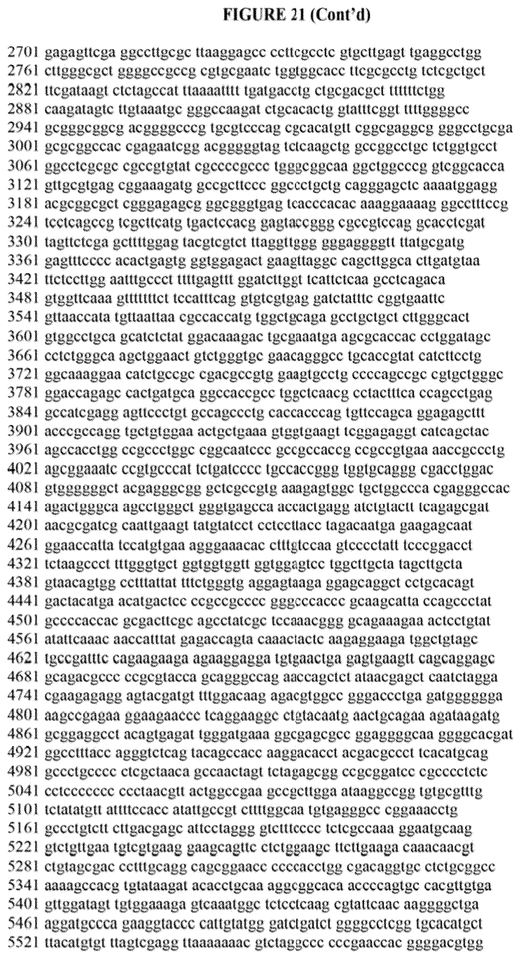

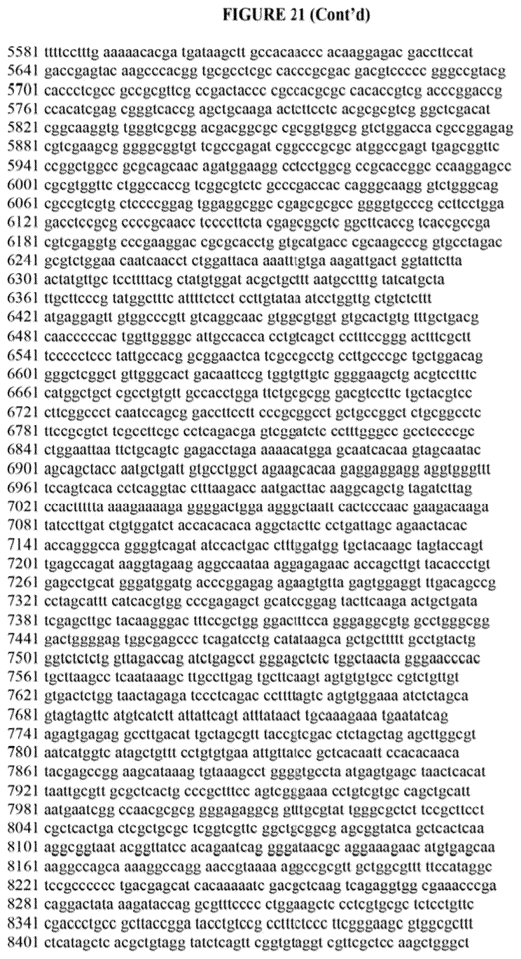

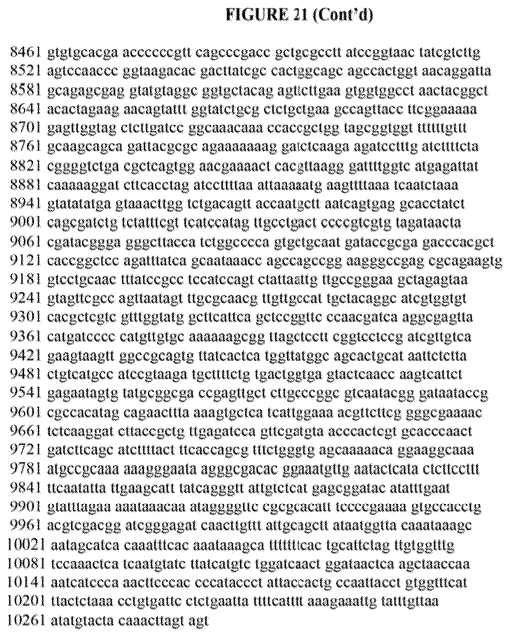

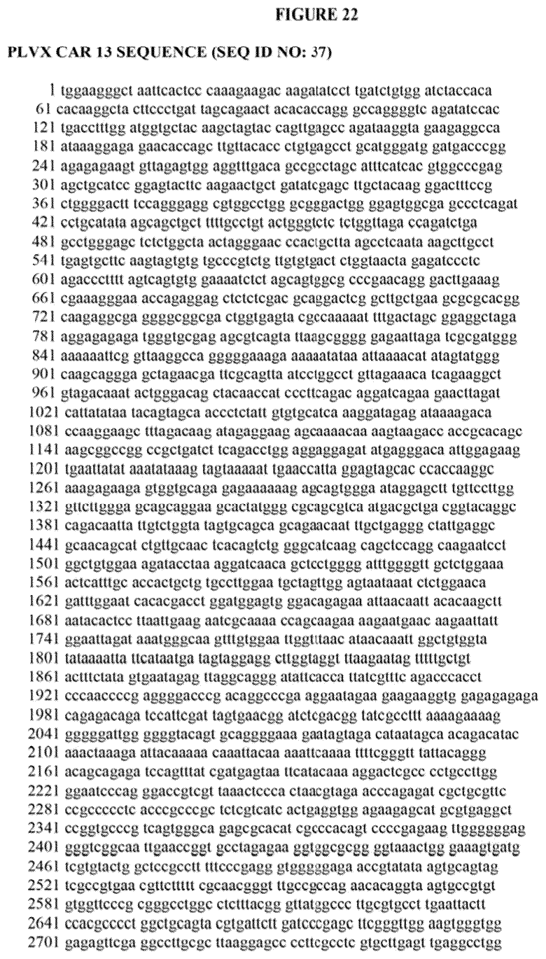

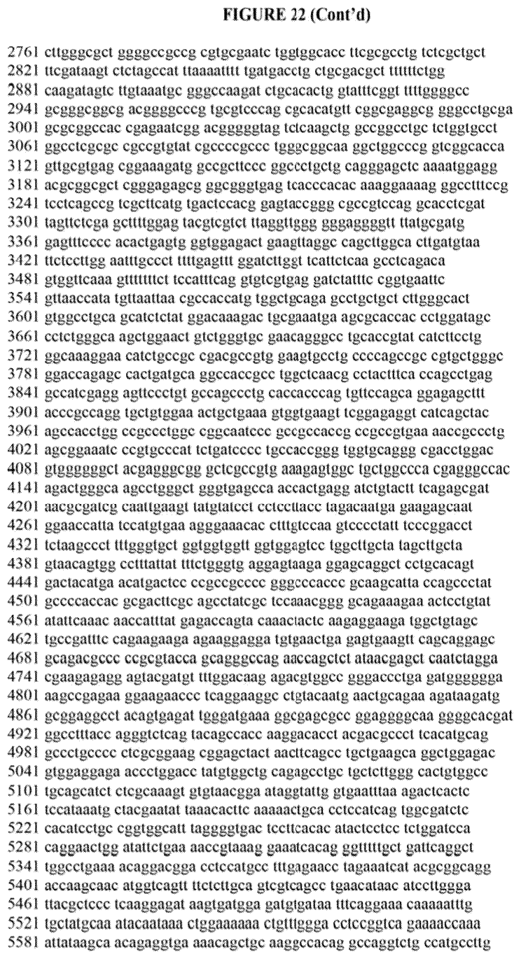

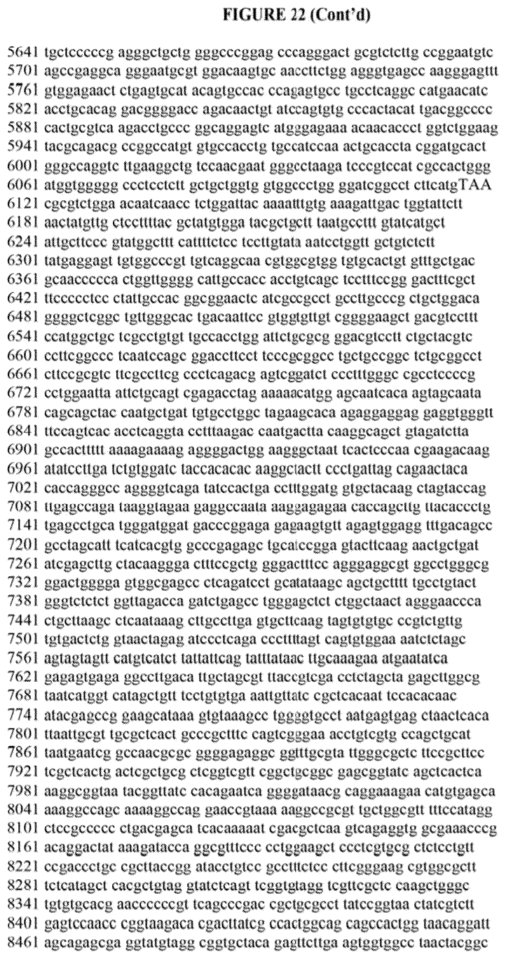

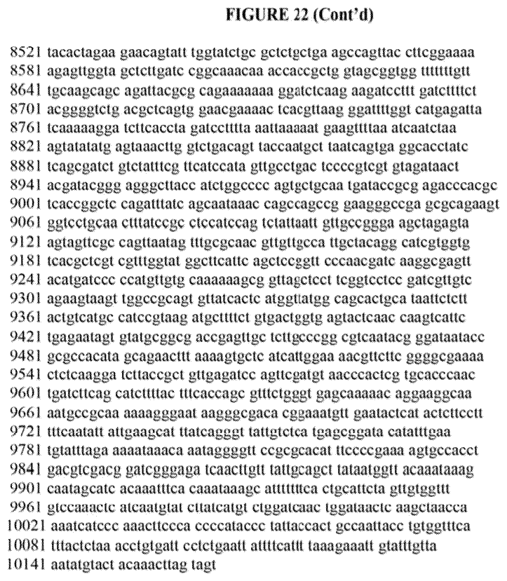

A number of DNA sequences for the PLVX vectors for CAR 1, CAR 2, CAR 3, CAR 4, CAR 7, CAR 10 and CAR 13 of FIG. 5 are presented respectively, in FIG. 16 (SEQ ID NO: 31), FIG. 17 (SEQ ID NO: 32), FIG. 18 (SEQ ID NO: 33), FIG. 19 (SEQ ID NO: 34), FIG. 20 (SEQ ID NO: 35), FIG. 21 (SEQ ID NO: 36) and FIG. 22 (SEQ ID NO: 37).

The term “co-administration” shall mean that at least two compounds or compositions are administered to the patient at the same time, such that effective amounts or concentrations of each of the two or more compounds may be found in the patient at a given point in time. Although CAR T cell-bifunctional molecule conjugates (SMART CARs) according to the present invention may be co-administered to a patient at the same time, the term embraces both administration of two or more agents at the same time or at different times, provided that effective concentrations of all co-administered compounds or compositions are found in the subject at a given time.

CAR T cell-bifunctional molecule conjugates according to the present invention may be administered with one or more additional anti-cancer agents or other agents which are used to treat or ameliorate the symptoms of cancer, especially prostate cancer, including metastatic prostate cancer. Exemplary anticancer agents which may be co-administered in combination with one or more CAR T cell-bifunctional molecule conjugates according to the present invention include, for example, antimetabolites, inhibitors of topoisomerase I and II, alkylating agents and microtubule inhibitors (e.g., taxol). Specific anticancer compounds for use in the present invention include, for example, Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others.

In addition to anticancer agents, a number of other agents may be co-administered with chimeric antigen receptor (CAR) T cell-bi-functional molecule conjugates according to the present invention in the treatment of cancer, especially prostate cancer, including metastatic prostate cancer. These include active agents, minerals, vitamins and nutritional supplements which have shown some efficacy in inhibiting prostate cancer tissue or its growth or are otherwise useful in the treatment of prostate cancer. For example, one or more of dietary selenium, vitamin E, lycopene, soy foods, vitamin D, green tea, lycopene, omega-3 fatty acids and phytoestrogens, including beta-sitosterol, may be utilized in combination with the present compounds to treat prostate cancer.

In addition, active agents, other than traditional anticancer agents have shown some utility in treating prostate cancer. The selective estrogen receptor modulator drug toremifene may be used in combination with the present compounds to treat cancer, especially prostate cancer, including metastatic prostate cancer. In addition, two medications which block the conversion of testosterone to dihydrotestosterone, finasteride and dutasteride, are also useful in the treatment of prostate cancer when coadministered with compounds according to the present invention. The phytochemicals indole-3-carbinol and diindolylmethane, may also be coadministered with the present compounds for their effects in treating prostate cancer. Additional agents which may be combined with compounds according to the present invention include antiandrogens, for example, flutamide, bicalutamide, nilutamide, and cyproterone acetate as well as agents which reduce the production of adrenal androgens (e.g. DHEA), such as ketoconazole and aminoglutethimide. Other active agents which may be combined with compounds according to the present invention include, for example, GnRH modulators, including agonists and antagonists. GnRH antagonists suppress the production of LH directly, while GnRH agonists suppress LH through the process of downregulation after an initial stimulation effect. Abarelix is an example of a GnRH antagonist, while the GnRH agonists include leuprolide, goserelin, triptorelin, and buserelin, among others. These agents may be combined with compounds according to the present invention in effective amounts. In addition, abiraterone acetate may also be combined with one or more compounds according to the present invention in the treatment of prostate cancer, especially including metastatic prostate cancer.

Other agents which may be combined with one or more chimeric antigen receptor (CAR) T cell-bi-functional molecule conjugates according to the present invention, include the bisphosphonates such as zoledronic acid, which have been shown to delay skeletal complications such as fractures which occur with patients having metastatic prostate cancer. Alpharadin, another agent, may be combined with compounds according to the present invention to target bone metastasis. In addition, bone pain due to metastatic prostate cancer may be treated with opioid pain relievers such as morphine and oxycodone, among others, which may be combined with compounds according to the present invention.

Pharmaceutical compositions comprising combinations of an effective amount of at least one chimeric antigen receptor (CAR) T cell-bi-functional molecule conjugates, all in effective amounts, in combination with a pharmaceutically effective amount of a carrier, additive or excipient, represents a further aspect of the present invention.

The chimeric antigen receptor (CAR) T cell-bi-functional molecule conjugates of the present invention may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The CAR T cell-bifunctional molecule conjugates of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered parenterally, including intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially to treat skin cancers, psoriasis or other diseases which occur in or on the skin. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and disease treated, the particular mode of administration. Preferably, the compositions (generally, bi-functional compounds and/or additional anticancer agents as described herein) should be formulated to contain between about 0.05 milligram to about several grams (e.g. 2-3 grams up to 5 grams or more), about 0.1 milligram to about 750 milligrams or more (2-3 grams), more preferably about 1 milligram to about 600 milligrams, and even more preferably about 10 milligrams to about 500 milligrams of active ingredient as small molecules. SMART CAR T cells, alone or in combination with at least one additional compound may be used to treat cancer, prostate cancer or metastatic prostate cancer or a secondary effect or condition thereof. The bifunctional molecules and the CAR T cells may be delivered together. Preferably, the bi-functional molecules and the additional anticancer compounds delivered separately from the CAR T cells and by separate mechanisms. The cell component of CAR-T cells is generally measured in cell number and administered as such, such as from 1E5-1E8 cells/kg, 1E5-1E7 cells/kg., more often 1E6 cells/kg etc. The cells are often delivered parenterally, especially including intravenously.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject (e.g. a male human) suffering from cancer can be treated by administering to the patient (subject) an effective amount of chimeric antigen receptor (CAR) T cell-bi-functional molecule conjugates according to the present invention including pharmaceutically acceptable salts, solvates or polymorphs, thereof optionally in a pharmaceutically acceptable carrier or diluent, either alone, or in combination with other known anticancer or pharmaceutical agents, preferably agents which can assist in treating prostate cancer, including metastatic prostate cancer or ameliorate the secondary effects and conditions associated with prostate cancer. This treatment can also be administered in conjunction with other conventional cancer therapies, such as radiation treatment or surgery.

The method of treatment may further comprise such steps as T cell apheresis, retroviral or lentiviral CAR transduction, T cell expansion, and host conditioning which are performed before administration of the chimeric antigen receptor (CAR) T cell-bi-functional molecule conjugates to the subject.

The chimeric antigen receptor (CAR) T cell-bi-functional molecule conjugates can be administered by any appropriate route, for example, orally, parenterally, intravenously, intradermally, subcutaneously, or topically, in liquid, cream, gel, or solid form, or by aerosol form. Preferably parenteral administration is used, especially intravenous administration.

The active composition is included in the pharmaceutically acceptable carrier or diluent in an amount sufficient to deliver to a patient a therapeutically effective amount for the desired indication, without causing serious toxic effects in the patient treated. A preferred dose of the active composition for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from 0.01-3% wt/wt in a suitable carrier.

The composition is conveniently administered in any suitable unit dosage form, including but not limited to one containing less than 1 mg, 1 mg to 3000 mg or more, preferably 5 to 500 mg of active ingredient per unit dosage form.

The active ingredient is preferably administered to achieve peak plasma concentrations of the active composition of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient.

The concentration of active composition in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions, when used, will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active composition or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active composition or pharmaceutically acceptable salts thereof can also be mixed with other active materials that do not impair the desired action, or with materials that supplement the desired action, such as other anticancer agents, antibiotics, antifungals, antiinflammatories, or antiviral compounds. In certain preferred aspects of the invention, one or more chimeric antibody-recruiting compound according to the present invention is coadministered with another anticancer agent and/or another bioactive agent, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compositions are then introduced into the container. The container is then swirled by hand to free lipid material from the sides of the container and to disperse lipid aggregates, thereby forming the liposomal suspension.

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; TRL.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95, 96, 97, 98, 98.5, 99, or 99.5% of the aligned sequences. Preferably, the homology is over a full-length sequence, or a protein thereof, or a fragment thereof which is at least 8 amino acids, or more desirably, at least 15 amino acids in length. Examples of suitable fragments are described herein.

The term “percent sequence identity” or “identical” in the context of nucleic acid sequences refers to the residues in the two sequences that are the same when aligned for maximum correspondence. The length of sequence identity comparison may be over the full-length of the genome (e.g., about 36 kbp), the full-length of an open reading frame of a gene, protein, subunit, or enzyme [see, e.g., the sequences provided in FIGS. 16-22 providing the lentiviral coding sequences], or a fragment of at least about 500 to 5,000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length, and may be up to about 700 amino acids. Examples of suitable fragments are described herein. In one embodiment, there is amino acid identity in at least about 95, 96, 97, 98, 98.5, 99 or 99.5% of the aligned sequences.

A “promoter sequence” is a DNA regulatory region capable of binding RNA polymerase in a cell and initiating transcription of a downstream (3 direction) coding sequence. For purposes of defining the present invention, the promoter sequence is bounded at its 3 terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

A “signal sequence” or “signal peptide” can be included before the coding sequence. This sequence encodes a signal peptide, often inserted N-terminal to the (CAR) polypeptide, or N-terminal to a particular component of a CAR that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes. In preferred embodiments according to the present invention, the signal sequence used in CAR expression vectors is the first 17AA of and is generally inserted nearer the amino terminus than the CAR polypeptide and in particular, the antigen binding region. In a preferred embodiment, the signal peptide is the first 17 AA of GMCSF, MWLQSLLLLGTVACSIS, SEQ ID No: 8, which is encoded by the polynucleotide ATGTGGCTGCAGAGCCTGCTGCTCTTGGGCACTGTGGCCTGCAGCATCTCT,

SEQ ID. NO: 55. Numerous additional signal sequences can be used in the present invention, including, for example, a human signal peptide of human protein of type I, II, Ill, or IV, including an immunoglobulin heavy chain signal peptide, the signal peptide from human CD45 (UniProtKB/Swiss-Prot Accession Number P08575), which is 23 amino acids in length (MYLWLKLLAFGFAFLDTEVFVTG, SEQ ID. NO: 9) or a functional fragment thereof, which includes a fragment of at least 10 amino acids of the CD45 signal peptide that directs the appended polypeptide to the cell membrane and cell surface. Examples of fragments of the human CD45 signal peptide which may be used in the present invention include: MYLWLKLLAFG, SEQ ID. NO: 10, FAFLDTEVFVTG, SEQ ID. NO: 11 and LKLLAFGFAFLDTE, SEQ ID. NO: 12.

Functional equivalents of the human CD45 signal peptide have also been contemplated. As used herein, “functional equivalents” are to be understood as mutants that exhibit, in at least one of the abovementioned sequence positions, an amino acid substitution other than the one mentioned specifically, but still lead to a mutant which show the same or similar properties with respect to the wild-type CD45 signal peptide. Functional equivalents of these signal peptides include polypeptides having at least 80%, at least 85%, at least 90%, or at least 95% identity to the human CD45 signal peptide, functional fragments thereof, or functional equivalents thereof. Functional equivalents also include CD45 signal peptides from homologous proteins from other species. Examples of these signal peptides include signal peptide from mouse CD45 (MGLWLKLLAFGFALLDTEVFVTG, SEQ ID. No: 13); signal peptide from rat CD45 (MYLWLKLLAFSLALLGPEVFVTG, SEQ ID. No: 14); signal peptide from sheep CD45 (MTMYLWLKLLAFGFAFLDTAVSVAG, SEQ ID NO: 15); signal peptide from chimpanzee CD45 (MYLWLKLLAFGFAFLDTEVFVTG, SEQ ID NO:16); and signal peptide from monkey CD45 (MTMYLWLKLLAFGFAFLDTEVFVAG, SEQ ID NO:17).

The signal peptide may also include the signal peptide from human CD8a (MALPVTALLLPLALLLHAARP, SEQ ID NO: 18). In some embodiments, the signal peptide may be a functional fragment of the CD8a signal peptide. A functional fragment includes a fragment of at least 10 amino acids of the CD8a signal peptide that directs the appended polypeptide to the cell membrane and cell surface. Examples of fragments of the human CD8a signal peptide include: MALPVTALLLPLALLLHAA SEQ ID NO:19, MALPVTALLLP SEQ ID NO:20, PVTALLLPLALL SEQ ID NO:21, and LLLPLALLLHAARP, SEQ ID NO:22. In another embodiment, the signal peptide includes the signal peptide from human CD8b (MRPRLWLLLAAQLTVLHGNSV, SEQ ID NO:23). In some embodiments, the signal peptide may be a functional fragment of the CD8b signal peptide. A functional fragment includes a fragment of at least 10 amino acids of the CD8b signal peptide that directs the appended polypeptide to the cell membrane and cell surface. Examples of fragments of the human CD8b signal peptide include: MRPRLWLLLAAQ, SEQ ID NO: 24, RLWLLLAAQLTVLHG, SEQ ID NO: 25, and LWLLLAAQLTVLHGNSV, SEQ ID NO: 26.

Functional equivalents of the human CD8a or CD8b signal peptide may also be used and these are to be understood as mutants which exhibit, in at least one of the abovementioned sequence positions, an amino acid substitution other than the one mentioned specifically, but still lead to a mutant which show the same or similar properties with respect to the wild-type CD8a or CD8b signal peptide. Functional equivalents include polypeptides having at least 80%, at least 85%, at least 90%, or at least 95% identity to the human CD8 signal peptide, functional fragments thereof, or functional equivalents thereof. Functional equivalents also include CD8a and CD8b signal peptides from homologous proteins from other species.

Additional signal peptides for use in the present invention include the signal peptide from human IL-2. The IL-2 signal peptide is 23 amino acids in length (MYRMQLLSCIALSLALVTNS, SEQ ID NO: 27). In some embodiments, the signal peptide may be a functional fragment of the IL-2 signal peptide. A functional fragment includes a fragment of at least 10 amino acids of the IL-2 signal peptide that directs the appended polypeptide to the cell membrane and cell surface. Examples of fragments of the human IL-2 signal peptide include: MYRMQLLSCIAL SEQ ID NO: 28, QLLSCIALSLAL SEQ ID NO: 29, and SCIALSLALVTNS SEQ ID NO: 30. Functional equivalents of the human IL-2 signal peptide have also been contemplated. As used herein, “functional equivalents” are to be understood as mutants which exhibit, in at least one of the abovementioned sequence positions, an amino acid substitution other than the one mentioned specifically, but still lead to a mutant which show the same or similar properties with respect to the wild-type IL-2 signal peptide. Functional equivalents include polypeptides having at least 80%, at least 85%, at least 90%, or at least 95% identity to the human IL-2 signal peptide, functional fragments thereof, or functional equivalents thereof.

When a transmembrane protein is being translated, the signal peptide is the first thing that emerges from the ribosome. The signal peptide (also referred to as the signal sequence) gets recognized by the signal recognition partical (SRP), which recruits it to the endoplasmic reticulum (ER) membrane for translocation into the ER. For most transmembrane proteins, the signal sequence will get cleaved off upon completion of translocation into the ER. From there, the protein will traffic through the golgi apparatus to the cell membrane.

The signal sequence often gets cleaved and is generally not relevant to the expressed CAR polypeptide function. In embodiments where the signal sequence does not get cleaved, it generally does not interfere with the CAR polypeptide expression, or the functioning of the polypeptide, including its binding dynamics. The principal purpose of the signal sequence for use in the present invention to cause the CAR receptor to traffic to the cell membrane, and is should be largely interchangeable with any other characterized transmembrane proteins.

A nucleic acid molecule is “operatively linked” to, or “operably associated with”, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

Nucleic acid sequences of the invention may include nucleic acid sequences that encode a reporter polypeptide, e.g. a MRI reporter, a PET reporter; a SPECT reporter, a photoacoustic reporter, a bioluminescent reporter, or any combination thereof.

A level and/or an activity and/or expression of a translation product of a gene and/or of a fragment, or derivative, or variant of said translation product, and/or the level or activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

Certain diagnostic and screening methods of the present invention utilize an antibody, preferably, a monocolonal antibody, capable of specifically binding to a protein as described herein or active fragments thereof. The method of utilizing an antibody to measure the levels of protein allows for non-invasive diagnosis of the pathological states of kidney diseases. In a preferred embodiment of the present invention, the antibody is human or is humanized. The preferred antibodies may be used, for example, in standard radioimmunoassays or enzyme-linked immunosorbent assays or other assays which utilize antibodies for measurement of levels of protein in sample. In a particular embodiment, the antibodies of the present invention are used to detect and to measure the levels of protein present in a sample.

Humanized antibodies are antibodies, or antibody fragments, that have the same binding specificity as a parent antibody, (i.e., typically of mouse origin) and increased human characteristics. Humanized antibodies may be obtained, for example, by chain shuffling or by using phage display technology. For example, a polypeptide comprising a heavy or light chain variable domain of a non-human antibody specific for a disease related protein is combined with a repertoire of human complementary (light or heavy) chain variable domains. Hybrid pairings specific for the antigen of interest are selected. Human chains from the selected pairings may then be combined with a repertoire of human complementary variable domains (heavy or light) and humanized antibody polypeptide dimers can be selected for binding specificity for an antigen. Techniques described for generation of humanized antibodies that can be used in the method of the present invention are disclosed in, for example, U.S. Pat. Nos. 5,565,332; 5,585,089; 5,694,761; and 5,693,762. Furthermore, techniques described for the production of human antibodies in transgenic mice are described in, for example, U.S. Pat. Nos. 5,545,806 and 5,569,825.

CAR T cells and conjugate bi-functional molecules can also be labeled with fluorophores including small molecule fluors and proteinaceous fluors (e.g. green fluorescent proteins and derivatives thereof). Useful fluorophores include, but are not limited to, 1,1′-diethyl-2,2′-cyanine iodide, 1,2-diphenylacetylene, 1,4-diphenylbutadiene, 1,6-Diphenylhexatriene, 2-Methylbenzoxazole, 2,5-Diphenyloxazole (PPO), 4-(dicyanomethylene)-2-methyl-6-(p-dimethylaminostyryl)-4H-pyran (DCM), 4-Dimethylamino-4′-nitrostilbene, 4′,6-Diamidino-2-phenylindole (DAPI), 5-ROX, 7-AAD, 7-Benzylamino-4-nitrobenz-2-oxa-1,3-diazole, 7-Methoxycoumarin-4-acetic acid, 9,10-Bis(phenylethynyl)anthracene, 9,10-Diphenylanthracene, Acridine Orange, Acridine yellow, Adenine, Allophycocyanin (APC), AMCA, AmCyan, Anthracene, Anthraquinone, APC, Auramine O, Azobenzene, Benzene, Benzoquinone, Beta-carotene, Bilirubin, Biphenyl, BO—PRO-1, BOBO-1, BODIPY FL, Calcium Green-1, Cascade Blue™, Cascade Yellow™, Chlorophyll a, Chlorophyll b, Chromomycin, Coumarin, Coumarin 1, Coumarin 30, Coumarin 314, Coumarin 343, Coumarin 6, Cresyl violet perchlorate, Cryptocyanine, Crystal violet, Cy2, Cy3, Cy3.5, Cy5, Cy5.5, Cy7, Cytosine, DA, Dansyl glycine, DAPI, Dil, DiO, DiOCn, Diprotonated-tetraphenylporphyrin, DsRed, EDANS, Eosin, Erythrosin, Ethidium Monoazide, Ethyl p-dimethylaminobenzoate, FAM, Ferrocene, Fl, Fluo-3, Fluo-4, Fluorescein, Fluorescein isothiocyanate (FITC), Fura-2, Guanine, HcRed, Hematin, Histidine, Hoechst, Hoechst 33258, Hoechst 33342, IAEDANS, Indo-1, Indocarbocyanine (C3) dye, Indodicarbocyanine (C5) dye, Indotricarbocyanine (C7) dye, LC Red 640, LC Red 705, Lucifer yellow, LysoSensor Yellow/Blue, Magnesium octaethylporphyrin, Magnesium octaethylporphyrin (MgOEP), Magnesium phthalocyanine (MgPc), Magnesium tetramesitylporphyrin (MgTMP), Magnesium tetraphenylporphyrin (MgTPP), Malachite green, Marina Blue®, Merocyanine 540, Methyl-coumarin, MitoTracker Red, N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, Naphthalene, Nile Blue, Nile Red, Octaethylporphyrin, Oregon green, Oxacarbocyanine (C3) dye, Oxadicarbocyanine (C5) dye, Oxatricarbocyanine (C7) dye, Oxazine 1, Oxazine 170, p-Quaterphenyl, p-Terphenyl, Pacific Blue®, Peridinin chlorophyll protein complex (PerCP), Perylene, Phenol, Phenylalanine, Phthalocyanine (Pc), Pinacyanol iodide, Piroxicam, POPOP, Porphin, Proflavin, Propidium iodide, Pyrene, Pyronin Y, Pyrrole, Quinine sulfate, R-Phycoerythrin (PE), Rhodamine, Rhodamine 123, Rhodamine 6G, Riboflavin, Rose bengal, SNARF®, Squarylium dye Ill, Stains-all, Stilbene, Sulforhodamine 101, SYTOX Blue, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), tetramethylrhodamine, Tetraphenylporphyrin (TPP), Texas Red®(TR), Thiacarbocyanine (C3) dye, Thiadicarbocyanine (C5) dye, Thiatricarbocyanine (C7) dye, Thiazole Orange, Thymine, TO-PRO®-3, Toluene, TOTO-3, TR, Tris(2,2′-bipyridyl)ruthenium(II), TRITC, TRP, Tryptophan, Tyrosine, Uracil, Vitamin B12, YO—PRO-1, YOYO-1, Zinc octaethylporphyrin (ZnOEP), Zinc phthalocyanine (ZnPc), Zinc tetramesitylporphyrin (ZnTMP), Zinc tetramesitylporphyrin radical cation, and Zinc tetraphenylporphyrin (ZnTPP). Suitable optical dyes are described in the 1996 Molecular Probes Handbook by Richard P. Haugland, hereby expressly incorporated by reference.

Prostate specific membrane antigen (PSMA) is a unique membrane bound glycoprotein, which is overexpressed manifold on prostate cancer as well as the neovasculature of most of the solid tumors, but not in the vasculature of the normal tissues. This unique expression of PSMA makes it an important marker as well as a large extracellular target of imaging agents. PSMA can serve as target for delivery of therapeutic agents such as cytotoxins or radionuclides. PSMA has two unique enzymatic functions, folate hydrolase and NAALADase and found to be recycled like other membrane bound receptors through clathrin coated pits. The internalization property of PSMA leads one to consider the potential existence of a natural ligand for PSMA.

### Representative Syntheses of the Bi-Functional Molecules

Preferred bi-functional molecules belong to a class of glutamate urea compounds capable of inhibiting PSMA with high potency. PSMA binding increases have been correlated to the length of the linker regions connecting the two poles of the molecule. Click chemistry can be used to synthesize and assemble various component moieties of the bi-functional molecules, alternatively the free amine can be coupled with a carboxylic acid moiety or other electrophile to provide according to the present invention. See Sharpless and Manetsch, Expert Opinion on Drug Discovery 2006, 1, 525-538. Non-limiting representative syntheses of PSMA and linker portions of the bi-functional molecules are shown below. Those of ordinary skill in the art are able to vary these syntheses to make other bi-functional molecules as defined in the instant invention.

A cancer binding moiety (PBM) may be readily constructed as indicated in the scheme below. Compound 12 is readily synthesized from compound 11 using the steps described in the scheme below. Compound 12, 14 or the free carboxylic acid of compound 14 can be condensed onto a proparyl group of an intermediate to form a triazole intermediate or final bi-functional molecule. The following are representative syntheses of components which may be used to provide bi-functional compounds according to the present invention.

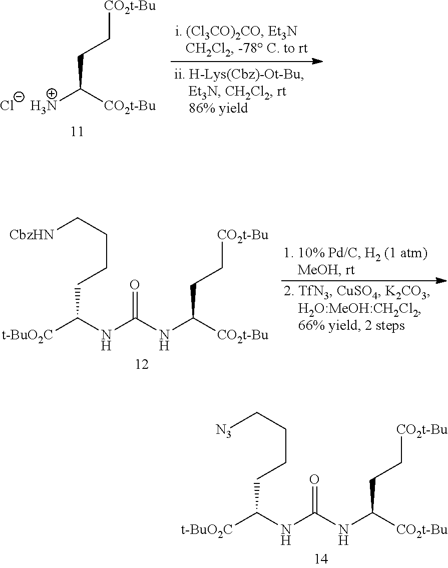

### (9S,13S)-tri-tert-butyl 3,11-dioxo-1-phenyl-2-oxa-4,10,12-triazapentadecane-9,13,15-tricarboxylate (12)

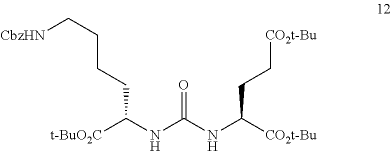

11(1.0 g, 3.38 mmol, 1.0 equiv.) and triethylamine (1.54 mL, 11.09 mmol, 3.28 equiv.) were dissolved in dichloromethane (30 mL) and cooled to −78° C. Triphosgene (341 mg, 1.15 mmol, 0.34 equiv.) in dichloromethane (10 mL) was added dropwise to the reaction mixture. Upon complete addition, the reaction was allowed to warm to room temperature and stirred for 30 minutes. 12 (757 mg, 2.03 mmol, 0.6 equiv) was added, followed by the addition of triethylamine (283 L, 2.03 mmol, 0.6 equiv.). The reaction was allowed to stir at room temperature overnight for 16 hours. The reaction was then diluted with dichloromethane (50 mL), and washed with water (100 mL×2). The crude mixture was dried over Na₂SO₄ and concentrated under reduced pressure. Column chromatography (Silica 1.5:1 hexane:ethyl acetate) yielded 4 (1.09 g, 86%) as a colorless oil with the following spectral characteristics: IR (thin film/KBr) 3342, 2976, 1731, 1650, 1552, 1454, 1368, 1255, and 1153 cm⁻¹; ¹H NMR (500 MHz, CDCl₃) δ 7.35 (d, J=3.75 Hz, 4H), 7.33-7.30 (m, 1H), 5.10 (d, J=4.55 Hz, 2H), 5.06-5.01 (m, 2H), 4.99 (s, 1H), 4.34-4.31 (m, 2H), 3.20-3.18 (m, 2H), 2.36-2.23 (m, 2H), 2.10-2.03 (m, 1H), 1.88-1.75 (m, 2H), 1.65-1.57 (m, 1H), 1.57-1.45 (m, 2H), 1.453 (s, 9H), 1.446 (s, 9H), 1.43 (s, 9H), 1.40-1.30 (m, 2H); ¹³C NMR (125 MHz, CDCl₃) δ□ 172.6, 172.5, 172.2, 136.8, 128.6, 128.5, 128.2, 82.2, 82.0, 80.7, 66.7, 53.4, 53.2, 40.7, 32.8, 31.7, 29.4, 28.5, 28.2, 28.1, 22.3; HRMS (EI+) m/z 622.3695 [calc'd for C₃₂H₅₁N₃O₉ (M+H)+ 622.3698].

### (S)-di-tert-butyl 2-(3-((S)-6-amino-1-tert-butoxy-1-oxohexan-2-yl)ureido)pentanedioate (12)

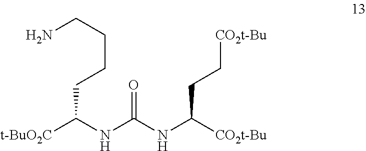

X (2.35 g, 3.78 mmol, 1.0 equiv.) was dissolved in methanol (37.8 ml) and was added dropwise to a vigorously stirred reaction flask containing dry 10% Pd/C (475 mg). H₂ was bubbled through the solution for 1-2 m, and then ran for 13 h under a balloon of H₂. The reaction was deemed complete by TLC (Rf=0.48 in 10% MeOH/CH₂Cl₂), plugged through celite, and concentrated to give a viscous oil, which was carried on without further purification.

### (S)-di-tert-butyl 2-(3-((S)-6-azido-1-tert-butoxy-1-oxohexan-2-yl)ureido)pentanedioate (14)

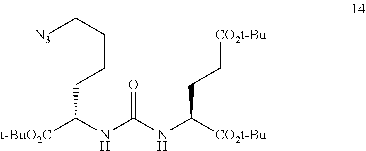

Sodium azide (2.629 g, 40.75 mmol, 10.0 equiv.) was dissolved in water (7.63 mL), and dichloromethane (12.91 mL) was added. The reaction mixture was cooled to 0° C. and triflic anhydride (1.36 mL, 8.09 mmol, 2.0 equiv.) was added. The solution was stirred for 3 h at rt, and the organic layer was separated from the aqueous layer. The aqueous layer was extracted with dichloromethane (3×4 mL). The organic layers were combined and washed with aqueous Na₂CO_{3(aq)} to give 25 ml of 0.391 M TfN₃. Amine 13 (1.97 g, 4.04 mmol, 1.0 equiv.) was dissolved in water (14.37 mL) and methanol (28.74). To this solution were added CuSO₄-5H₂O (10.1 mg, 0.04 mmol, 0.01 equiv.) and K₂CO₃ (837.5 mg, 6.06 mmol, 1.5 equiv.). The TfN₃ solution (25 ml, 8.09 mmol, 2 equiv.) was added rapidly to the stirring solution of 13, and the reaction stirred for 19 h at rt. The organic layer was separated from the aqueous layer, and the water/methanol layer was extracted once with dichloromethane. The combined organic layers were dried over MgSO4, concentrated under reduced pressure, and purified by column chromatography to yield 14 as a white solid (1.440 g, 71%). R_{f}=0.68 in 10% MeOH:CH₂Cl₂. IR (Thin film/NaCl) 3335, 2980, 2933, 2868, 2097, 1733, 1635, 1560, 1368, 1257, and 1155 cm⁻¹; ¹HNMR (500 MHz, CDCl₃) δ 5.01 (d, J=8.25 Hz, 2H), 4.34 (m, 2H), 3.26 (t, J=7.4 Hz, 2H), 2.35-2.25 (m, 2H), 2.09-2.05 (m, 1H), 1.87-1.76 (m, 2H), 1.66-1.55 (m, 3H), 1.46 (s, 18H), 1.43 (s, 9H), 1.45-1.35 (m, 2H) ppm; ¹³CNMR (125 MHz, CDCl₃) δ 172.6, 172.4, 172.2, 156.8, 82.3, 82.1, 80.7, 53.4, 53.2, 51.3, 33.0, 31.7, 28.6, 28.5, 28.2, 28.1, 22.4 ppm; HRMS (EI+) m z 514.3225 [calc'd for C₂₄H₄₃N₅O₇ (M+H)+514.3235].

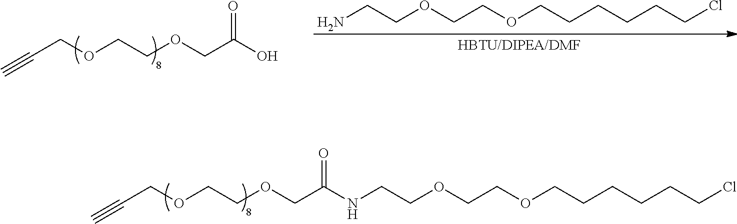

A propargyl containing intermediate, containing a halotag chloroalkane, a snaptag O6-benzyl guanine or O2-benzyl cytosine moiety is prepared by reacting an amine containing group with the carboxyl acid moiety of the propargyl acid in the presence of HBTU/DIPEA in DMF as solvent to provide the appropriately labeled proparyl intermediate which can be further condensed onto an azide to form a triazole connected compound according to the present invention.

The corresponding propargyl intermediate as prepared above or by analogy, is reacted with an azide (the t-butyl groups of compound 13 or 14 may be readily removed with TFA/DCM the compound with free carboxylic acid groups) as depicted below in the presence of CuSO4 and sodium ascorbate in aqueous solvent (e.g. water) to form triazole connected compounds according to the present invention. An example of such a reaction is presented below to provide a final bi-functional molecule (n is from 0-20, 1-15 or as otherwise disclosed herein).

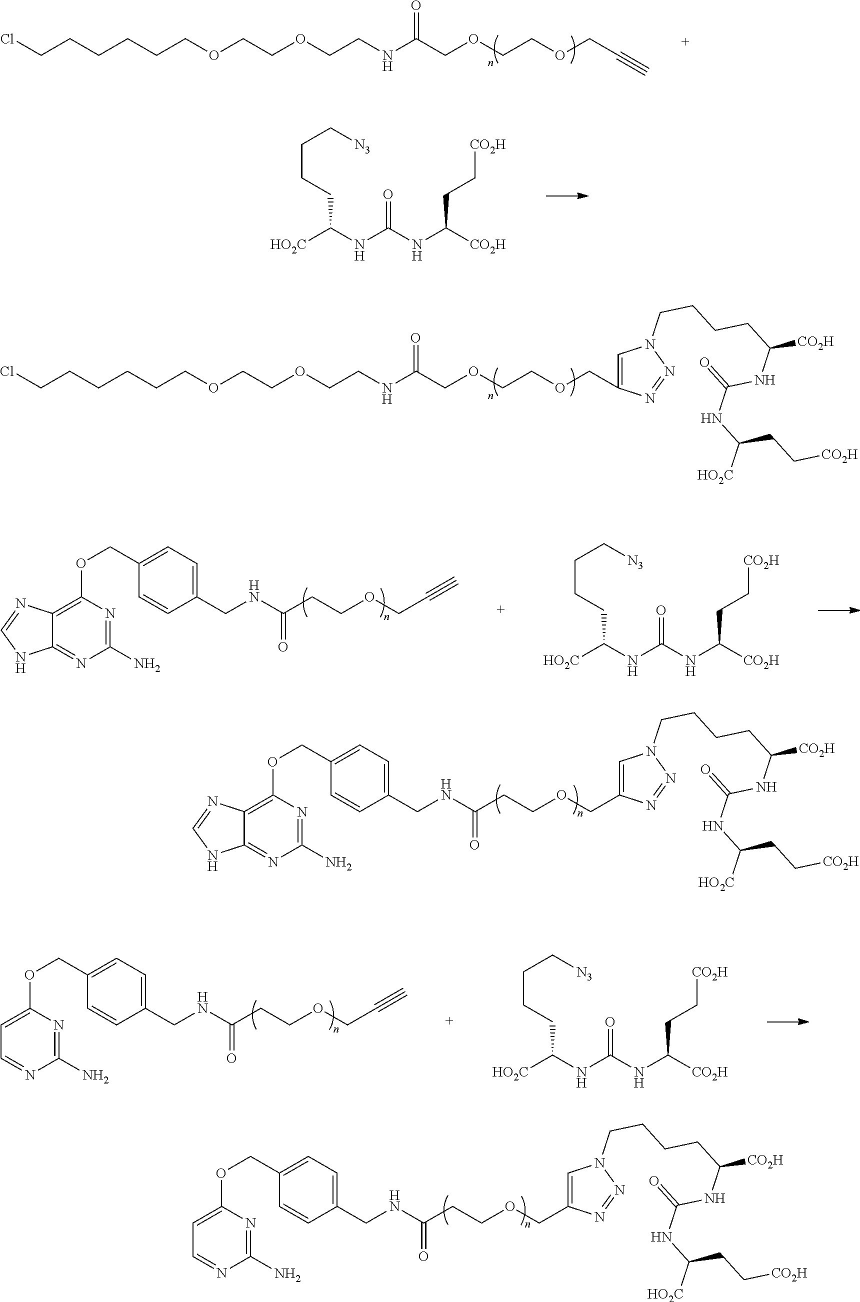

In the case of additional compounds, these are synthesized using the following chemical steps, or analogous steps.

In a first step, the FKBP binding moiety depicted below is converted to the corresponding carboxylic acid intermediate in the presence of TFA and DCM.

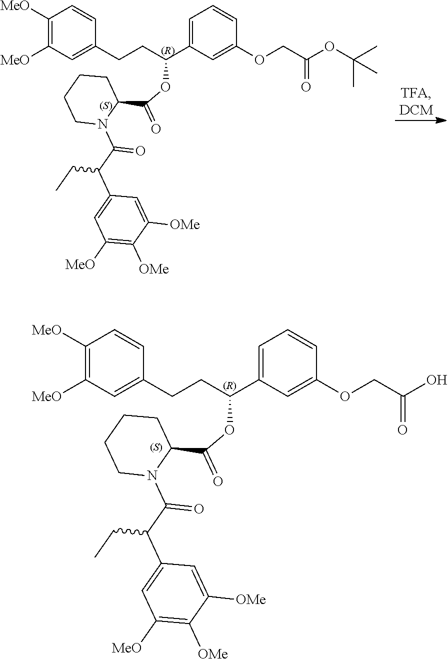

The carboxylic intermediate is then reacted with the propargyl PEG linked amine depicted below to provide the propargyl intermediate which can be condensed onto

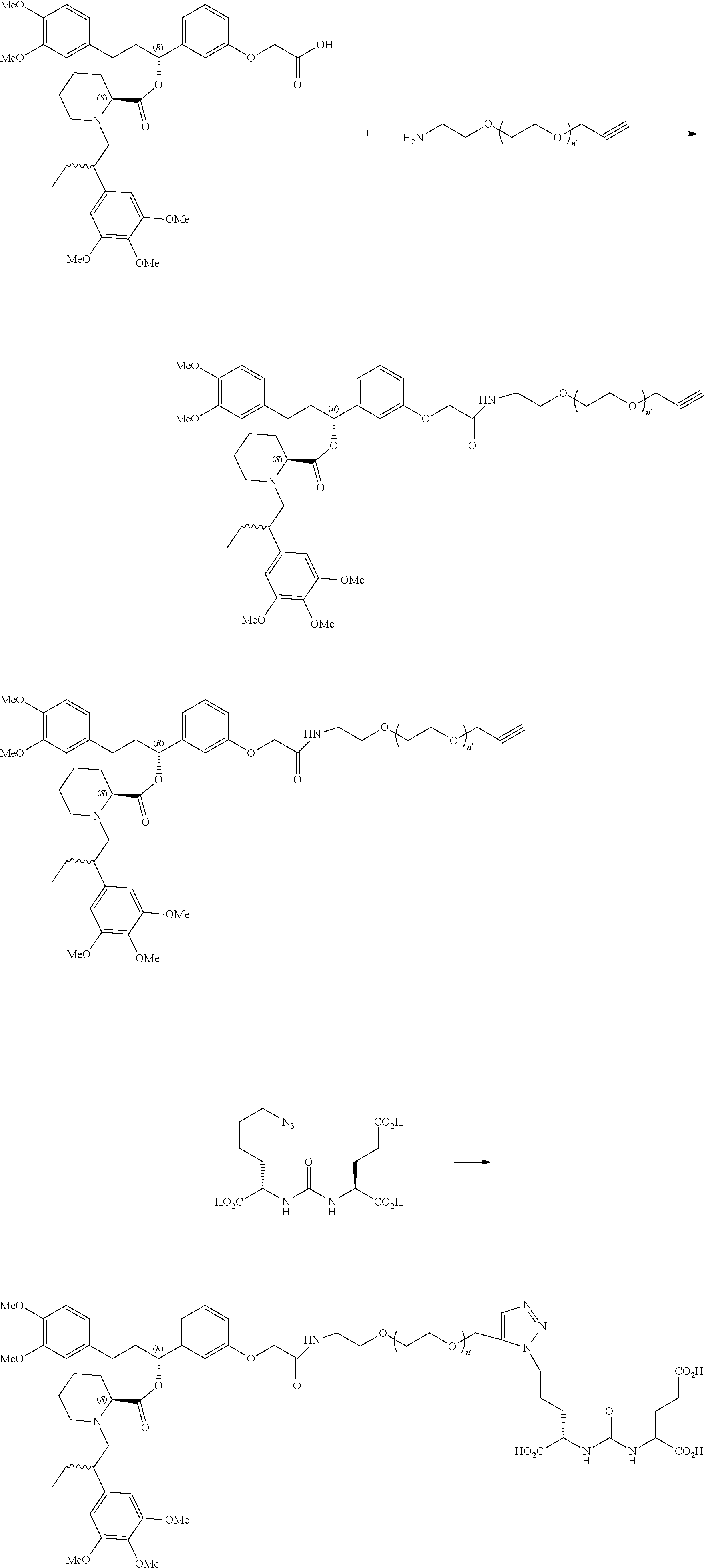

Other compounds according to the present invention are readily synthesized by analogy using the synthetic steps which are presented above. The moieties which are presented in FIG. 25 are readily coupled to provide bi-functional compounds according to the present invention.

### CAR T Cells

Sadelain, et al., The Basic Principles of Chimeric Antigen Receptor Design”, Cancer Discovery, April 2013 3; 388, describes fundamental principles of CAR T cell design and provides an overview of various techniques which can be employed to optimize a CAR T cell for particular clinical applications such as those described herein.

Further exemplary techniques for making useful CAR T cells are described in United States Patent Application Document No. 20150024482 as follows.

A “CAR can be designed to comprise a transmembrane domain that is fused to the extracellular domain of the CAR. [T]he transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. [Useful]transmembrane regions . . . may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively the transmembrane domain may be synthetic, in which case it will comprise predominantly hydrophobic residues such as leucine and valine.

The cytoplasmic domain or otherwise the intracellular signaling domain of the CAR . . . is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

[E]xamples of intracellular signaling domains . . . include the cytoplasmic sequences of the T cell receptor (TCR) and co-receptors that act in concert to initiate signal transduction following antigen receptor engagement, as well as any derivative or variant of these sequences and any synthetic sequence that has the same functional capability. It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences . . . include those derived from TCR zeta, FcR gamma, FcR beta, CD3 gamma, CD3 delta, CD3 epsilon, CD5, CD22, CD79a, CD79b, and CD66d.

[T]he cytoplasmic domain of the CAR can be designed to comprise the CD3-zeta signaling domain by itself or combined with any other desired cytoplasmic domain(s) . . . For example, the cytoplasmic domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, combinations of the aforementioned and a ligand that specifically binds with CD83, and the like.”

The invention is illustrated further in the following non-limiting examples.

### EXAMPLES

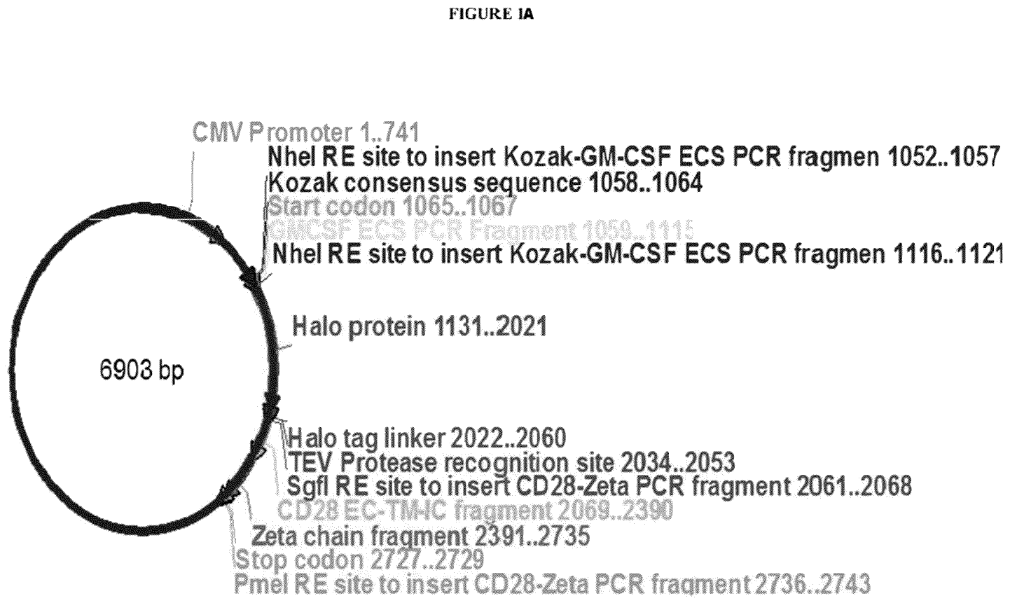

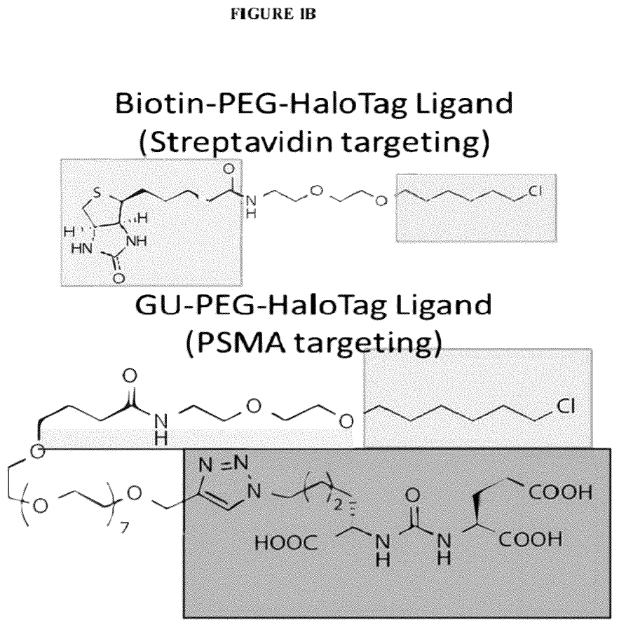

### Construction of Vectors—FIGS. 1A, 2 and 5

The following provides the details for the construction of vectors which are described or otherwise identified in FIGS. 1A, 2, 5. The DNA sequences of such vectors are presented in FIGS. 16-22 hereof. The methods are applicable to a wide variety of chimeric antigen receptors which can be used in the present invention.

### Lentiviral Production

For an overview on the lentiviral vector production, see, for example, Merten, et al., Molecular Therapy—Methods & Clinical Development (2016) 3, 16017. Typically, HEK 293 or HEK 293 T cells are used.

Although any method known in the art may be used to produce lentiviral vectors, in preferred embodiments of the present invention, lentivirus used in the present invention are produced using the Takara Bio pLVX lentiviral vector system (Takara Bio, 631988) in Takara Bio Lenti-X 293T packaging cells (Takara Bio, 632180). All lentivirus was produced following the Takara Bio lentiviral production protocol, with minor alterations: Lenti-X 293T cells (HEK293T cells) are passaged for example, in 90% Dulbecco's Modified Eagle's Medium (DMEM) with high glucose (4.5 g/L), 4 mM L-glutamine, and sodium bicarbonate (Sigma-Aldrich, D5796); 10% Tet-approved Fetal Bovine Serum (FBS) (Takara Bio, 631107); 100 units/ml penicillin G sodium, and 100 μg/ml streptomycin sulfate, 1 mM sodium pyruvate, and 1% non-essential amino acids (Thermo Fisher Scientific, 11140-050).

To produce virus, 4-5E6 Lenti—X 293T cells are plated on a 10-cm plate in 8 mL of media. When the plate reaches approximately 85-95% confluency (typically within 1-3 days), 7 ug of SMART-CAR pLVX plasmid as otherwise described herein, diluted into 600 μL of sterile water and mixed with one vial of Takara Bio packaging single-shots (Takra Bio, 631276). After 10 minutes of incubation at room temperature, the mixture is added dropwise to the Lenti—X plate and the plate is swirled. After an overnight incubation, 4 additional mL of media may be added to the plate. 48 hours after dropwise addition to the plate, all supernatant is collected from the plate and spun down for 10 minutes at 500×g. The resulting clear supernatant is then aliquoted into 500 μL aliquots and frozen for future use, or used directly in transductions without freezing. Immediately after removing the supernatant from the Lenti—X plate, a fresh 8 mL of warmed media is added back to the plate. 72 hours after the initial dropwise addition, supernatant is collected, spun, and aliquoted again.

### SMART CAR T Cells

In a preferred embodiment according to the present invention, in order to provide SMART Car T cells according to the present invention, primary human PBMCs from a healthy donor are thawed, bead selected on CD3 to isolate T cells, and activated with αCD3 and αCD28 activation beads. After 24 hours, DEAE-Dextran and SMART-CAR-encoding lentiviral supernatant was added to the primary human T cells to transduce lentiviral vector into the T cells, resulting in ˜6-10% transduction efficiency. Several days later, the transduced cells are stained for SMART-CAR expression and sorted for positivity. The sorted SMART-CAR cells were cultured with IL-2, and given fresh media and IL-2 every 2 days. 13 days after sorting, some of the cells are taken and used for further experimentation and/or therapy. In embodiments, the bifunctional molecule is bound to the antigen binding region of the CAR preferably after the CAR is introduced into the T cell and expressed such that the antigen binding region, exposed at the surface of the T cell, may be conjugated with the bi-functional molecule, depending on the nature of the antigen binding region as a halotag, snaptag or cliptag or alternatively, as a FKBP binding protein as otherwise described herein.

In preferred embodiments, the SMART CAR (T) cells of the present invention (i.e., CAR T cells which are conjugated with bi-functional agent are conjugated after the CAR T cells are produced. In preferred methods, the SMART-CAR expressing cells and the bifunctional molecule get mixed together at the same time or shortly before the commencement of therapy, although conjugated SMART-CAR T cells may be produced and formulated prior to the therapy. Although pre-manufacture and pre-incubation of CAR T cells and bi-functional molecules may be used prior to cancer therapy (e.g. for 10-15 minutes up to an hour or 2 prior to use), where the bifunctional molecule is added to the SMART-CAR expressing cells for a period prior to adding target cells, and then washing away excess before therapy begins to avoid a prozone/hook effect, the simultaneous addition of everything at the time of administration is also effective.

The following examples are provided to further describe the present invention.

SMART-CAR Polypeptide Construction/Cloning: CAR1, CAR2, CAR3, CAR4, CAR7, CAR10 and CAR13 of FIG. 5 Hereof-Individual Component Sequences are Presented in FIG. 24 Hereof. CAR Sequences are Set Forth in FIG. 23 and Vectors which Express the Various CAR Polypeptides are Presented in FIGS. 16-22 Hereof.

### CAR1 Construction:

Halotag pFN28A Vector
promega—catalog #G8441
   pLVX Vector

pLVX EF1alpha Puromycin vector, catalog #631988 from Takarra Bio

### GMCSF Component

SEQ ID NO: 45
Nucleotides 33-83 in NCBI M11220.1
Forward primer:
SEQ ID NO: 46
GTACTGCTAGCATGTGGCTGCAGAGCCTGC
Reverse primer:
SEQ ID NO: 47
TGGGTGCTAGCAGAGATGCTGCAGGCCACA

The forward primer contains an NheI restriction site, and a kozak sequence. The reverse primer also contains an NheI site.

### CD28 Component

Amino acids 114-220
SEQ ID NO: 48
Nucleotides 561-882 in NCBI NM_006139.3
Forward primer:
SEQ ID NO: 49
ACTGACGATCGGGAATTGAAGTTATGTATC
Reverse primer:
(SEQ ID NO: 50)
TGCGCTCCTGCTGAACTTCACTCTGGAGCGATAGGCTGCGAAGTCGCG

The reverse primer has sequence overlap with the CD3 zeta forward primer. The forward primer has an AsiSI restriction site.

### CD3 Zeta Component

(SEQ ID NO: 51)
Nucleotides 299-643 in NM_000734.3
Forward primer:
(SEQ ID NO: 52)
AGAGTGAAGTTCAGCAGGAGCGCA
Reverse primer:
(SEQ ID NO: 53)
CCTACGGTACCTCATGGCTGTTAGCGAGGGGGCAGGGCC

The reverse primer has a PmeI restriction site.

### CAR1 Construction Process:

To get the desired CD28 and CD3 Zeta sequences for use in this SMART-CAR, RNA was extracted from Jurkat T cells and reverse transcribed into cDNA. Then the noted primers were used to amplify the desired CD28, CD3 Zeta, and GMCSF sequences out from the Jurkat DNA. The CD3 Zeta and CD28 primers contained overlapping regions, allowing for a followup PCR to combine them into one contiguous sequence using the overlap PCR method. After combining, the CD28-CD3 Zeta insert was digested with AsiSI and PmeI. The pfN28A vector from Promega containing the Halotag sequence was also digested with AsiSI and PmeI. The Halotag pfN28A vector and CD28-CD3Zeta insert were ligated together and transformed. Resulting colonies with the correct insert were confirmed by sequencing. One of these new vectors containing Halotag, CD28, and CD3 Zeta were then digested with NheI, while also digesting the GMCSF insert with NheI. The Halotag-CD28-Zeta vector and the GMCSF insert were then ligated together and transformed. Colonies were screened until one with the correct GMCSF orientation was found, producing the full CAR1 construct (GMCSF-Halotag-CD28-CD3Zeta) in the pfN28A vector. At at later time, the CAR-1 construct was transferred into the pLVX vector backbone using conventional cloning techniques.

### CAR2 Construction

FKBP12 primers
Forward primer, with overlap of GMCSF-
(SEQ ID NO: 70)
CTGTGGCCTGCAGCATCTCTggagtgcaggtggaaaccatct
Reverse primer, with AsiSI restriction site-
(SEQ ID NO: 71)
actggaatctggcggtggatccGCGATCGCactga

### CAR2 Construction Process:

A plasmid containing FKBP12 F36V was ordered from Addgene. The above noted primers were used to amplify out the FKBP12 protein from the addgene vector. The GMCSF primers noted above for the cloning of CAR1 were used to amplify the desired GMCSF sequence. The FKBP12 F36V and GMCSF products were then combined in an overlap PCR amplification step. The resulting product was double digested with NheI and AsiSI, and inserted into a pFN28A CAR1 vector digested with the same restriction enzymes, yielding pfN28A CAR2. CAR2 was later moved into the pLVX vector using conventional cloning techniques.

### CAR3 and CAR4 Construction

CAR3 and CAR4 Cloning
CAR3 and CAR4 Primers
CD28 Forward (primer A)-
(SEQ ID NO: 72)
AGGGCCCACCCGCAAGCATTACCAGCCCTA
CD28 Reverse (with 41BB overlap) (primer B)-
(SEQ ID NO: 73)
AgtttctttctgccccgtttGGAGCGATAGGCTGCGAAGT
4-1BB Forward (primer C)-
(SEQ ID NO: 74)
aaacggggcagaaagaaactcctg
4-1BB Reverse (primer D)-
(SEQ ID NO: 75)
cagttcacatcctccttcttcttc
Zeta Forward (with 41BB overlap) (primer E)-
(SEQ ID NO: 76)
aagaaaggaggatgtgaactgAGAGTGAAGTTCAGCAGGAG
Zeta Reverse (primer F)-
(SEQ ID NO: 77)
ATTGAGCTCGTTATAGAGCTGGTT

### CAR 3 and CAR4 Construction Process

In order to clone CARs 3 and 4, RNA was extracted from jurkat T cells and reverse transcribed as described in cloning CAR1. 4-1BB primers C and D were used to amplify 4-1BB from the resulting cDNA. Primers A and B, and separately primers E and F, were used to PCR amplify CD28 and CD3 Zeta respectively from the pLVX CAR1 vector. The AB and EF amplification products both contained overlapping sections with the 4-1BB sequence. The AB product contained an ApaI restriction site, and the EF product contained a SacI restriction site. The AB product was then combined with the CD product using primers A and D to create a CD28-4-1BB insert via overlapping PCR amplification. This AD product was then combined with the EF product using primers A and F to create a CD28-41BB-CD3Zeta insert via overlapping PCR amplification. This resulting AF product was then digested with ApaI and SacI, and ligated into both the pLVX CAR1 and pLVX CAR2 vectors, both likewise digested with ApaI and Sac, creating pLVX CAR3 and pLVX CAR4 respectively.

### CAR7 Construction

CAR7 cloning

The object was to amplify the 335 a.a. region of human EGFR, specifically residues 310 to 644 in RCSB PDB structure 1YY9.

EGFRt Forward primer (primer A)-
(SEQ ID NO: 78)
CTGTGGCCTGCAGCATCTCTcgcaaagtgtgtaacggaataggtatt
EGFRt Reverse primer (primer B)-
(SEQ ID NO: 79
ggttgattgttccagacgcgTTAcacatgaagaggccgatccc
Zeta Forward primer (primer C)-
(SEQ ID NO: 80)
AAGAACCCTCAGGAAGGCCTGTACAATGAACTGCAGAAAG
Zeta Reverse Primer (primer D)-
(SEQ ID NO: 81)
aagttagtagctccgcttccGCGAGGGGGCAGGGC
P2A forward (primer E)-
(SEQ ID NO: 82)
ggaagcggagctact
GMCSF Reverse (primer F)-
(SEQ ID NO: 83)
AGAGATGCTGCAGGC
Zeta forward (primer G)-
(SEQ ID NO: 84)
CAGGAAGGCCTGTACaatga
EGFRt reverse (primer H)-
(SEQ ID NO: 85)
cagttcctgtggatccagag

### IDT Fragment:

(SEQ ID NO: 86)
CAGGAAGGCCTGTACAATGAACTGCAGAAAGATAAGATGGCGGAG
GCCTACAGTGAGATTGGGATGAAAGGCGAGCGCCGGAGGGGCAAG
GGGCACGATGGCCTTTACCAGGGTCTCAGTACAGCCACCAAGGAC
ACCTACGACGCCCTTCACATGCAGGCCCTGCCCCCTCGCGGAAGC
GGAGCTACTAACTTCAGCCTGCTGAAGCAGGCTGGAGACGTGGAG
GAGAACCCTGGACCTATGTGGCTGCAGAGCCTGCTGCTCTTGGGC
ACTGTGGCCTGCAGCATCTCTCGCAAAGTGTGTAACGGAATAGGT
ATTGGTGAATTTAAAGACTCACTCTCCATAAATGCTACGAATATT
AAACACTTCAAAAACTGCACCTCCATCAGTGGCGATCTCCACATC
CTGCCGGTGGCATTTAGGGGTGACTCCTTCACACATACTCCTCCT
CTGGATCCACAGGAACTG

### CAR7 Construction Process

To make CAR7, RNA was extracted from LNCaP cells and reverse transcribed into cDNA. Primers A and B were used to amplify the desired EGFRt sequence from the cDNA, making product AB. Primers C and D were used to amplify the desired insert from pLVX CAR3, yielding product CD. In order to generate the P2 Å and GMCSF portions of CAR7, the above IDT DNA fragment was ordered from Integrated DNA Technologies. Primers E and F were used to amplify the desired insert out from the IDT fragment, yielding product EF. Products AB, CD, and EF were all combined in a single NEB HiFi DNA Assembly reaction, yielding the full desired EGFRt insert, consisting of:

(SEQ ID NO: 87)
AAGAACCCTCAGGAAGGCCTGTACAATGAACTGCAGAAAGATAAG
ATGGCGGAGGCCTACAGTGAGATTGGGATGAAAGGCGAGCGCCGG
AGGGGCAAGGGGCACGATGGCCTTTACCAGGGTCTCAGTACAGCC
ACCAAGGACACCTACGACGCCCTTCACATGCAGGCCCTGCCCCCT
CGCGGAAGCGGAGCTACTAACTTCAGCCTGCTGAAGCAGGCTGGA
GACGTGGAGGAGAACCCTGGACCTATGTGGCTGCAGAGCCTGCTG
CTCTTGGGCACTGTGGCCTGCAGCATCTCTCGCAAAGTGTGTAAC
GGAATAGGTATTGGTGAATTTAAAGACTCACTCTCCATAAATGCT
ACGAATATTAAACACTTCAAAAACTGCACCTCCATCAGTGGCGAT
CTCCACATCCTGCCGGTGGCATTTAGGGGTGACTCCTTCACACAT
ACTCCTCCTCTGGATCCACAGGAACTGGATATTCTGAAAACCGTA
AAGGAAATCACAGGGTTTTTGCTGATTCAGGCTTGGCCTGAAAAC
AGGACGGACCTCCATGCCTTTGAGAACCTAGAAATCATACGCGGC
AGGACCAAGCAACATGGTCAGTTTTCTCTTGCAGTCGTCAGCCTG
AACATAACATCCTTGGGATTACGCTCCCTCAAGGAGATAAGTGAT
GGAGATGTGATAATTTCAGGAAACAAAAATTTGTGCTATGCAAAT
ACAATAAACTGGAAAAAACTGTTTGGGACCTCCGGTCAGAAAACC
AAAATTATAAGCAACAGAGGTGAAAACAGCTGCAAGGCCACAGGC
CAGGTCTGCCATGCCTTGTGCTCCCCCGAGGGCTGCTGGGGCCCG
GAGCCCAGGGACTGCGTCTCTTGCCGGAATGTCAGCCGAGGCAGG
GAATGCGTGGACAAGTGCAACCTTCTGGAGGGTGAGCCAAGGGAG
TTTGTGGAGAACTCTGAGTGCATACAGTGCCACCCAGAGTGCCTG
CCTCAGGCCATGAACATCACCTGCACAGGACGGGGACCAGACAAC
TGTATCCAGTGTGCCCACTACATTGACGGCCCCCACTGCGTCAAG
ACCTGCCCGGCAGGAGTCATGGGAGAAAACAACACCCTGGTCTGG
AAGTACGCAGACGCCGGCCATGTGTGCCACCTGTGCCATCCAAAC
TGCACCTACGGATGCACTGGGCCAGGTCTTGAAGGCTGTCCAACG
AATGGGCCTAAGATCCCGTCCATCGCCACTGGGATGGTGGGGGCC
CTCCTCTTGCTGCTGGTGGTGGCCCTGGGGATCGGCCTCTTCATG
TAACGCGTCTGCTGGAACAATCAACC

This resulting insert was then digested with BsRGI and MluI, and ligated into the pLVX CAR3 vector digested with the same restriction enzymes. A resulting colony was selected and found to contain two undesired mutations in the GMCSF and P2A regions (pLVX CAR7 mutant). To correct this, primers G and H were used to amplify the noted IDT fragment. The resulting GH PCR product was digested with BsrGI and BamHI, and ligated into the pLVX CAR7 mutant vector digested with the same restriction enzymes, producing the full pLVX CAR7 construct with no mutations present.

### CAR10 Construction

### CAR10 Cloning

### IDT CAR10 Insert:

(SEQ ID NO: 88)
GGTGAATTCGTTAACCATATGTTAATTAACGCCACCATGTGGCTG
CAGAGCCTGCTGCTCTTGGGCACTGTGGCCTGCAGCATCTCTATG
GACAAAGACTGCGAAATGAAGCGCACCACCCTGGATAGCCCTCTG
GGCAAGCTGGAACTGTCTGGGTGCGAACAGGGCCTGCACCGTATC
ATCTTCCTGGGCAAAGGAACATCTGCCGCCGACGCCGTGGAAGTG
CCTGCCCCAGCCGCCGTGCTGGGCGGACCAGAGCCACTGATGCAG
GCCACCGCCTGGCTCAACGCCTACTTTCACCAGCCTGAGGCCATC
GAGGAGTTCCCTGTGCCAGCCCTGCACCACCCAGTGTTCCAGCAG
GAGAGCTTTACCCGCCAGGTGCTGTGGAAACTGCTGAAAGTGGTG
AAGTTCGGAGAGGTCATCAGCTACAGCCACCTGGCCGCCCTGGCC
GGCAATCCCGCCGCCACCGCCGCCGTGAAAACCGCCCTGAGCGGA
AATCCCGTGCCCATTCTGATCCCCTGCCACCGGGTGGTGCAGGGC
GACCTGGACGTGGGGGGCTACGAGGGCGGGCTCGCCGTGAAAGAG
TGGCTGCTGGCCCACGAGGGCCACAGACTGGGCAAGCCTGGGCTG
GGTGAGCCAACCACTGAGGATCTGTACTTTCAGAGCGATAACGCG
ATCGCAATTGAAGTTATGTATCCTCCTCCTTACCTAGACAATGAG
AAGAGCAATGGAACCATTATCCATGTGAAAGGGAAACACCTTTGT
CCAAGTCCCCTATTTCCCGGACCTTCTAAGCCCTTTTGGGTGCTG
GTGGTGGTTGGTGGAGTCCTGGCTTGCTATAGCTTGCTAGTAACA
GTGGCCTTTATTATTTTCTGGGTGAGGAGTAAGAGGAGCAGGCTC
CTGCACAGTGACTACATGAACATGACTCCCCGCCGCCCCGGGCCC
ACCCGCAAGCATTACCAGCCCTATGCCCCACCACGCGACTTCGCA
GCCTATCGCTCCAAACGGGGCAGAAAGAAACTCCTGTATATATTC
AAACAACCATTTATGAGACCAGTACAAACTACTCAAGAGGAAGAT
GGCTGTAGCTGCCGATTTCCAGAAGAAGAAGAAGGAGGATGTGAA
CTGAGAGTGAAGTTCAGCAGGAGCGCAGACGCCCCCGCGTACCAG
CAGGGCCAGAACCAGCTCTATAACGAGCTCAATCTAGGACGAAGA
GAGGAGTACGATGTTTTGGACAAGAGACGTGGCCGGGACCCTGAG
ATGGGGGGAAAGCCGAGAAGGAAGAACCCTCAGGAAGGCCTGTAC
AATGAACTGCAGAAAGATAAGATGGCGGAGGCCTACAGTGAGATT
GGGATGAAAGGCGAGCGCCGGAGGGGCAAGGGGCACGATGGCCTT
TACCAGGGTCTCAGTACAGCCACCAAGGACACCTACGACGCCCTT
CACATGCAGGCCCTGCCCCCTCGCTAACAGCCAactagtGTTTAA
ACGAATTCGGGCTCGGTACCCGGGGATCCTCTAGAGTC

The above DNA fragment containing the sequence for the SNAP-TAG protein, along with a kozak sequence and a GMCSF signal sequence, was ordered from Integrated DNA Technologies. It was double digested directly with EcoRI and Spel, and ligated into an empty pLVX vector backbone (catalog #631988 from Takarra Bio) digested with the same restriction enzymes. Following transformation, sequencing confirmed the final desired sequence.

### CAR13 Construction

### CAR13 Cloning

To generate CAR13, pLVX CAR10 and pLVX CAR7, prepared above, were both digested with EcoRI and AsiSI. The resulting insert from pLVX CAR10 and the vector from pLVX CAR7 were purified and then ligated together to produce pLVX CAR13.

### Example 1

### First Generation Smart-CAR Construct

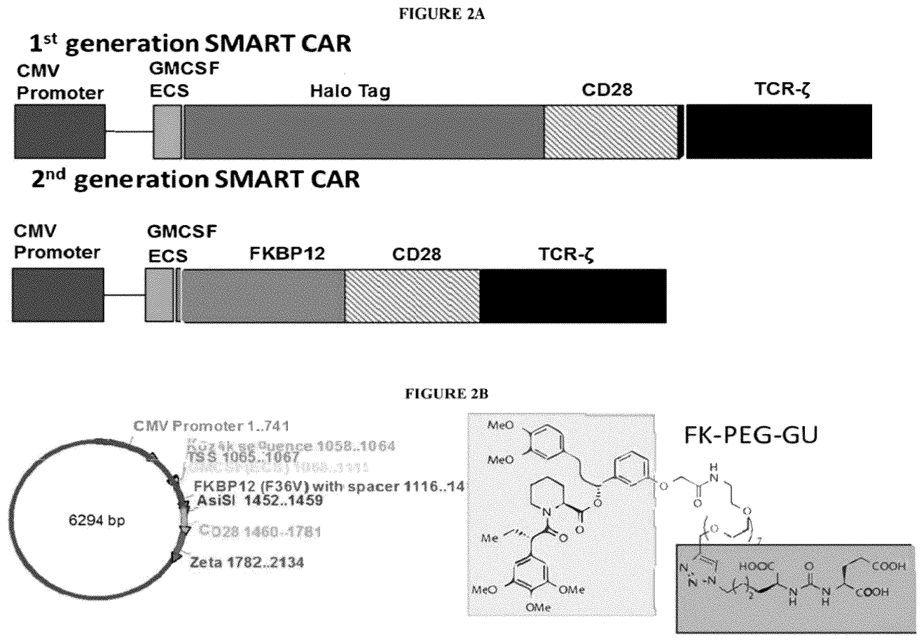

In order to demonstrate proof of principle, the inventors designed a first generation of synthetic SMART chimeric antigen receptor fusion protein consisting of a GMCSF extra cellular signal sequence (ECS), Halotag enzyme (Promega), Transmembrane and minimal signaling domain of CD28 and the Zeta signaling domain of the T-Cell receptor (FIGS. 1A and 2) and provided constructs as described above.

### Example 2

### Second Generation SMART-CAR Construct

A second and subsequent generations of SMART-CAR have been cloned and evaluated for surface expression in the case of the second Generation CAR replacing the bulky HaloTag protein with a smaller mutant FKBP12 protein which binds with very high specificity and affinity to its cognate F506 based ligand designed to target PSMA expressed on the surface of prostate cancer cells (FIG. 2). Later generations of CAR polypeptides, e.g., CAR3, CAR4, CAR7, CAR10 and CAR13, described above and in FIG. 5, contain slightly different components in order to promote and examine further efficiencies in expression and in targeting cancer cells in combination with conjugated bi-functional compounds as described herein.

### Example 3

### Evaluation of the SMART-CARs In-Vitro: Surface Expression of the Engineered SMART-CAR in Jurkat T-Cells

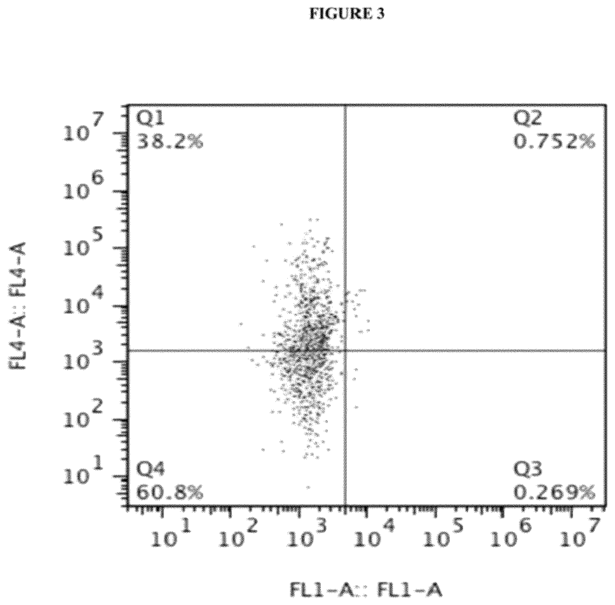

The first generation SMART-CAR construct was transfected into the CD4+ Jurkat T-cells by electroporation using a Nucleofection device. Appropriate targeting of the fusion protein to the cell surface by the GMCSF extracellular signaling domain was verified using a Halo ligand conjugated to AF660 fluorophore or commercially available anti Halo protein specific mouse monoclonal antibodies (FIG. 3).

### Example 4

### T-Cell Like Signaling by the Engineered SMART-CARs in Jurkat T-Cells

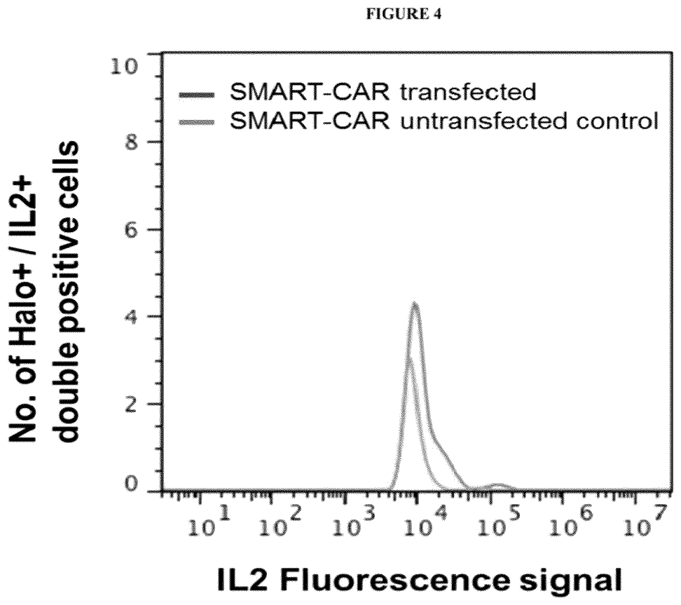

Stimulation of the T-Cell receptor of the Jurkat cells leads to production of IL2 in Jurkat cells. The ability of the engineered SMART-CAR to transduce T-cell like signaling and effector activity was evaluated by IL2 cytokine production upon stimulation of the Halo CAR construct using a small molecule adapter (FIG. 1) directed against Streptavidin. Streptavidin which functioned as a target of the engineered T-Cells was immobilized either on cell sized beads or on surface of microtiter plates. In the transfected cells, a significantly increased IL-2 production and accumulation was detectable by flow cytometry in presence of a Biotin-HaloTag small molecule adapter (FIG. 4).

### Example 5

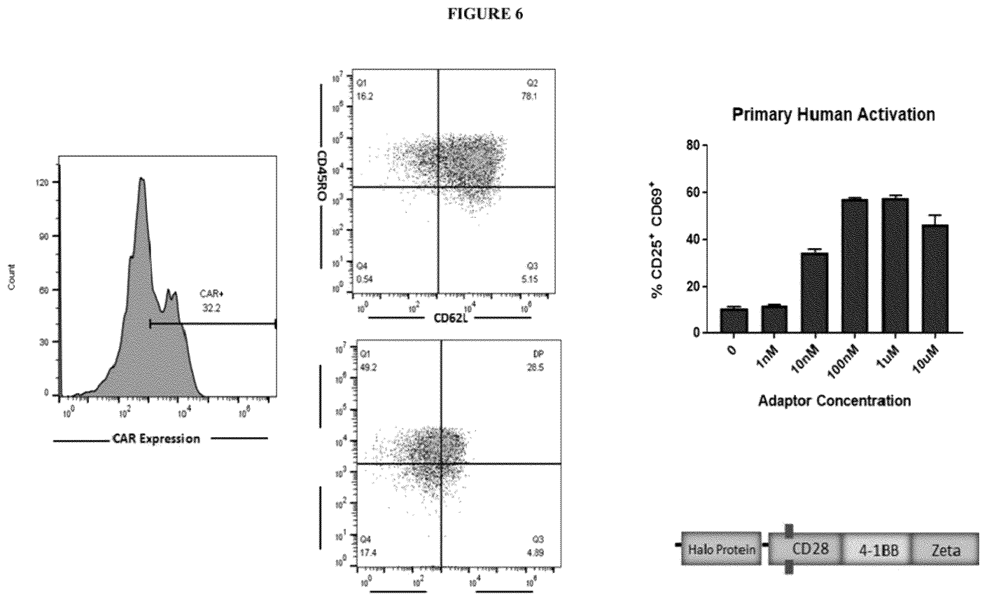

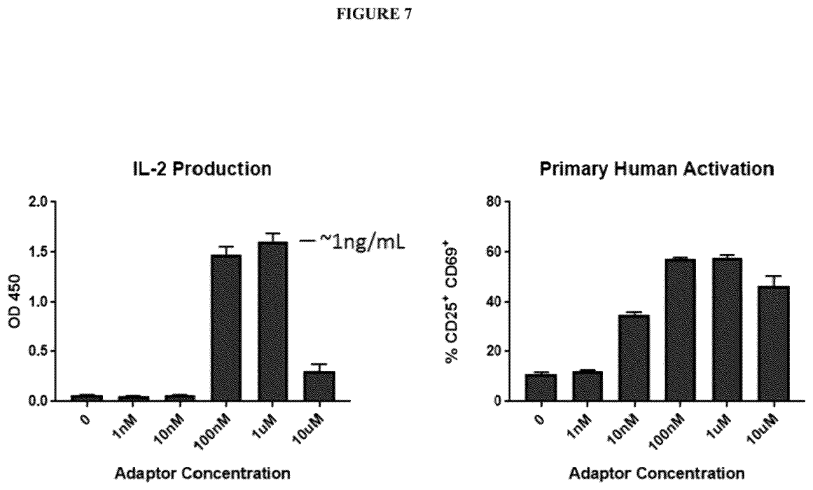

Experiments evidenced that SMART-CAR induced activation and cytokine secretion in primary human T cells. Cells cultured as mentioned above were taken and used for activation studies. 5E4 CAR3⁺ cells were co-incubated with κE4 LNCaP cells with various PSMA-specific SMART-CAR adaptor concentrations as indicated at 37° C. for 24 hours in 96-well round bottom plates. After 24 hours, 50 uL of supernatant per condition were taken to run an TL-2 cytokine ELISA, and the cells were separately taken and stained for CD69 and CD25 activation markers via Flow cytometery (see FIGS. 6 and 7). SMART-CAR expression at the time of assay was found to be approximately 32%. The staining for TL-7 and PD-1 seen in FIG. 6 was done at the time of assay analysis, while the staining for CD62L and CD45RO (showing a predominantly memory phenotype) was done on the cultured cells one day prior to the assay analysis (at assay start time). All samples for the activation markers and ELISA in FIGS. 6-7 were run in triplicate. The y-axis of the cytokine production chart is OD due to an issue with the TL-2 standard for that experiment. The estimate of ˜1 ng/mL peak production is based on a previous ELISA run with a nearly identical outcome, which had a working standard for quantitation. The ˜1 ng/mL should only be taken as a very rough estimate due to the poor comparability of OD values between ELISAS when lacking a standard for comparison.

### Example 6

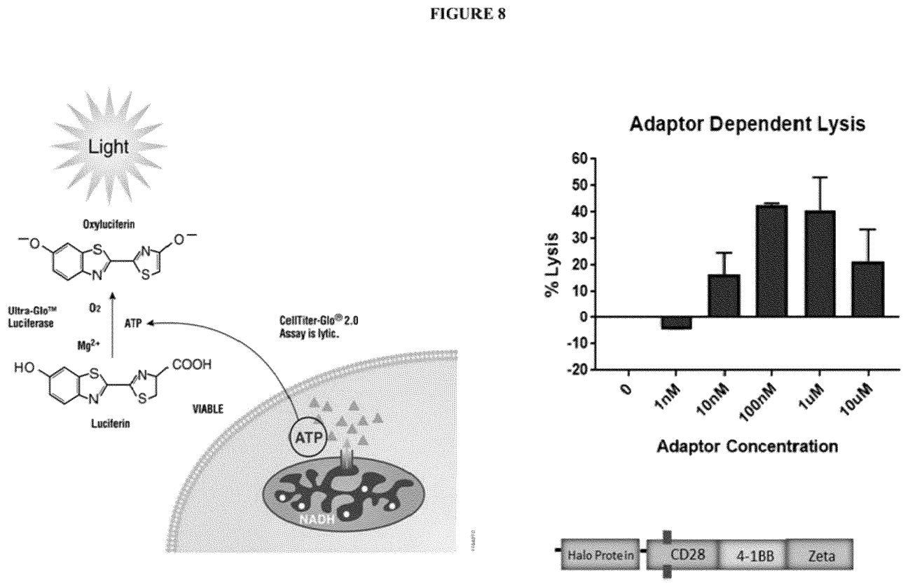

This experiment was designed to test the toxicity of SMART CAR (T) cells against LNCaP Cells. The dose-dependent cytotoxicity of SMART-CAR primary human T cells against LNCaP cells was tested. A celltiterglo cytotoxicity assay was used to assay the ability of SMART-CARs to induce cytotoxicity against LNCaP tumor cells in primary human T cells. 1E4 LNCaP cells were detached with an EDTA detachment solution and added to wells of a 96-well plate. Approximately 1E4 CAR3⁺ primary human T cells, cultured as described above, were added to each well, for an E:T ratio of 1:1. The CAR3 expression percentage was about 18% at the time of this assay, so many CAR3⁻ primary human T cells are present as bystanders. The primary T cell phenotypic makeup was approximately ˜63% CD8⁺ and ˜31% CD4⁺. Immediately after the LNCaP and T cells were combined, Halo-PSMA adaptor ligand was added as indicated on the slide. Wells were topped up to 100 uL with media, and the plate was incubated at 37° C. for 16 hours. At the end of 16 hours, a standard celltiterglo protocol was followed to analyze the wells. Briefly: the plate was equilibrated to room temperature for 45 minutes; celltiterglo solution was added to wells to lyse cells; plate was mixed for 5 minutes and then placed at rest for 5 minutes; wells were analyzed for luminescence on a plate reader. Some set aside primary human cells were analyzed by flow cytometry at the assay endpoint to give the above noted percentage characteristics. For % lysis calculation, 0% lysis was set to the luminescence signal for primary human T cells and LNCaP cells, with no ligand. This signal was very close to primary human cells alone+LNCaP cells alone, suggesting little to no background lysis. The small negative % lysis with 1 nM adaptor is thought to be noise. All samples were tested in duplicate. The results are presented in FIG. 8.

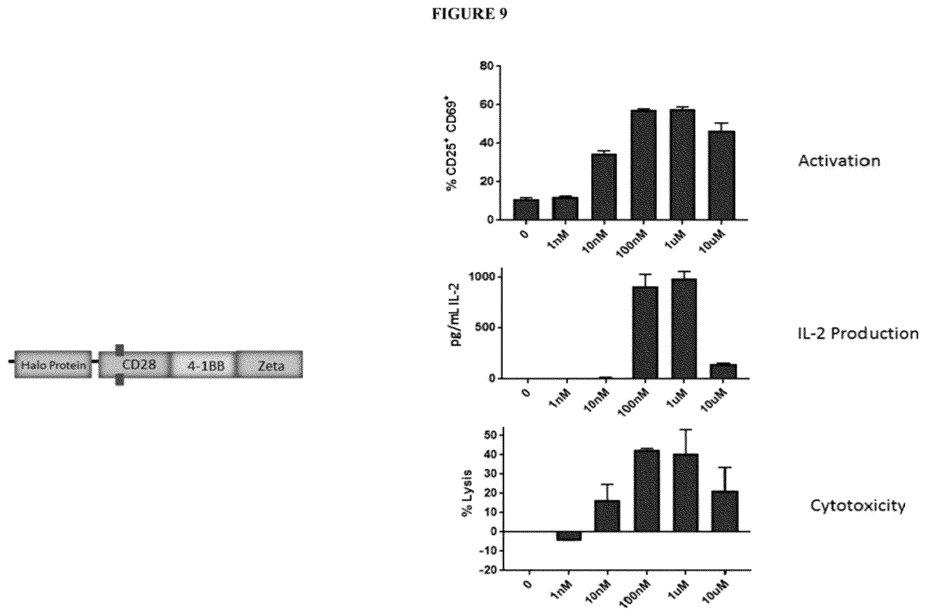

FIG. 9 shows a summary of SMART-CAR activity in primary human T cells. This slide shows the data from FIGS. 6-8 aligned in order to highlight the consistent activation pattern observed. Peak activation is observed at approximately the 100 nM-1 uM level of adaptor. The sharp fall-off in IL-2 production outside of 100 nM and 1 uM may have to do with the fact that CD25, a receptor for IL-2, is being upregulated during this assay (due to activation). Repeating the cytokine production assay in the presence of antagonistic αCD25 antibodies could potentially broaden the curve. Decreasing activation above 1 uM is likely due to the prozone effect, due to the three body binding dynamics of this system. When adaptor is pre-incubate and then washed away rather than left present for the duration, activation at higher concentrations remains near peak levels (previous data).

### Example 6

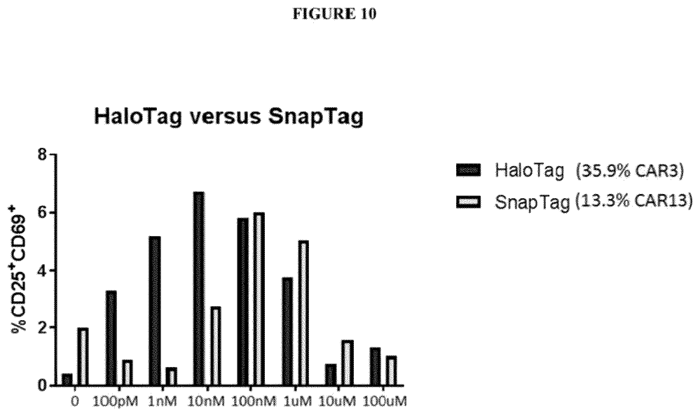

This experiment was conducted to determine and show the comparison of Halotag activation versus Snaptag activation of SMART-CAR T cells. Jurkat T cells expressing either a Halotag-based SMART-CAR or a Snaptag-based SMART CAR were incubated for 24 hours at 37° C. at 1:1 E:T ratio with LNCaP cells in 96 well round bottom plates in the presence of PSMA-specific SMART-CAR adaptor as indicated. After 24 hours, cells were taken and stained for activation markers. Flow cytometry results are plotted, showing a similar pattern of activation between the Snaptag and Halotag based SMART-CARs, although with the peak activity for Snaptag falling at a higher adaptor concentration. Expression plotted shows the SMART-CAR expression read out at time of assay analysis. Although total double positive percentages are low, these levels of activation are notable when considering the low overall expression levels of the constructs. 6% peak activation for Snaptag, for instance, could represent up to ˜46% activation (6/13) of the Snaptag-CAR-expressing cells. Due to a shortage of cells, this experiment included only one sample per condition. This data demonstrates that Snaptag can function as a SMART-CAR extracellular domain, similarly to Halotag. The results are presented in FIG. 10.

### Example 7

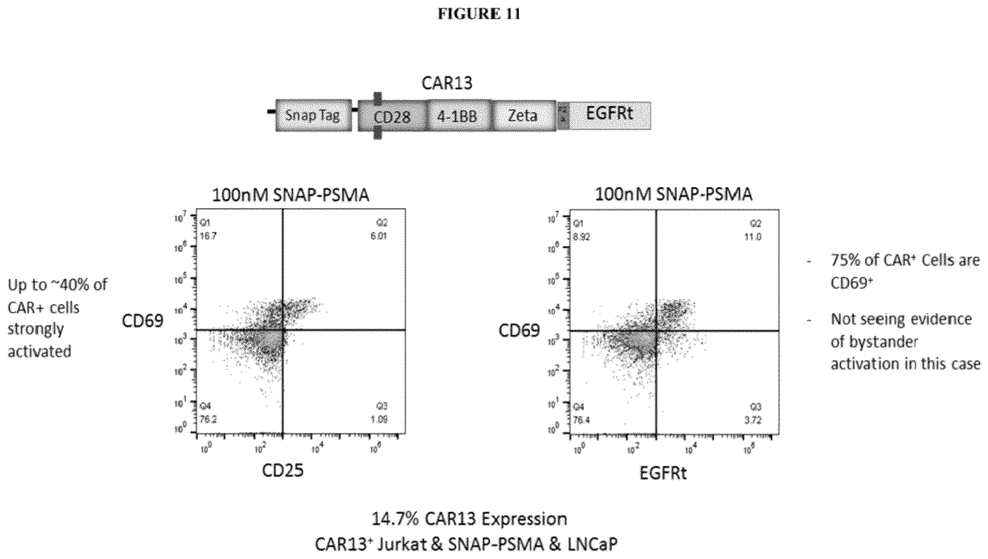

The experiment shows the use of EGFRt to track SMART-CAR specific activation. These plots are generated from the same experiment as described in Example 6, FIG. 10, above. Using EGFRt expression as a proxy for SMART-CAR expression, the right plot of FIG. 11 demonstrates that ˜75% (11/14.7) of C13 SMART-CAR⁺ Jurkats are activated to some degree in this assay. The right plot also demonstrates that nearly all above-background CD69⁺ cells are SMART-CAR⁺, suggesting little activation of bystander T cells in this assay. Looking at overall activation (right plot), up to ˜40% of the C13 SMART-CAR⁺ Jurkats may have been strongly activated (CD69⁺CD25⁺) in this assay.

### Example 8

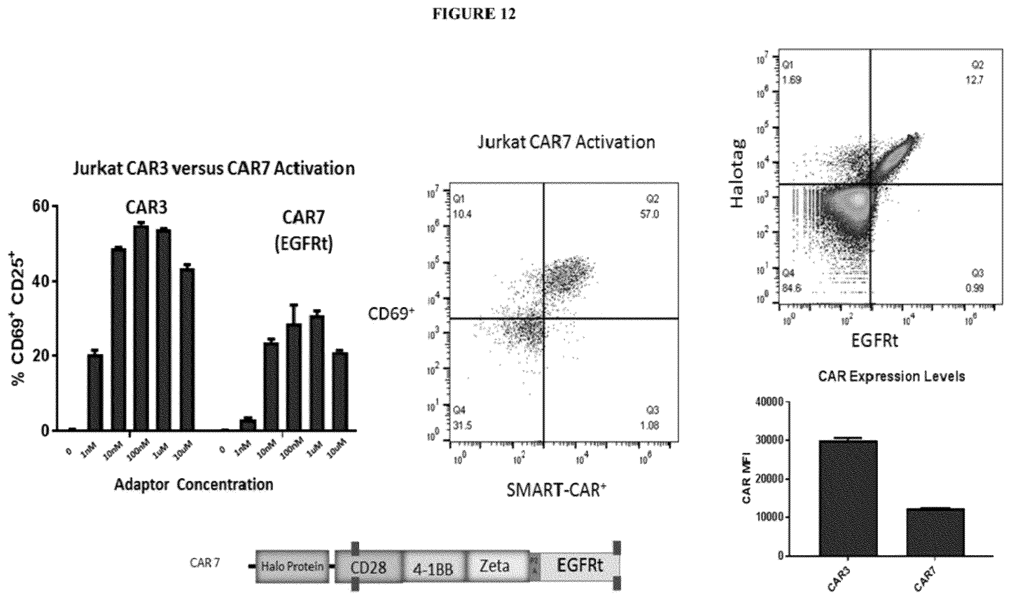

This is an experiment which compared activation of EGFRt-containing Halotag-based SMART-CAR to non-EGFRt SMART-CAR. 5E4 Jurkat T cells with 78% CAR3 (non-EGFRt) expression and 5E4 Jurkat T cells with 61% CAR7 (with EGFRt) expression were co-incubated with equal numbers of LNCaP cells at 37° C. for 24 hours in the presence of PSMA-specific SMART-CAR adaptor as indicated. After 24 hours, cells were taken and stained for flow cytometry analysis. Despite there being a relatively small difference in total expression, CAR7 (containing EGFRt) seemed to reach lower levels of overall activation compared to CAR3. The overall activation pattern remained the same (FIG. 12, left graph). A direct co-stain of Halotag and EGFRt (FIG. 12, top right) demonstrates that the two are expressed at nearly 1:1 in CAR7 cells, making EGFRt a good marker for CAR⁺ cells. When only looking at CD69 on the CAR7 cells (FIG. 12, center plot), it can be seen that nearly all CAR7T cells are CD69+(based on EGFRt expression), suggesting that all of the CAR⁺ cells are getting activated, but not strongly enough to match the high CD69⁺CD25⁺ double positive rates seen with CAR3. When both cell sources were stained with the same Halotag fluorophore, MFI revealed an apparent lower MFI for CAR7 (FIG. 12, lower right graph), suggesting a lower level of expression on the cell surface. This lower level of expression could explain the decreased magnitude of activation. All samples for the activation graph and MFI graph were performed in duplicate.

### Example 9

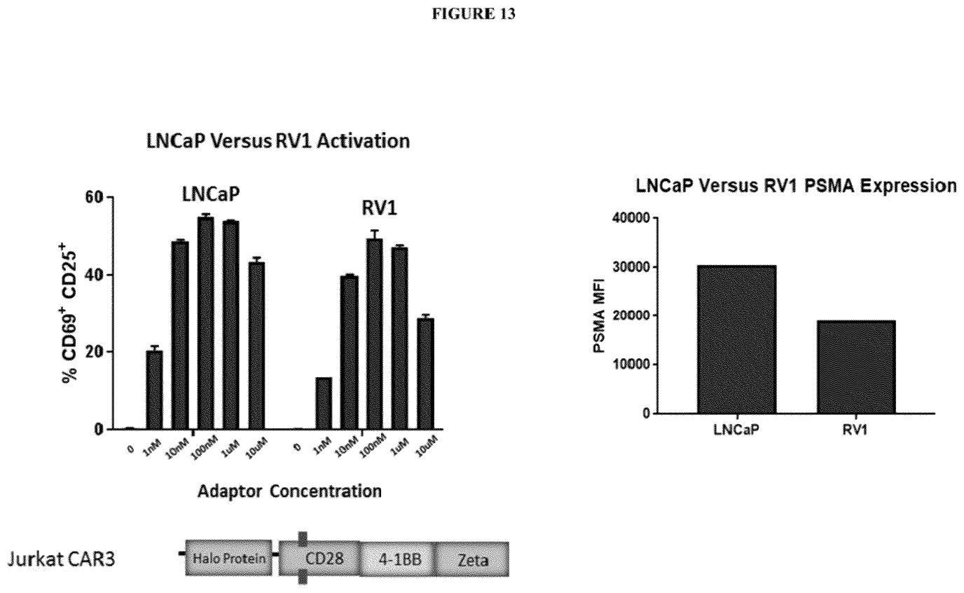

This experiment compared CAR3 activation against different PSMA⁺ cell lines. Included in the same experiment described above for FIG. 11, example 7 were additional wells containing RV1 cells, which were co-incubated with Jurkat CAR3 cells in the same manner as with LNCaP cells. The ‘LNCaP’ activation data on the left graph of FIG. 13 is the same as ‘CAR3’ from FIG. 12, being the same experiment. Plotted along with it is ‘RV1’ activation, showing Jurkat CAR3 cells co-incubated with RV1 cells in an identical manner, in the same assay. The similarity in pattern and magnitude of Jurkat activation reached between LNCaPs and RV1 suggests that the difference in PSMA expression (right plot) between LNCaP and RV1 cells does not significantly affect the responsiveness of SMART-CAR⁺ Jurkat cells.

### Example 10

This experiment determined bead selection of EGFRt⁺ SMART-CAR cells. Jurkat T cells expressing 7.8% CAR13 (which includes EGFRt) were positively selected using magnetic beads conjugated to αEGFRt antibodies. FIG. 14, left plot. The right plot of FIG. 14 demonstrates that significant enrichment can be achieved with the beads, providing an easy route for enrichment of SMART-CAR⁺ cells.

The above examples evidence that the SMART CAR T Cells exhibit biological activity which is consistent with their use as anticancer compounds and in therapy for the treatment of cancer.

1-63. (canceled)

64. A chimeric antigen receptor (CAR) T cell comprising a bi-functional molecule conjugated thereto, the chimeric antigen receptor (CAR) of the CAR T cell comprising an antigen binding domain, a hinge domain, a transmembrane domain, a co-stimulatory signaling region and a signaling domain, wherein the CAR antigen binding domain is not a prostate-specific membrane antigen (PSMA) domain and the bi-functional molecule is specific for both the antigen binding domain of the chimeric antigen receptor (CAR) T cell and prostate-specific membrane antigen (PSMA) wherein
(a) the antigen binding domain of the chimeric antigen receptor (CAR) T cell comprises a modified haloalkane dehydrogenase protein reactive with chloroalkane derivatives, a O6-methylguanine-methyltransferase (AGT) protein reactive with O6-benzylguanine derivatives or a AGT protein reactive with O2-benzylcytosine derivatives and said bi-functional molecule is conjugated to said CAR T cell through a C₃-C₁₀ haloalkane moiety when said antigen binding domain comprises said modified haloalkane dehalogenase protein, a O6 benzyl guanine moiety when said antigen binding domain comprises a AGT protein reactive with O6-benzylguanine derivatives and a O2-benzylcytosine moiety when said antigen binding domain comprises a AGT protein reactive with O2-benzylcytosine derivatives;
(b) the hinge domain comprises a hinge domain of CD28, 4-1BB, OX40, CD3-zeta, CD 8, CD-8 alpha, T cell receptor α or β chain, a CD3 zeta chain, CD28, CD3epsilon, CD45, CD4, CD5, CD9, CD16, CD22, CD33, CD37, CD64, CD80, CD86, CD134, CD137, ICOS, CD154, IG1, IG4, functional derivatives thereof, and combinations thereof;
(c) the transmembrane domain comprises a transmembrane domain of CD28, a T-cell receptor α or β chain, a CD3 zeta chain, a CD3− Epsilon, CD45, CD4, CD5, CD7, CD8, CD9, CD16, CD22, CD33, CD37, CD64, CD80, CD86, CD68, CD134, CD137, ICOS, CD41, CD154, and combinations thereof,
(d) the co-stimulatory signaling region comprises a CD28 signaling domain, a 4-1BB signaling domain, a CD3 Zeta signaling domain or a combination of one or more of a CD28 signaling domain, a 4-1BB signaling domain and a CD3 Zeta signaling domain;
(e) the signaling domain comprises CD28, IL-15 receptor alpha, IL-15 receptor alpha cytoplasmic domain, CD80, CD86, CTLA-4, B7-H1/PD-L1, ICOS, B7-H2, PD-1, B7-H3, PD-L2, B7-H4, PDCD6, BTLA, CD40 Ligand/TNFSF5, 4-1BB Ligand/TNFSF9, GITR/TNFRSF18; BAFF/BLyS/TNFSF13B, GITR Ligand/TNFSF18, BAFF R/TNFRSF13C, HVEM/TNFRSF14, CD27/TNFRSF7, LIGHT/TNFSF14, OX40/TNFRSF4, CD30/TNFRSF8, OX40 Ligand/TNFSF4, CD30 Ligand/TNFSF8, TACI/TNFRSF13B, 2B4/CD244/SLAMF4, CD84/SLAMF5, BLAME/SLAMF8, CD229/SLAMF3, CD2, CRACC/SLAMF7, CD2F-10/SLAMF9, NTB-A/SLAMF6, CD48/SLAMF2, SLAM/CD150, CD58/LFA-3, Ikaros, CD53, Integrin alpha 4/CD49d, CD82/Kai-1, Integrin alpha 4 beta 1, CD90/Thy1, Integrin alpha 4 beta 7/LPAM-1, CD96, LAG-3, CD160, LMIR1/CD300A, CRTAM, TCL1A, DAP12, TIM-1/KIM-1/HAVCR, Dectin-1/CLEC7A, TIM-4, DPPIV/CD26, TSLP, EphB6, TSLP R, HLA-DR, OX40; CD30, CD40, CD7, CD258, Natural killer Group 2 member C (NKG2C), Natural killer Group 2 member D (NKG2D), CDS, ICAM-1, LFA-1 (CD1a/CD18), ICOS, a ligand that binds to CD83, ICAM-1, LFA-1 (CD1 la/CD18) and/or 4-1BB (CD137) or a fusion protein comprising two or more of the foregoing proteins; and
(f) said bifunctional molecule is a molecule according to the formula: 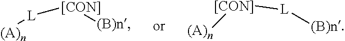
wherein:
n is an integer from 1 to 3;
n′ is an integer from 1 to 6;
(1) A is a moiety which is conjugated to the antigen binding domain of the chimeric antigen receptor (CAR) T cell by action of (i) a modified haloalkane dehydrogenase protein on a C₃-C₁₀ haloalkane when the antigen binding domain comprises a modified haloalkane dehydrogenase protein, (ii) a AGT protein reactive with a O6-benzylguanine moiety when the antigen binding domain comprises a AGT protein reactive with O6-benzylguanine derivatives or (iii) a AGT protein reactive with O2-benzylcytosine derivatives when the antigen binding domain is a AGT protein reactive with O2-benzylcytosine derivatives;
(2) B is a cancer binding moiety which binds to prostate specific membrane antigen (PSMA) on a cancer cell and which has the formula: 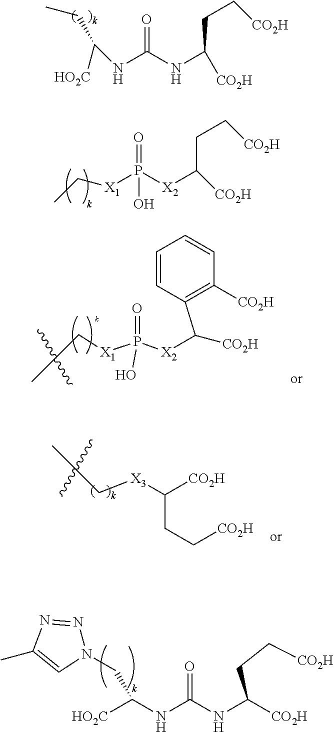
where X₁ and X₂ are each independently CH₂, O, NH or S;
X₃ is O, CH₂, NR¹, S(O), S(O)₂, —S(O)₂O, —OS(O)₂, or OS(O)₂O;
R¹ is H, a C₁-C₃ alkyl group, or a —C(O)(C₁-C₃) group;
k is an integer from 0 to 12; and
(3) L is a linker according to the chemical formula: 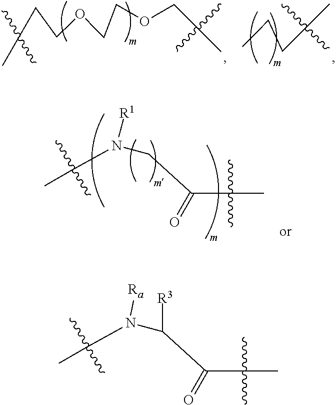
Where R¹ is H or a C₁-C₃ alkyl group;
Rₐ is H, C₁-C₃ alkyl or alkanol or forms a cyclic ring with R³ to form a proline or hydroxyproline unit or R³ is a side chain of an amino acid selected from the group consisting of methyl (from alanine), propyleneguanidine (from arginine), methylenecarboxyamide (from asparagine), ethanoic acid (from aspartic acid), thiol or di-thiol (from cysteine), ethylcarboxyamide (from glutamine), propanoic acid (from glutamic acid), hydrogen (from glycine), methyleneimidazole (from histidine), 1-methylpropane (from isoleucine), 2-methylpropane (from leucine), butyleneamine (from lysine), ethylmethylthioether (from methionine), phenylalanine (benzyl (from phenylalanine), pyrrolidine or hydroxypyrrolidine (from proline or hydroxyproline such that R³ forms a cyclic ring with Rₐ and the adjacent nitrogen group), methanol (from serine), ethanol (from threonine), methyleneindole (from tryptophan), tyrosine (methylene phenol) and valine (isopropyl);
m′ is an integer from 0 to 15;
each m is independently an integer from 1 to 100, or
L is a polyethylene glycol, polypropylene glycol or polypropylene-co-polyethylene glycol linker having between 1 and 100 glycol units, or
L is a linker according to the chemical formula: 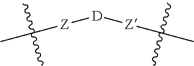
Where Z and Z′ are each independently a bond, —(CH₂)ᵢ—O, —(CH₂)ᵢ—S, —(CH₂)ᵢ—N—R, 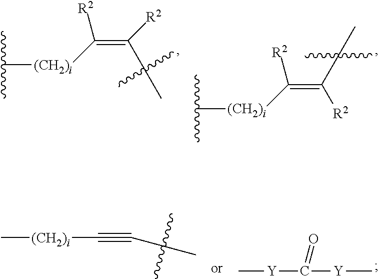
wherein said —(CH₂)ᵢ group, if present in Z or Z′, is bonded to a connector, CARBM moiety or cancer binding group PBM;
Each R is H, or a C₁-C₃ alkyl or alkanol group;
Each R² is independently H or a C₁-C₃ alkyl group;
Each Y is independently a bond, O, S or N—R;
Each i is independently an integer from 1 to 100;
D is 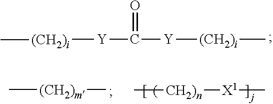
or
a bond, with the proviso that Z, Z′ and D are not each simultaneously bonds;
j is an integer from 1 to 100;
m′ is an integer from 1 to 100;
n is an integer from 1 to 100;
X¹ is O, S or N—R; and
R is H, or a C₁-C₃ alkyl or alkanol group; and
(4) CON is a bond or is a connector moiety selected from the group consisting of: 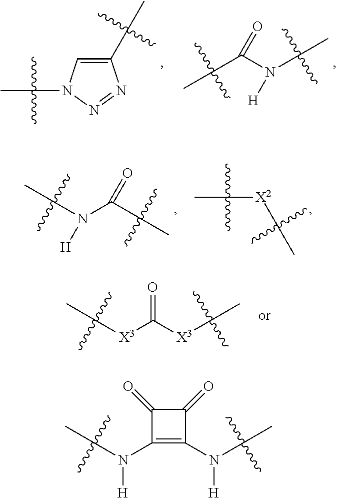
where X² is O, S, NR⁴, S(O), S(O)₂, —S(O)₂O, —OS(O)₂, or OS(O)₂O;
X³ is O, S, NR⁴; and
R⁴ is H, a C₁-C₃ alkyl or alkanol group, or a —C(O)(C₁-C₃) group, or
a pharmaceutically acceptable salt or stereoisomer thereof.

65. The CAR T cell according to claim 64 wherein the T cell is a helper (CD4⁺) T cell, a cytotoxic (CD8⁺) T cell, a central memory T cell (T_{CM} cell), an effector memory T cell, a regulatory T cell or a natural killer T cell (NKT cell).

66. The CAR T cell according to claim 64, wherein the T cell is obtained from a subject who suffers from prostate cancer.

67. The CAR T cell according to claim 64, wherein the signaling domain comprises two co-stimulatory domains combined with an activation domain in the cytoplasmic domain and/or the CAR T cell includes a signal sequence.

68. The CAR T cell according to claim 64 wherein
(a) the antigen binding domain of the chimeric antigen receptor CAR T cell is a modified haloalkane dehydrogenase protein and the bi-functional molecule which is conjugated to said CAR T cell is a molecule according to the formula: 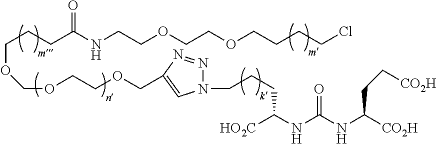
Wherein k′ is an integer from 0 to 6;
n′ is an integer from 0 to 20;
m′ is an integer from 0 to 5;
m′″ is an integer from 0 to 5, or
a pharmaceutically acceptable salt or stereoisomer thereof, or
(b) the antigen binding domain of the chimeric antigen receptor (CAR) T cell is a AGT protein reactive with O6-benzylguanine derivatives and the bi-functional molecule which is conjugated to said CAR T cell is a molecule according to the formula: 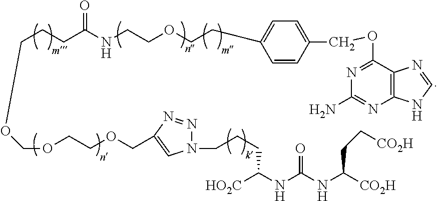
Where k′ is an integer from 0 to 6;
n′ is an integer from 0 to 20;
n″ is an integer from 0 to 16;
m′ is an integer from 0 to 5; and
m′″ is an integer from 0 to 5, or
a pharmaceutically acceptable salt or stereoisomer thereof, or
(c) the antigen binding domain of the chimeric antigen receptor (CAR) T cell is a AGT protein reactive with O2-cytosine derivatives and the bi-functional molecule which is conjugated to said CAR T cell is a molecule according to the formula: 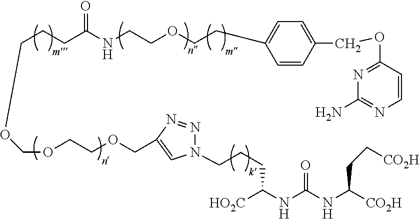
Where k′ is an integer from 0 to 6;
n′ is an integer from 0 to 20;
n″ is an integer from 0 to 16;
m′ is an integer from 0 to 5; and
m′″ is an integer from 0 to 5, or
a pharmaceutically acceptable salt or stereoisomer thereof, or

69. The CAR T cell according to claim 68 wherein the bifunctional molecule is a molecule according to the chemical formula: 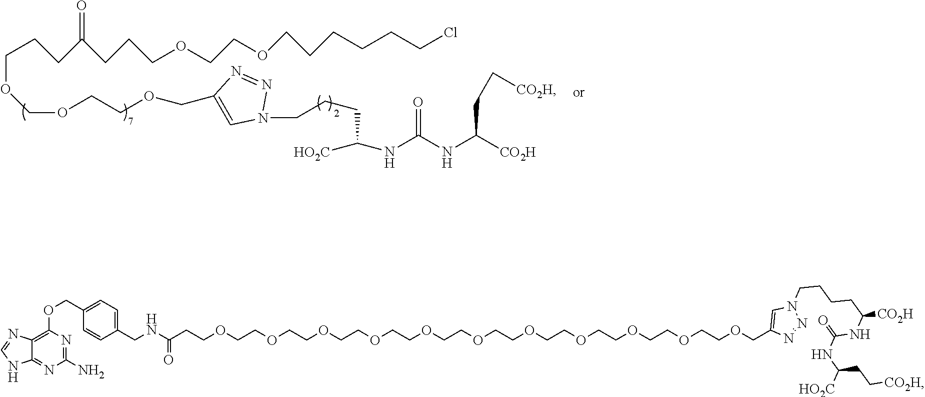
a pharmaceutically acceptable salt thereof.

70. The CAR T cell according to claim 69 wherein the bifunctional molecule is a molecule according to the chemical formula: 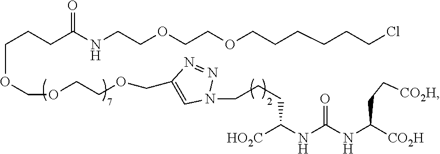
or
a pharmaceutically acceptable salt thereof.

71. The CAR T cell according to claim 69 wherein the bifunctional molecule is a molecule according to the chemical formula: 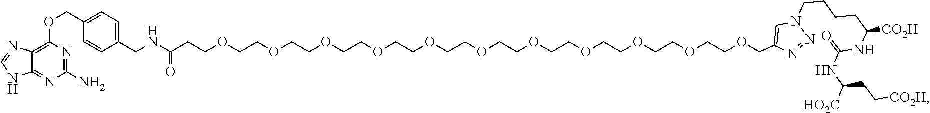 [SMILES: Nc1nc(OCc2ccc(CNC(=O)CCOCCOCCOCCOCCOCCOCCOCCOCCOCCOCCOCc3cn(CCCC[C@H](NC(=O)N[C@@H](CCC(=O)O)C(=O)O)C(=O)O)nn3)cc2)c2nc[nH]c2n1]
or
a pharmaceutically acceptable salt thereof.

72. A bi-functional molecule according to the chemical formula: 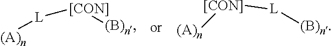
wherein:
n is an integer from 1 to 3;
n′ is an integer from 1 to 6;
A is (1) a C₃-C₁₀ haloalkane, (2) a O6-benzylguanine moiety or (3) a O2-benzylcytosine moiety;
B is a cancer binding moiety (PBM) which has the formula: 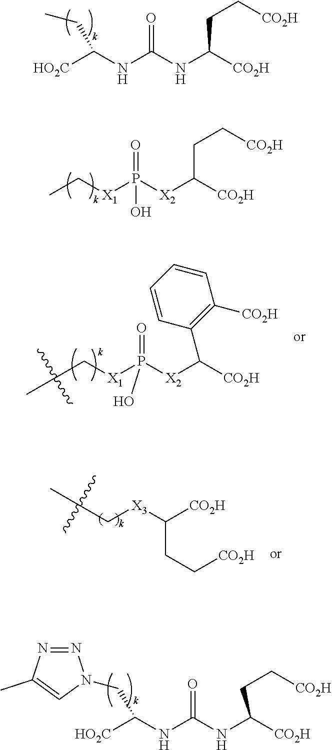
where X₁ and X₂ are each independently CH₂, O, NH or S;
X₃ is O, CH₂, NR¹, S(O), S(O)₂, —S(O)₂O, —OS(O)₂, or OS(O)₂O;
R¹ is H, a C₁-C₃ alkyl group, or a —C(O)(C₁-C₃) group;
k is an integer from 0 to 20; and
L is a linker according to the chemical formula: 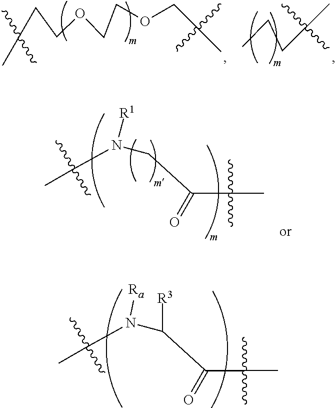
Where R¹ is H or a C₁-C₃ alkyl group;
Rₐ is H, C₁-C₃ alkyl or alkanol or forms a cyclic ring with R³ to form a proline or hydroxyproline unit and R³ is a side chain of an amino acid selected from the group consisting of methyl (from alanine), propyleneguanidine (from arginine), methylenecarboxyamide (from asparagine), ethanoic acid (from aspartic acid), thiol or di-thiol (from cysteine), ethylcarboxyamide (from glutamine), propanoic acid (from glutamic acid), hydrogen (from glycine), methyleneimidazole (from histidine), 1-methylpropane (from isoleucine), 2-methylpropane (from leucine), butyleneamine (from lysine), ethylmethylthioether (from methionine), phenylalanine (benzyl (from phenylalanine), pyrrolidine or hydroxypyrrolidine (from proline or hydroxyproline such that R³ forms a cyclic ring with Rₐ and the adjacent nitrogen group), methanol (from serine), ethanol (from threonine), methyleneindole (from tryptophan), tyrosine (methylene phenol) and valine (isopropyl);
m′ is an integer from 0 to 15; and
each m is independently an integer from 1 to 100, or
L is a polyethylene glycol, polypropylene glycol or polypropylene-co-polyethylene glycol linker having between 1 and 100 glycol units, or
L is a linker according to the chemical formula: 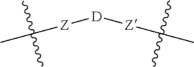
Where Z and Z′ are each independently a bond, —(CH₂)ᵢ—O, —(CH₂)ᵢ—S, —(CH₂)ᵢ—N—R, 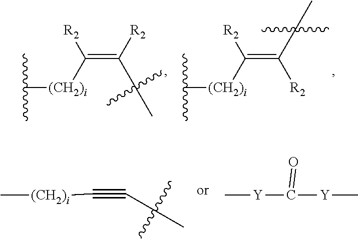
wherein said —(CH₂)ᵢ group, if present in Z or Z′, is bonded to a connector, CARBM moiety or cancer binding group PBM;
Each R is H, or a C₁-C₃ alkyl or alkanol group;
Each R² is independently H or a C₁-C₃ alkyl group;
Each Y is independently a bond, O, S or N—R;
Each i is independently an integer from 1 to 100;
D is 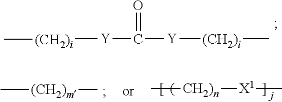
or
a bond, with the proviso that Z, Z′ and D are not each simultaneously bonds;
j is an integer from 1 to 100;
m′ is an integer from 1 to 100;
n is an integer from 1 to 75;
X¹ is O, S or N—R; and
R is H, or a C₁-C₃ alkyl or alkanol group; and
CON is a bond or is a connector moiety selected from the group consisting of: 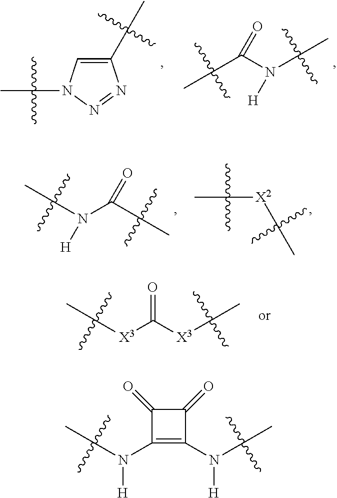
where X² is O, S, NR⁴, S(O), S(O)₂, —S(O)₂O, —OS(O)₂, or OS(O)₂O;
X³ is O, S, NR⁴; and
R⁴ is H, a C₁-C₃ alkyl or alkanol group, or a —C(O)(C₁-C₃) group, or
a pharmaceutically acceptable salt or stereoisomer thereof.

73. The bi-functional molecule according to claim 72 according to the chemical structure: 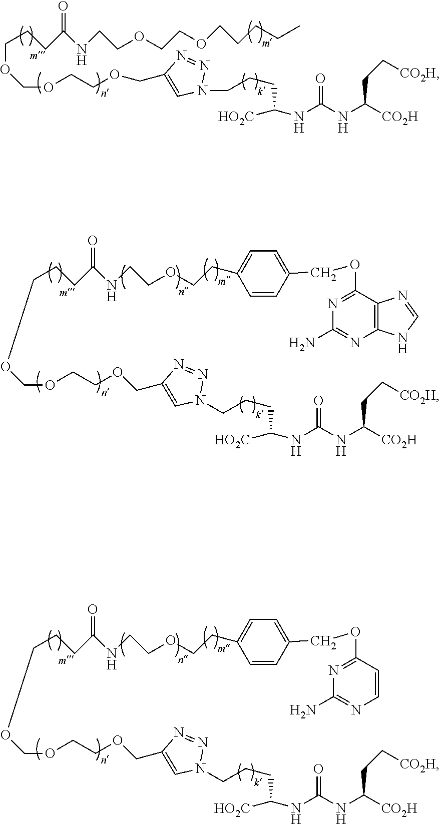
a pharmaceutically acceptable salt thereof.

74. A compound according to the chemical formula: 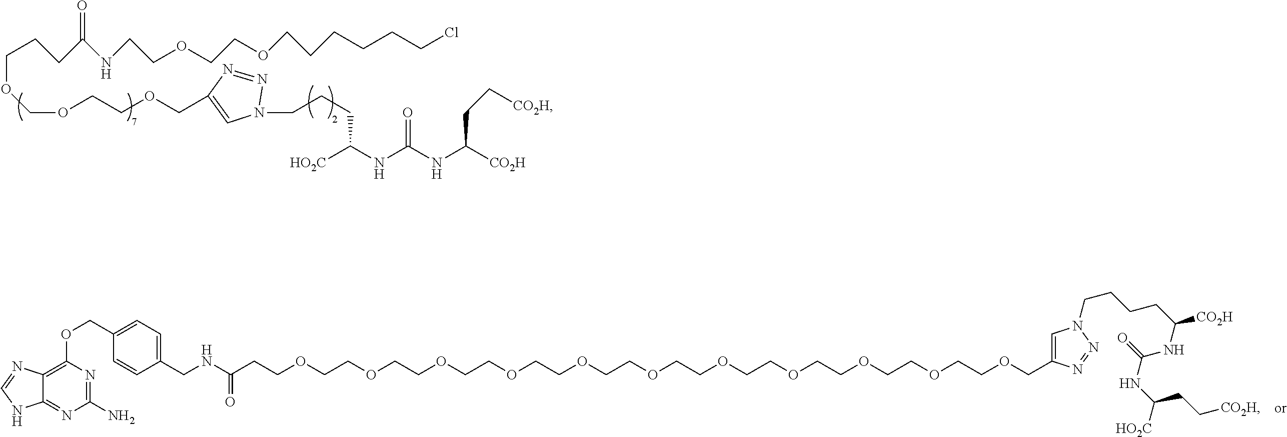 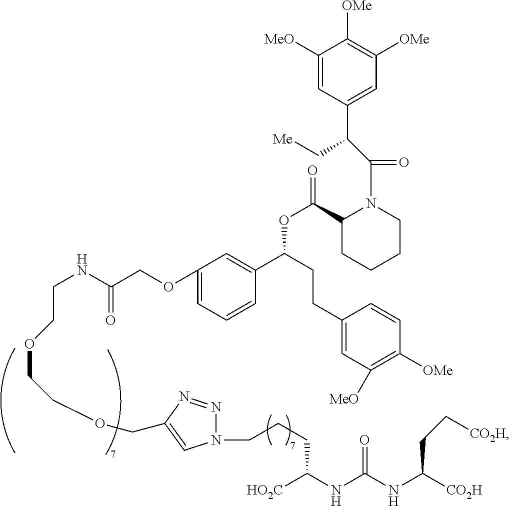
or
a pharmaceutically acceptable salt or stereoisomer thereof.

75. A nucleic acid molecule encoding a chimeric antigen receptor comprising:
(a) an antigen binding domain comprising a modified haloalkane dehydrogenase protein reactive with chloroalkane derivatives, a O6-methylguanine-methyltransferase (AGT) protein reactive with O6-benzylguanine derivatives or a AGT protein reactive with O2-benzylcytosine derivatives;
(b) a hinge domain comprising a hinge domain of CD28, 4-1BB, OX40, CD3-zeta, CD 8, CD-8 alpha, T cell receptor α or R chain, a CD3 zeta chain, CD3epsilon, CD45, CD4, CD5, CD9, CD16, CD22, CD33, CD37, CD64, CD80, CD86, CD134, CD137, ICOS, CD154, IG1, IG4, functional derivatives thereof, and combinations thereof;
(c) a transmembrane domain comprising a transmembrane domain of CD28, a T-cell receptor α or R chain, a CD3 zeta chain, a CD3−Epsilon, CD45, CD4, CD5, CD7, CD8, CD9, CD16, CD22, CD33, CD37, CD64, CD80, CD86, CD68, CD134, CD137, ICOS, CD41, CD154, and combinations thereof,
(d) a co-stimulatory signaling region comprising a C28 signaling domain, a 4-1BB signaling domain, a CD3 Zeta signaling domain or a combination of one or more of a C28 signaling domain, a 4-1BB signaling domain and a CD3 Zeta signaling domain; and
(e) a signaling domain comprising CD28, IL-15 receptor alpha, IL-15 receptor alpha cytoplasmic domain, CD80, CD86, CTLA-4, B7-H1/PD-L1, ICOS, B7-H2, PD-1, B7-H3, PD-L2, B7-H4, PDCD6, BTLA, CD40 Ligand/TNFSF5, 4-1BB Ligand/TNFSF9, GITR/TNFRSF18; BAFF/BLyS/TNFSF13B, GITR Ligand/TNFSF18, BAFF R/TNFRSF13C, HVEM/TNFRSF14, CD27/TNFRSF7, LIGHT/TNFSF14, CD27 Ligand/TNFSF7, OX40/TNFRSF4, CD30/TNFRSF8, OX40 Ligand/TNFSF4, CD30 Ligand/TNFSF8, TACI/TNFRSF13B, 2B4/CD244/SLAMF4, CD84/SLAMF5, BLAME/SLAMF8, CD229/SLAMF3, CD2, CRACC/SLAMF7, CD2F-10/SLAMF9, NTB-A/SLAMF6, CD48/SLAMF2, SLAM/CD150, CD58/LFA-3, Ikaros, CD53, Integrin alpha 4/CD49d, CD82/Kai-1, Integrin alpha 4 beta 1, CD90/Thy1, Integrin alpha 4 beta 7/LPAM-1, CD96, LAG-3, CD160, LMIR1/CD300A, CRTAM, TCL1A, DAP12, TIM-1/KIM-1/HAVCR, Dectin-1/CLEC7A, TIM-4, DPPIV/CD26, TSLP, EphB6, TSLP R, HLA-DR, OX40; CD30, CD40, CD7, CD258, Natural killer Group 2 member C (NKG2C), Natural killer Group 2 member D (NKG2D), CDS, ICAM-1, LFA-1 (CD1a/CD18), B7-H3, PD-1, ICOS, a ligand that binds to CD83, ICAM-1, LFA-1 (CD11a/CD18), ICOS, and/or 4-1BB (CD137), a variant of one of the foregoing proteins or a fusion protein comprising two or more of the foregoing proteins.

76. A vector comprising a nucleic acid molecule according to claim 75.

77. An isolated host cell which is transduced with a vector according to claim 76.

78. The isolated host cell according to claim 77 wherein the host cell is a T cell.

79. The isolated host cell according to claim 80 wherein said T cell is a helper (CD4⁺) T cell, a cytotoxic (CD8⁺) T cell, a central memory T cell (T_{CM} cell), an effector memory T cell, a regulatory T cell or a natural killer T cell (NKT cell).

80. The chimeric antigen receptor (CAR) T cell of claim 64, wherein the chimeric antigen receptor (CAR) T cell comprises a reporter.

81. A chimeric antigen receptor (CAR) comprising an antigen binding domain, a hinge domain, a transmembrane domain, a co-stimulatory signaling region and a signaling domain, wherein
a) the antigen binding domain comprises a modified haloalkane dehydrogenase protein reactive with chloroalkane derivatives, a O6-methylguanine-methyltransferase (AGT) protein reactive with O6-benzylguanine derivatives or a AGT protein reactive with O2-benzylcytosine derivatives;
(b) the hinge domain comprises a hinge domain of CD28, 4-1BB, OX40, CD3-zeta, CD 8, CD-8 alpha, T cell receptor α or R chain, a CD3 zeta chain, CD28, CD3epsilon, CD45, CD4, CD5, CD9, CD16, CD22, CD33, CD37, CD64, CD80, CD86, CD134, CD137, ICOS, CD154, IG1, IG4, and combinations thereof,
(c) the transmembrane domain comprises a transmembrane domain of CD28, a T-cell receptor α or β chain, a CD3 zeta chain, a CD3−Epsilon, CD45, CD4, CD5, CD7, CD8, CD9, CD16, CD22, CD33, CD37, CD64, CD80, CD86, CD68, CD134, CD137, ICOS, CD41, CD154, and combinations thereof,
(d) the co-stimulatory signaling region comprises a C28 signaling domain, a 4-1BB signaling domain, a CD3 Zeta signaling domain or a combination of one or more of a C28 signaling domain, a 4-1BB signaling domain and a CD3 Zeta signaling domain; and
(e) the signaling domain comprises CD28, IL-15 receptor alpha, IL-15 receptor alpha cytoplasmic domain, CD80, CD86, CTLA-4, B7-H1/PD-L1, ICOS, B7-H2, PD-1, PD-L2, B7-H4, PDCD6, BTLA, CD40 Ligand/TNFSF5, 4-1BB Ligand/TNFSF9, GITR/TNFRSF18; BAFF/BLyS/TNFSF13B, GITR Ligand/TNFSF18, BAFF R/TNFRSF13C, HVEM/TNFRSF14, CD27/TNFRSF7, LIGHT/TNFSF14, CD27 Ligand/TNFSF7, OX40/TNFRSF4, CD30/TNFRSF8, OX40 Ligand/TNFSF4, CD30 Ligand/TNFSF8, TACI/TNFRSF13B, 2B4/CD244/SLAMF4, CD84/SLAMF5, BLAME/SLAMF8, CD229/SLAMF3, CD2, CRACC/SLAMF7, CD2F-10/SLAMF9, NTB-A/SLAMF6, CD48/SLAMF2, SLAM/CD150, CD58/LFA-3, Ikaros, CD53, Integrin alpha 4/CD49d, CD82/Kai-1, Integrin alpha 4 beta 1, CD90/Thy1, Integrin alpha 4 beta 7/LPAM-1, CD96, LAG-3, CD160, LMIR1/CD300A, CRTAM, TCL1A, DAP12, TIM-1/KIM-1/HAVCR, Dectin-1/CLEC7A, TIM-4, DPPIV/CD26, TSLP, EphB6, TSLP R, HLA-DR, OX40; CD30, CD40, CD7, CD258, Natural killer Group 2 member C (NKG2C), Natural killer Group 2 member D (NKG2D), CDS, ICAM-1, LFA-1 (CD1a/CD18), B7-H3, ICOS, a ligand that binds to CD83, ICAM-1, LFA-1 (CD11a/CD18), and/or 4-1BB (CD137) or a fusion protein comprising two or more of the foregoing proteins.

82. A composition comprising a population of chimeric antigen receptor (CAR) T cells according to claim 64.

83. A method of diagnosing prostate cancer in a subject, the method comprising contacting a sample of prostate cells obtained from the subject with chimeric antigen receptor (CAR) T cells according to claim 82, measuring levels of reporter in the sample prostate cells and diagnosing the presence or absence of prostate cancer based on measured detectable reporter levels in comparison to a standard for a patient without prostate cancer and/or a standard for a patient with prostate cancer.

84. A method of treating a subject who suffers from prostate cancer, the method comprising administering to the subject a therapeutically effective amount of a composition comprising a population of CAR T cells according to claim 64.

85. The method of treatment according to claim 84, wherein the prostate cancer is metastatic or recurrent prostate cancer.

86. The method of claim 84, wherein the chimeric antigen receptor (CAR) T cells are derived from T cells obtained from the subject.

87. The method of treatment according to claim 84 comprising performing the steps of T cell apheresis, retroviral or lentiviral CAR transduction of said T cells, T cell expansion, and host conditioning before administration of a population of chimeric antigen receptor (CAR) T cells to the subject.

88. A method of treating a subject who suffers from a cancer which overexpresses prostate specific membrane antigen (PSMA) selected from the group consisting of stomach, colon, rectal, liver, pancreatic, lung, breast, cervix uteri, corpus uteri, ovary, testis, bladder, renal, brain/CNS, head and neck and throat cancer, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, the method comprising administering to the subject a therapeutically effective amount of a composition comprising a population of chimeric antigen receptor (CAR) T cells according to claim 66.
